(19) United States
(12) Patent Application Publication
(10) Pub. No.: US 2015/0150158 A1
(43) Pub. Date: May 28, 2015

(54) PLANTS HAVING ENHANCED YIELD-RELATED TRAITS AND METHOD FOR MAKING THE SAME

(71) Applicants: BASF Plant Science Company GmbH, Ludwigshafen (DE); Crop Functional Genomic Center, Seoul (KR)

(72) Inventors: Christophe Reuzeau, La Chapelle Gonaguet (FR); Yang Do Choi, Seoul (KR); Ju Kon Kim, Sungnam (KR); Jin Seo Jeong, Seoul (KR)

(73) Assignees: BASF Plant Science Company GmbH, Ludwigshafen (DE); Crop Functional Genomic Center, Seoul (KR)

(21) Appl. No.: 14/353,162

(22) PCT Filed: Oct. 19, 2012
(86) PCT No.: PCT/IB2012/055733

§ 371 Date: Apr. 21, 2014

Related U.S. Application Data
(60) Provisional application No. 61/549,803, filed on Oct. 21, 2011.

(30) Foreign Application Priority Data
Oct. 21, 2011 (EP) 11186254.6

(51) Int. Cl. C12N 15/82

(52) U.S. Cl. CPC C12N 15/8261
USPC 800/287; 800/290; 800/298; 435/419; 435/320.1; 800/320.1; 800/320.3; 800/320; 435/412

(57) ABSTRACT

A method for enhancing yield-related traits in plants by modulating expression in a plant of a nucleic acid up-regulated upon overexpression of a NAC1 or NAC5-encoding gene, referred to herein as a NUG or NAC up-regulated gene, is provided. Plants having modulated expression of a NUG, which plants have enhanced yield-related traits relative to corresponding wild type plants or other control plants, are also provided. A method for conferring abiotic stress tolerance in plants, comprising modulating expression of a nucleic acid encoding a NAC1 or NAC5 polypeptide in plants grown under abiotic stress conditions, is also provided. Plants expressing a nucleic acid encoding a NAC1 or NAC5 polypeptide, aside from having increased abiotic stress tolerance, have enhanced yield-related traits and/or modified root architecture compared to corresponding wild type plants. Constructs useful in the methods and plants produced by the methods are also provided.

### BACKGROUND

The present invention relates generally to the field of molecular biology and concerns a method for enhancing yield-related traits in plants by modulating expression in a plant of a nucleic acid up-regulated upon overexpression of a NAC1 or NAC5-encoding gene, referred to herein as a NUG or “NAC up-regulated gene”. The present invention also concerns plants having modulated expression of a NUG, which plants have enhanced yield-related traits relative to corresponding wild type plants or other control plants. The present invention also relates to a method for conferring abiotic stress tolerance in plants, comprising modulating expression of a nucleic acid encoding a NAC1 or NAC5 polypeptide in plants grown under abiotic stress conditions. Plants expressing a nucleic acid encoding a NAC1 or NAC5 polypeptide, aside from having increased abiotic stress tolerance, have enhanced yield-related traits and/or modified root architecture compared to corresponding wild type plants. The invention also provides constructs useful in the methods of the invention and plants produced by the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds.

They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta 218, 1-14, 2003). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors. Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

Among the widely studied drought-responsive genes are the transcriptional regulators belonging to NAC (NAM, ATAF, and CUC) gene-family. Members of the NAC gene-family are found only in plants and many are involved in stress responses. NAC proteins consist of a highly conserved N-terminal end, the DNA binding domain that can form a β-sheet structure where proteins form into either a homodimer or a heterodimer (Ernst et al., 2004; Hegedus et al., 2003; Jeong et al. 2009; Takasaki et al., 2010; Xie et al., 2000), and a highly variable C-terminal region (Zheng et al 2009).

WO 2007/144190 describes the use of various NAC-encoding nucleotide sequences for increasing yield in plants under non-stress conditions or under mild drought conditions.

It has now been found that various yield-related traits may be enhanced in plants by modulating expression in a plant of a nucleic acid up-regulated upon overexpression of a NAC1 or NAC5 gene/nucleic acid. Nucleic acids up-regulated upon overexpression of a NAC1 or NAC5 gene/nucleic acid are referred to herein as NUGs or NAC up-regulated genes.

It has also been found that overexpressing a nucleic acid encoding a NAC1 or NAC5 polypeptide in plants grown under abiotic stress conditions gives plants having enhanced yield-related traits and/or modified root architecture compared to corresponding wild type plants, wherein said nucleic acid is operably linked to a tissue-specific promoter.

It has also now been found that abiotic stress tolerance may be conferred in plants by overexpressing a nucleic acid encoding a NAC1 or NAC5 polypeptide in a plant, which nucleic acid is operably linked to a tissue-specific promoter.

### DETAILED DESCRIPTION OF THE INVENTION

The present invention shows that modulating expression in a plant of nucleic acid up-regulated upon overexpression of a NAC1 or NAC5 gene/nucleic acid, referred to herein as a NUG or NAC up-regulated gene, gives plants having enhanced yield-related traits relative to control plants.

The present invention also shows that overexpressing a nucleic acid encoding a NAC1 or NAC5 polypeptide in plants grown under abiotic stress conditions gives plants having enhanced yield-related traits and/or modified root architecture relative to corresponding wild type plants, wherein said nucleic acid is operably linked to a tissue-specific promoter.

### 1. NUG or NAC Up-Regulated Genes

According to a first aspect of the present invention, there is provided a method for enhancing yield-related traits in plants relative to control plants, comprising modulating expression in a plant of a NUG and optionally selecting for plants having enhanced yield-related traits.

According to further aspect of the present invention, there is provided a method for producing plants having enhanced yield-related traits relative to control plants, comprising the steps of modulating expression in a plant of a nucleic acid encoding a NUG polypeptide as described herein and optionally selecting for plants having enhanced yield-related traits.

A preferred method for modulating, preferably increasing, expression of a nucleic acid encoding a NUG polypeptide is by introducing and expressing in a plant a nucleic acid encoding a NUG polypeptide.

Any reference hereinafter to a “protein useful in the methods of the invention” is taken to mean a NUG polypeptide as defined herein. Any reference hereinafter to a “nucleic acid useful in the methods of the invention” is taken to mean a nucleic acid capable of encoding a NUG polypeptide. The nucleic acid to be introduced into a plant (and therefore useful in performing the methods of the invention) is any nucleic acid encoding the type of protein which will now be described, hereinafter also named “NUG nucleic acid” or “NUG gene”.

A “NUG polypeptide” as defined herein refers to any of the polypeptides described in Table A or Table B or a homologue of any of the polypeptides described in Table A or Table B.

An “NUG” or “NUG nucleic acid” as defined herein refers to any gene/nucleic acid capable of encoding a NUG polypeptide or homologue thereof as defined herein.

Examples of nucleic acids encoding NUG polypeptides are given in Table A and Table B herein; such nucleic acids are useful in performing the methods of the invention. Homologues of NUG polypeptides are also useful in performing the methods of the invention.

Table A shows up-regulated root-expressed genes in RCc3:OsNAC1 and GOS2:OsNAC1 plants in comparison to non-transgenic controls.

Table B shows up-regulated genes in RCc3:OsNAC5 and/or GOS2:OsNAC5 plants in comparison to non-transgenic controls.

Particularly preferred NUGs for use in the methods of the invention include the following:
a) O-methyltransferases, particularly Os09g0344500, OS10g0118000, OS10g0118200.
b) AAA-type ATPase, particularly OS09g0445700.
c) Leucine rich repeate, particularly OS08g0202300.
d) DNA binding/homeodomain, particularly OS11g0282700.
e) Oxidoreductase, 20G-Fe(II)oxygenase, OS04g0581100.
f) Calcium transporting ATPase, particularly OS10g0418100.
g) 9-cis epoxycaretenoid dioxygenase, particularly OS07g0154100.
h) cinnamoyl CoA Reductase 1, particularly OS02g0811800.
i) LLR kinase, particularly OS07g0251800.
j) WRKY40, particularly OS09g0417600.
k) Germin-like GLP oxidoreductase, particularly OS03g0694000.
l) C4 dicarboxylate transporter, particularly OS04g0574700.
m) Fructose bisphosphase aldolase, particularly OS08g0120600.
n) MnT, particularly OS10g0118200.
o) Oxo phytodienoic acid reductase, particularly OS06g0215900.
p) Cytochrome p450, particularly OS12g0150200.

The NUG polypeptide or homologue thereof is defined herein as having at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%_{,} 97%, 98%, 99% or 100% overall sequence identity to one or more of the polypeptide sequences given in Table A or Table B.

Also included within the term “homologue” are orthologues and paralogues of the NUG polypeptides given in Tables A and B, the terms “orthologues” and “paralogues” being as defined herein. Orthologues and paralogues may readily be identified by performing a so-called reciprocal blast search as described in the definitions section.

The overall sequence identity may be determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). In one embodiment the sequence identity level is determined by comparison of the polypeptide sequences over the entire length of the polypeptide sequences in Table A and Table B.

The sequence identity level may also be determined by comparison of one or more conserved domains or motifs present in one of the polypeptide sequences in Table A or Table B compared to corresponding conserved domains or motifs in homologous family members of the NUG in question. Compared to overall sequence identity, the sequence identity will generally be higher when only conserved domains or motifs are considered. The terms “domain”, “signature” and “motif” are defined in the “definitions” section herein.

Tools for identifying domains are known in the art and comprise querying databases like InterPro (Hunter et al., Nucleic Acids Res. 37 (Database Issue):D224-228, 2009) with a protein sequence from Table A or B, or of homologous sequences therefrom. Also the identification of motifs is known in the art, for example by using the MEME algorithm (Bailey and Elkan, Proceedings of the Second International Conference on Intelligent Systems for Molecular Biology, pp. 28-36, AAAI Press, Menlo Park, Calif., 1994). To this end, a set of homologous protein sequences is used as input. At each position within a MEME motif, the residues are shown that are present in the query set of sequences with a frequency higher than 0.2. Residues within square brackets represent alternatives.

The nucleic acid sequences encoding NUG polypeptides confer information for synthesis of the NUG that increases yield or yield related traits as described herein, when such a nucleic acid sequence of the invention is transcribed and translated in a living plant cell.

Nucleic acid variants may also be useful in practising the methods of the invention. Examples of such variants include nucleic acids encoding homologues and derivatives of any one of the amino acid sequences given in Table A or Table B herein, the terms “homologue” and “derivative” being as defined herein.

Also useful in the methods of the invention are nucleic acids encoding homologues and derivatives of orthologues or paralogues of any one of the amino acid sequences given in Table A or Table B herein. Homologues and derivatives useful in the methods of the present invention have substantially the same biological and functional activity as the unmodified protein from which they are derived. Further variants useful in practising the methods of the invention are variants in which codon usage is optimised or in which miRNA target sites are removed.

Further nucleic acid variants useful in practising the methods of the invention include portions of nucleic acids encoding NUG polypeptides, nucleic acids hybridising to nucleic acids encoding NUG polypeptides, splice variants of nucleic acids encoding NUG polypeptides, allelic variants of nucleic acids encoding NUG polypeptides and variants of nucleic acids encoding NUG polypeptides obtained by gene shuffling. The terms hybridising sequence, splice variant, allelic variant and gene shuffling are as described herein.

Nucleic acids encoding NUG polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A or Table B herein, or a portion of a nucleic acid encoding an orthologue, paralogue or homologue of any of the amino acid sequences given in Table A or Table B herein.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a NUG polypeptide as defined herein or at least part thereof, and have substantially the same biological activity as the amino acid sequences given in Table A or Table B herein. Preferably, the portion is a portion of any one of the nucleic acids given in Table A or Table B herein, or is a portion of a nucleic acid encoding an orthologue or paralogue of any one of the amino acid sequences given in Table A or Table B. Preferably the portion is at least 500, 550, 600, 650, 700, 750, 800, 850, 900, 950, 1000 consecutive nucleotides in length, the consecutive nucleotides being of any one of the nucleic acid sequences given in Table A or Table B, or of a nucleic acid encoding an orthologue or paralogue of any one of the amino acid sequences given in Table A or Table B herein.

Another nucleic acid variant useful in the methods of the invention is a nucleic acid capable of hybridising, under reduced stringency conditions, preferably under stringent conditions, with a nucleic acid encoding a NUG polypeptide as defined herein, or with a portion as defined herein. According to the present invention, there is provided a method for enhancing yield-related traits in plants, comprising introducing and expressing in a plant a nucleic acid capable of hybridizing to a nucleic acid encoding any one of the proteins given in Table A or Table B, or to a nucleic acid encoding an orthologue, paralogue or homologue of any of the proteins given in Table A or Table B.

Hybridising sequences useful in the methods of the invention encode a NUG polypeptide as defined herein having substantially the same biological activity as the amino acid sequence given in Table A or Table B encoded by the nucleic acid to which the hybridising sequence hybridises. Preferably, the hybridising sequence is capable of hybridising to the complement of a nucleic acid encoding any one of the proteins given in Table A or Table B, or to a portion of any of these sequences, a portion being as defined herein, or the hybridising sequence is capable of hybridising to the complement of a nucleic acid encoding an orthologue or paralogue of any one of the amino acid sequences given in Table A or Table B. The hybridization conditions may be medium stringency conditions or high stringency conditions, as defined herein.

Preferably, the hybridising sequence encodes a polypeptide with an amino acid sequence which comprises at least some of the motifs or conserved regions present in the polypeptide sequence encoded by the nucleic acid to which the hybridising sequence hybridises and/or has the same biological activity as the polypeptide encoded by the nucleic acid to which the hybridising sequence hybridises and/or has at least 50%, 55%, 60%, 65%, 70%, 75%, 80%, 85%, 90% or 95% or more sequence identity to the polypeptide encoded by the nucleic acid to which the hybridising sequence hybridises.

In another embodiment, there is provided a method for enhancing yield-related traits in plants, comprising introducing and expressing in a plant a splice variant or an allelic variant of a nucleic acid encoding any one of the proteins given in Table A or Table B herein, or a splice variant or an allelic variant of a nucleic acid encoding an orthologue, paralogue or homologue of any of the amino acid sequences given in Table A or Table B.

Preferred splice variants or allelic variants are those where the amino acid sequence encoded by the splice variant or allelic variant comprises at least some of the motifs or other conserved regions found in the non-variant sequence and/or has the same biological activity as the non-variant sequence and/or has at least 50%, 55%, 60%, 65%, 70%, 75%, 80%, 85%, 90% or 95% or more sequence identity to the non-variant sequence. Allelic variants exist in nature, and encompassed within the methods of the present invention is the use of these natural alleles.

According to a further embodiment of the present invention, there is provided a method for enhancing yield-related traits in plants, comprising introducing and expressing in a plant a variant of any one of the nucleic acid sequences given in Table A or Table B, or comprising introducing and expressing in a plant a variant of a nucleic acid encoding an orthologue, paralogue or homologue of any of the amino acid sequences given in Table A or Table B, which variant nucleic acid is obtained by gene shuffling.

Preferably, the amino acid sequence encoded by the variant nucleic acid obtained by gene shuffling comprises at least some motifs or other conserved regions found in the non-variant sequence and/or has the same biological activity as the non-variant sequence and/or has at least 50%, 55%, 60%, 65%, 70%, 75%, 80%, 85%, 90% or 95% or more sequence identity to the non-variant sequence from which the variant is derived.

Furthermore, nucleic acid variants may also be obtained by site-directed mutagenesis. Several methods are available to achieve site-directed mutagenesis, the most common being PCR based methods (Current Protocols in Molecular Biology. Wiley Eds). NUG polypeptides differing from the sequences of Table A or Table B by one or several amino acids (substitution(s), insertion(s) and/or deletion(s) as defined herein) may equally be useful to increase the yield of plants in the methods and constructs and plants of the invention.

Nucleic acids encoding NUG polypeptides may be derived from any natural or artificial source. The nucleic acid may be modified from its native form in composition and/or genomic environment through deliberate human manipulation.

Preferably the NUG polypeptide-encoding nucleic acid is from a plant, further preferably from a monocotyledonous plant, more preferably from the family Poaceae, most preferably the nucleic acid is from Oryza sativa.

The present invention also extends to the use of recombinant chromosomal DNA comprising a nucleic acid sequence useful in the methods of the invention, wherein said nucleic acid is present in the chromosomal DNA as a result of recombinant methods, but is not in its natural genetic environment. In a further embodiment the recombinant chromosomal DNA of the invention is comprised in a plant cell.

Performance of the methods of the invention gives plants having enhanced yield-related traits. In a particular embodiment of the invention, performance of the methods of the invention gives plants having increased early vigour and/or increased yield and/or increased biomass and/or increased seed yield relative to control plants. The terms “early vigour”, “biomass”, “yield” and “seed yield” are described in more detail in the “definitions” section herein.

The present invention therefore provides a method for enhancing yield-related traits relative to control plants, comprising modulating expression in a plant of a nucleic acid encoding a NUG polypeptide as defined herein.

According to a further embodiment of the present invention, performance of the methods of the invention gives plants having increased growth rate relative to control plants. Therefore, according to the present invention, there is provided a method for increasing the growth rate of plants, which method comprises modulating expression in a plant of a nucleic acid encoding a NUG polypeptide as defined herein.

Performance of the methods of the invention gives plants grown under non-stress conditions or under mild drought conditions enhanced yield-related traits relative to control plants grown under comparable conditions. Therefore, according to the present invention, there is provided a method for enhancing yield-related traits in plants grown under non-stress conditions or under mild drought conditions, which method comprises modulating expression in a plant of a nucleic acid encoding a NUG polypeptide.

Performance of the methods of the invention gives plants grown under conditions of drought, enhanced yield-related traits relative to control plants grown under comparable conditions. Therefore, according to the present invention, there is provided a method for enhancing yield-related traits in plants grown under conditions of drought which method comprises modulating expression in a plant of a nucleic acid encoding a NUG polypeptide.

Performance of the methods of the invention gives plants grown under conditions of nutrient deficiency, particularly under conditions of nitrogen deficiency, enhanced yield-related traits relative to control plants grown under comparable conditions. Therefore, according to the present invention, there is provided a method for enhancing yield-related traits in plants grown under conditions of nutrient deficiency, which method comprises modulating expression in a plant of a nucleic acid encoding a NUG polypeptide.

Performance of the methods of the invention gives plants grown under conditions of salt stress, enhanced yield-related traits relative to control plants grown under comparable conditions. Therefore, according to the present invention, there is provided a method for enhancing yield-related traits in plants grown under conditions of salt stress, which method comprises modulating expression in a plant of a nucleic acid encoding a NUG polypeptide.

The invention also provides genetic constructs and vectors to facilitate introduction and/or expression in plants of nucleic acids encoding NUG polypeptides. The gene constructs may be inserted into vectors, which may be commercially available, suitable for transforming into plants or host cells and suitable for expression of the gene of interest in the transformed cells. The invention also provides use of a gene construct as defined herein in the methods of the invention.

More specifically, the present invention provides a construct comprising:
(a) a nucleic acid encoding a NUG polypeptide as defined above;
(b) one or more control sequences capable of driving expression of the nucleic acid sequence of (a); and optionally
(c) a transcription termination sequence.

Preferably, the nucleic acid encoding a NUG polypeptide is as defined above. The term “control sequence” and “termination sequence” are as defined herein.

The genetic construct of the invention may be comprised in a host cell, plant cell, seed, agricultural product or plant. Plants or host cells are transformed with a genetic construct such as a vector or an expression cassette comprising any of the nucleic acids described above. Thus the invention further provides plants or host cells transformed with a construct as described above. In particular, the invention provides plants transformed with a construct as described above, which plants have increased yield-related traits as described herein.

In one embodiment the genetic construct of the invention confers increased yield or yield related traits(s) to a plant when it has been introduced into said plant, which plant expresses the nucleic acid encoding the NUG comprised in the genetic construct. In another embodiment the genetic construct of the invention confers increased yield or yield related traits(s) to a plant comprising plant cells in which the construct has been introduced, which plant cells express the nucleic acid encoding the NUG comprised in the genetic construct.

The skilled artisan is well aware of the genetic elements that must be present on the genetic construct in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence, but preferably the promoter is of plant origin. A constitutive promoter is particularly useful in the methods. See the “Definitions” section herein for definitions of the various promoter types.

The constitutive promoter is preferably a ubiquitous constitutive promoter of medium strength. More preferably it is a plant derived promoter, e.g. a promoter of plant chromosomal origin, such as a GOS2 promoter or a promoter of substantially the same strength and having substantially the same expression pattern (a functionally equivalent promoter), more preferably the promoter is the promoter GOS2 promoter from rice. See the “Definitions” section herein for further examples of constitutive promoters.

Optionally, one or more terminator sequences may be used in the construct introduced into a plant. Those skilled in the art will be aware of terminator sequences that may be suitable for use in performing the invention. Preferably, the construct comprises an expression cassette comprising a constitutive promoter (such as GOS2), operably linked to the nucleic acid encoding the NUG polypeptide. The construct may further comprises a terminator (such as a zein terminator) linked to the 3′ end of the NUG coding sequence. Furthermore, one or more sequences encoding selectable markers may be present on the construct introduced into a plant.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a nucleic acid encoding a NUG polypeptide is by introducing and expressing in a plant a nucleic acid encoding a NUG polypeptide; however the effects of performing the method, i.e. enhancing yield-related traits may also be achieved using other well-known techniques, including but not limited to T-DNA activation tagging, TILLING, homologous recombination. A description of these techniques is provided in the definitions section.

The invention also provides a method for the production of transgenic plants having enhanced yield-related traits relative to control plants, comprising introduction and expression in a plant of any nucleic acid encoding a NUG polypeptide as defined herein.

More specifically, the present invention provides a method for the production of transgenic plants having enhanced yield-related traits, comprising:
(i) introducing and expressing in a plant or plant cell a NUG polypeptide-encoding nucleic acid or a genetic construct comprising a NUG polypeptide-encoding nucleic acid; and
(ii) cultivating the plant cell under conditions promoting plant growth and development.

The nucleic acid of (i) may be any of the nucleic acids capable of encoding a NUG polypeptide as defined herein.

Cultivating the plant cell under conditions promoting plant growth and development, may or may not include regeneration and/or growth to maturity. Accordingly, in a particular embodiment of the invention, the plant cell transformed by the method according to the invention is regenerable into a transformed plant. In another particular embodiment, the plant cell transformed by the method according to the invention is not regenerable into a transformed plant, i.e. cells that are not capable to regenerate into a plant using cell culture techniques known in the art. While plants cells generally have the characteristic of totipotency, some plant cells cannot be used to regenerate or propagate intact plants from said cells. In one embodiment of the invention the plant cells of the invention are such cells. In another embodiment the plant cells of the invention are plant cells that do not sustain themselves in an autotrophic way.

The nucleic acid may be introduced directly into a plant cell or into the plant itself (including introduction into a tissue, organ or any other part of a plant). According to a preferred feature of the present invention, the nucleic acid is preferably introduced into a plant or plant cell by transformation. The term “transformation” is described in more detail in the “definitions” section herein.

In one embodiment the present invention extends to any plant cell or plant produced by any of the methods described herein, and to all plant parts and propagules thereof.

The present invention encompasses plants or parts thereof (including seeds) obtainable by the methods according to the present invention. The plants or plant parts or plant cells comprise a nucleic acid transgene encoding a NUG polypeptide as defined above, preferably in a genetic construct such as an expression cassette. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

In a further embodiment the invention extends to seeds comprising the expression cassettes of the invention, the genetic constructs of the invention, or the nucleic acids encoding the NUG and/or the NUG polypeptides as described above.

The invention also includes host cells containing an isolated nucleic acid encoding a NUG polypeptide as defined above. In one embodiment host cells according to the invention are plant cells, yeasts, bacteria or fungi. Host plants for the nucleic acids, construct, expression cassette or the vector used in the method according to the invention are, in principle, advantageously all plants which are capable of synthesizing the polypeptides used in the inventive method. In a particular embodiment the plant cells of the invention overexpress the nucleic acid molecule of the invention.

The methods of the invention are advantageously applicable to any plant, in particular to any plant as defined herein. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to an embodiment of the present invention, the plant is a crop plant. Examples of crop plants include but are not limited to chicory, carrot, cassava, trefoil, soybean, beet, sugar beet, sunflower, canola, alfalfa, rapeseed, linseed, cotton, tomato, potato and tobacco. According to another embodiment of the present invention, the plant is a monocotyledonous plant. Examples of monocotyledonous plants include sugarcane. According to another embodiment of the present invention, the plant is a cereal. Examples of cereals include rice, maize, wheat, barley, millet, rye, triticale, sorghum, emmer, spelt, einkorn, teff, milo and oats. In a particular embodiment the plants used in the methods of the invention are selected from the group consisting of maize, wheat, rice, soybean, cotton, oilseed rape including canola, sugarcane, sugar beet and alfalfa. Advantageously the methods of the invention are more efficient than the known methods, because the plants of the invention have increased yield and/or tolerance to an environmental stress compared to control plants used in comparable methods.

The invention also extends to harvestable parts of a plant such as, but not limited to seeds, leaves, fruits, flowers, stems, roots, rhizomes, tubers and bulbs, which harvestable parts comprise a recombinant nucleic acid encoding a NUG polypeptide. The invention furthermore relates to products derived or produced, preferably directly derived or produced, from a harvestable part of such a plant, such as dry pellets, meal or powders, oil, fat and fatty acids, starch or proteins.

The invention also includes methods for manufacturing a product comprising a) growing the plants of the invention and b) producing said product from or by the plants of the invention or parts thereof, including seeds. In a further embodiment the methods comprise the steps of a) growing the plants of the invention, b) removing the harvestable parts as described herein from the plants and c) producing said product from, or with the harvestable parts of plants according to the invention.

In one embodiment the products produced by the methods of the invention are plant products such as, but not limited to, a foodstuff, feedstuff, a food supplement, feed supplement, fiber, cosmetic or pharmaceutical. In another embodiment the methods for production are used to make agricultural products such as, but not limited to, plant extracts, proteins, amino acids, carbohydrates, fats, oils, polymers, vitamins, and the like.

In yet another embodiment, the polynucleotides or the polypeptides of the invention are comprised in an agricultural product. In a particular embodiment the nucleic acid sequences and protein sequences of the invention may be used as product markers, for example where an agricultural product was produced by the methods of the invention. Such a marker can be used to identify a product to have been produced by an advantageous process resulting not only in a greater efficiency of the process but also improved quality of the product due to increased quality of the plant material and harvestable parts used in the process. Such markers can be detected by a variety of methods known in the art, for example but not limited to PCR based methods for nucleic acid detection or antibody based methods for protein detection.

The present invention also encompasses use of nucleic acids encoding NUG polypeptides as described herein and use of these NUG polypeptides in enhancing any of the aforementioned yield-related traits in plants. For example, nucleic acids encoding NUG polypeptide described herein, or the NUG polypeptides themselves, may find use in breeding programmes in which a DNA marker is identified which may be genetically linked to a NUG polypeptide-encoding gene. The nucleic acids/genes, or the NUG polypeptides themselves may be used to define a molecular marker. This DNA or protein marker may then be used in breeding programmes to select plants having enhanced yield-related traits as defined herein in the methods of the invention. Furthermore, allelic variants of a NUG polypeptide-encoding nucleic acid/gene may find use in marker-assisted breeding programmes. Nucleic acids encoding NUG polypeptides may also be used as probes for genetically and physically mapping the genes that they are a part of, and as markers for traits linked to those genes. Such information may be useful in plant breeding in order to develop lines with desired phenotypes.

### 2. NAC1 and NAC5

According to a second aspect of the present invention, there is provided a method for enhancing yield-related traits in plants grown under abiotic stress conditions, comprising modulating expression in a plant of a nucleic acid encoding a NAC1 or NAC5 polypeptide.

In a particular embodiment, where plants are grown under abiotic stress conditions, expression of the NAC1 or NAC5-encoding nucleic acid is driven by a tissue-specific promoter, preferably by a root-specific promoter.

In a further embodiment, the enhanced yield-related traits comprise increased seed yield and/or modified root architecture.

According to a further aspect of the present invention, there is provided a method for producing plants having enhanced yield-related traits relative to control plants, comprising the steps of modulating expression in plants grown under abiotic stress of a nucleic acid encoding a NAC1 or NAC5 polypeptide and optionally selecting for plants having enhanced yield-related traits.

According to a further aspect of the present invention, there is provided a method for conferring abiotic stress tolerance in plants comprising modulating expression in a plant of a nucleic acid encoding a NAC1 or NAC5 polypeptide.

In the context of the invention concerning NAC1 and NAC5, any reference to a “protein useful in the methods of the invention” is taken to mean a NAC1 or NAC5 polypeptide as defined herein. Any reference hereinafter to a “nucleic acid useful in the methods of the invention” is taken to mean a nucleic acid capable of encoding a NAC1 or NAC5 polypeptide. The nucleic acid to be introduced into a plant (and therefore useful in performing the methods of the invention) is any nucleic acid encoding the type of protein which will now be described, hereinafter also named “NAC1 nucleic acid” or “NAC1 gene” or “NAC5 nucleic acid” or NAC5 gene”.

A “NAC1 polypeptide” or a “NAC5 polypeptide” as defined herein refers to any polypeptide comprising any one or more of the motifs described below.

A “NAC1 gene” or a “NAC5 gene” as defined herein refers to any nucleic acid encoding a NAC1 polypeptide or a NAC5 polypeptide as defined herein.

Motif I: KIDLDIIQELD, or a motif having in increasing order of preference at least 50%, 60%, 70%, 80% or 90% sequence identity to the sequence of Motif I.

Motif I is preferably K/P/R/G I/S/M D/A/E/Q L/I/V D I/V/F I Q/V/R/K E/D L/I/V D.

Motif II: CKYGXGHGGDEQTEW, or a motif having in increasing order of preference at least 50%, 60%, 70%, 80% or 90% sequence identity to the sequence of Motif II, where ‘X’ is taken to be any amino acid.

Motif II is preferably C K/R Y/L/I G XXX G/Y/N D/E E Q/R T/N/S EW, where ‘X’ is any amino acid.

Motif III: GWVVCRAFQKP, or a motif having in increasing order of preference at least 50%, 60%, 70%, 80% or 90% sequence identity to the sequence of Motif III.

Motif III is preferably GWVVCR A/V F X¹ K X², where ‘X¹’ and ‘X²’ may be any amino acid, preferably X¹ is Q/R/K, preferably X² is P/R/K.

Motif IV: PVPIIA, or a motif having in increasing order of preference at least 50%, 60%, 70%, 80% or 90% sequence identity to the sequence of Motif IV.

Motif IV is preferably A/P/S/N V/L/I/A P/S/D/V/Q V/I I A/T/G.

Motif V: NGSRPN, or a motif having in increasing order of preference at least 50%, 60%, 70%, 80% or 90% sequence identity to the sequence of Motif V.

Motif V is preferably N G/S S/Q/A/V RP N/S.

Motif VI: CRLYNKK, or a motif having in increasing order of preference at least 50%, 60%, 70%, 80% or 90% sequence identity to the sequence of Motif VI.

Motif VI is preferably C/Y R/K L/I Y/H/F N/K K K/N/C/S/T

Motif VII: NEWEKMQ, or a motif having in increasing order of preference at least 50%, 60%, 70%, 80% or 90% sequence identity to the sequence of Motif VII.

Motif VII is preferably N E/Q/T WEK M/V Q/R/K

Motif VIII: WGETRTPESE, or a motif having in increasing order of preference at least 50%, 60%, 70%, 80% or 90% sequence identity to the sequence Motif VIII.

Motif VIII is preferably WGE T/A RTPES E/D

Motif IX: VPKKESMDDA, or a motif having in increasing order of preference at least 50%, 60%, 70%, 80% or 90% sequence identity to the sequence of Motif IX.

Motif IX is preferably V/L PK K/E E S/R/A/V M/V/A/Q/R D/E D/E/L A/G/D

Motif X: SYDDIQGMYS, or a motif having in increasing order of preference at least 50%, 60%, 70%, 80% or 90% sequence identity to the sequence of Motif X.

Motif X is preferably S L/Y DD LII Q G/S L/M/P G/Y S/N.

Motif XI: DSMPRLHADSSCSE, or a motif having in increasing order of preference at least 50%, 60%, 70%, 80% or 90% sequence identity to the sequence of Motif XI.

Motif XI is preferably DS M/V/I P R/K L/I/A H T/A/S D/E SS C/G SE.

Each of motifs I to XI may comprise one or more conservative amino acid substitution at any position.

The NAC1 or NAC5 polypeptide may comprises at least 1 or at least 2 or at least 3 or at least 4 or at least 5 or at least 6 or at least 7 or at least 8 or at least 9 or at least 10 or at least 11 of the motifs defined above.

Further motifs present in NAC1 or NAC5 polypeptides may be identified using the MEME algorithm (Bailey and Elkan, Proceedings of the Second International Conference on Intelligent Systems for Molecular Biology, pp. 28-36, AAAI Press, Menlo Park, Calif., 1994) or using other methods or tools known in the art.

A preferred method for modulating (preferably, increasing) expression of a nucleic acid encoding a NAC1 or NAC5 polypeptide is by introducing and expressing in a plant a nucleic acid encoding a NAC1 or NAC5 polypeptide

According one aspect of the invention, there is provided a method for improving yield-related traits in plants and/or modifying root architecture relative to control plants, comprising modulating expression in a plant of a nucleic acid encoding a NAC1 or NAC5 polypeptide as defined herein.

Additionally or alternatively, the NAC1 or NAC5 polypeptide has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2 or SEQ ID NO: 4, provided that the homologous protein comprises any one or more of the conserved motifs as outlined above. In a particular embodiment the NAC1 polypeptide is represented by SEQ ID NO: 2. In a particular embodiment the NAC5 polypeptide is represented by SEQ ID NO: 4.

The overall sequence identity may be determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). In one embodiment the sequence identity level is determined by comparison of the polypeptide sequences over the entire length of the sequence of SEQ ID NO: 2 or SEQ ID NO: 4.

In another embodiment, the sequence identity level is determined by comparison of one or more conserved domains or motifs in SEQ ID NO: 2 or SEQ ID NO: 4 with corresponding conserved domains or motifs in other NAC1 and NAC5 polypeptides. Compared to overall sequence identity, the sequence identity will generally be higher when only conserved domains or motifs are considered. Preferably the motifs in a NAC1 or NAC5 polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one or more of the motifs represented by SEQ ID NO: 5 to SEQ ID NO: 15 (Motifs I to XI). The terms “domain”, “signature” and “motif” are as defined in the “definitions” section herein.

Preferably, the polypeptide sequence which when used in the construction of a phylogenetic tree, such as the given in Ooka et al., 2003 (DNA Research 10, 239-247), clusters with other NAC1 and NAC5 family members rather than with any other NAC.

Nucleic acids encoding NAC1 and NAC5 polypeptides, when expressed in rice according to the methods of the present invention as outlined in the Examples section herein, give plants grown under abiotic stress conditions enhanced yield related traits, in particular increased seed yield and/or modified root architecture. Another function of the nucleic acid sequences encoding NAC1 and NAC5 polypeptides is to confer information for synthesis of the NAC1 and NAC5 that increases yield or yield related traits as described herein, when such a nucleic acid sequence of the invention is transcribed and translated in a living plant cell.

The present invention is illustrated by transforming plants with the nucleic acid sequence represented by SEQ ID NO: 1, encoding the polypeptide sequence of SEQ ID NO: 2 and by transforming plants with the nucleic acid sequence represented by SEQ ID NO: 3 encoding the polypeptide of SEQ ID NO: 4. However, performance of the invention is not restricted to these sequences; the methods of the invention may advantageously be performed using any NAC1-encoding or NAC5-encoding nucleic acid or NAC1 or NAC5 polypeptide as defined herein. The term “NAC1” or “NAC1 polypeptide” as used herein also includes homologues as defined hereunder of SEQ ID NO: 2. The term “NAC5” or “NAC5 polypeptide” as used herein also includes homologues as defined hereunder of SEQ ID NO: 4.

Examples of nucleic acids encoding NAC1 and NAC5 polypeptides are given in Table C herein. Such nucleic acids are useful in performing the methods of the invention. The amino acid sequences given in Table C of the Examples section are example sequences of orthologues and paralogues of the NAC1 and NAC5 polypeptide represented by SEQ ID NO: 2 and SEQ ID NO: 4 respectively, the terms “orthologues” and “paralogues” being as defined herein. Further orthologues and paralogues may readily be identified by performing a so-called reciprocal blast search as described in the definitions section; where the query sequence is SEQ ID NO: 1, SEQ ID NO: 2, SEQ ID NO: 3 or SEQ ID NO: 4, the second BLAST (back-BLAST) would be against rice sequences.

Nucleic acid variants may also be useful in practising the methods of the invention. Examples of such variants include nucleic acids encoding homologues and derivatives of any one of the amino acid sequences given in Table C herein, the terms “homologue” and “derivative” being as defined herein. Also useful in the methods of the invention are nucleic acids encoding homologues and derivatives of orthologues or paralogues of any one of the amino acid sequences given in Table C of the Examples section. Homologues and derivatives useful in the methods of the present invention have substantially the same biological and functional activity as the unmodified protein from which they are derived. Further variants useful in practising the methods of the invention are variants in which codon usage is optimised or in which miRNA target sites are removed.

Further nucleic acid variants useful in practising the methods of the invention include portions of nucleic acids encoding NAC1 and NAC5 polypeptides, nucleic acids hybridising to nucleic acids encoding NAC1 or NAC5 polypeptides, splice variants of nucleic acids encoding NAC1 or NAC5 polypeptides, allelic variants of nucleic acids encoding NAC1 or NAC5 polypeptides and variants of nucleic acids encoding NAC1 or NAC5 polypeptides obtained by gene shuffling. The terms hybridising sequence, splice variant, allelic variant and gene shuffling are as described herein.

Nucleic acids encoding NAC1 or NAC5 polypeptides need not be full-length nucleic acids, since performance of the methods of the invention does not rely on the use of full-length nucleic acid sequences. According to the present invention, there is provided a method for enhancing yield-related traits in plants grown under abiotic stress conditions, comprising introducing and expressing in a plant a portion of a nucleic acid encoding any one of the proteins given in Table C herein, or a portion of a nucleic acid encoding an orthologue, paralogue or homologue of any of the amino acid sequences given in Table C.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a NAC1 or NAC5 polypeptide as defined herein or at least part thereof, and have substantially the same biological activity as the amino acid sequence given in Table C herein and encoded by the nucleic acid from which the portion is derived. Preferably, the portion is a portion of a nucleic acid encoding any one of the proteins given in Table C or is a portion of a nucleic acid encoding an orthologue or paralogue of any one of the amino acid sequences given in Table C. Preferably the portion is at least 500, 550, 600, 650, 700, 750, 800, 850, 900, 950, 1000 consecutive nucleotides in length, the consecutive nucleotides being of any one of the nucleic acid sequences given in Table C herein, or of a nucleic acid encoding an orthologue or paralogue of any one of the amino acid sequences given in Table C. Most preferably the portion is a portion of a nucleic acid encoding SEQ ID NO: 2 or SEQ ID NO: 4. Preferably, the portion encodes a fragment of an amino acid sequence which comprises one or more of motifs I to XI (SEQ ID NO: 5 to SEQ ID NO: 15) and/or has the same biological activity as a NAC1 or NAC5 and/or has at least 50%, 55%, 60%, 65%, 70%, 75%, 80%, 85%, 90%, 95% or more sequence identity to SEQ ID NO: 2 or to SEQ ID NO: 4.

Another nucleic acid variant useful in the methods of the invention is a nucleic acid capable of hybridising, under reduced or medium stringency conditions, preferably under stringent conditions, with the complement of a nucleic acid encoding a NAC1 or NAC5 polypeptide as defined herein, or with a portion as defined herein. According to the present invention, there is provided a method for enhancing yield-related traits in plants grown under abiotic stress conditions, comprising introducing and expressing in a plant a nucleic acid capable of hybridizing to a nucleic acid encoding any one of the proteins given in Table C herein, or to a nucleic acid encoding an orthologue, paralogue or homologue of any of the nucleic acid sequences given in Table C.

Hybridising sequences useful in the methods of the invention encode a NAC1 or NAC5 polypeptide as defined herein, having substantially the same biological activity as the amino acid sequence given in Table C encoded by the nucleic acid to which the hybridising sequence hybridises. Preferably, the hybridising sequence is capable of hybridising to the complement of a nucleic acid encoding any one of the proteins given in Table C herein, or to a portion of any of these sequences, a portion being as defined herein, or the hybridising sequence is capable of hybridising to the complement of a nucleic acid encoding an orthologue or paralogue of any one of the amino acid sequences given in Table C. Most preferably, the hybridising sequence is capable of hybridising to the complement of a nucleic acid encoding the polypeptide as represented by SEQ ID NO: 2 or SEQ ID NO: 4 or to a portion of either. In one embodiment, the hybridization conditions are medium stringency, preferably high stringency, as defined herein.

Preferably, the hybridising sequence encodes a polypeptide with an amino acid sequence comprising one or more of motifs I to XI (SEQ ID NO: 5 to SEQ ID NO: 15) and/or has the same biological activity as a NAC1 or NAC5 and/or has at least 50%, 55%, 60%, 65%, 70%, 75%, 80%, 85%, 90%, 95% or more sequence identity to SEQ ID NO: 2 or to SEQ ID NO: 4.

In another embodiment, there is provided a method for enhancing yield-related traits in plants grown under abiotic stress conditions, comprising introducing and expressing in a plant a splice variant or allelic variant of any one of a nucleic acid encoding any one of the proteins given in Table C herein or a splice variant or allelic variant of a nucleic acid encoding an orthologue, paralogue or homologue of any of the amino acid sequences given in Table C herein.

Preferred splice or allelic variants are splice or allelic variants of a nucleic acid encoding SEQ ID NO: 2 or SEQ ID NO: 4, or a splice or allelic variant of a nucleic acid encoding an orthologue or paralogue of SEQ ID NO: 2 or SEQ ID NO: 4. Preferably, the amino acid sequence encoded by the splice variant or allelic variant comprises one or more of motifs I to XI (SEQ ID NO: 5 to SEQ ID NO: 15) and/or has the same biological activity as a NAC1 or NAC5 and/or has at least 50%, 55%, 60%, 65%, 70%, 75%, 80%, 85%, 90%, 95% or more sequence identity to SEQ ID NO: 2 or to SEQ ID NO: 4.

According to a further embodiment, there is provided a method for enhancing yield-related traits in plants grown under abiotic stress conditions, comprising introducing and expressing in a plant an allelic variant or splice variant of a nucleic acid encoding any one of the proteins given in Table C herein, or comprising introducing and expressing in a plant an allelic variant or splice variant of a nucleic acid encoding an orthologue, paralogue or homologue of any of the amino acid sequences given in Table C herein.

The polypeptides encoded by allelic variants or splice variants useful in the methods of the present invention have substantially the same biological activity as the NAC1 polypeptide of SEQ ID NO: 2 or the NAC5 polypeptide of SEQ ID NO: 5 or of any of the amino acids depicted in Table C herein. Allelic variants exist in nature, and encompassed within the methods of the present invention is the use of these natural alleles. Preferably, the allelic variant or splice variant is a variant of a nucleic acid encoding SEQ ID NO: 2 or SEQ ID NO: 4 or a variant of a nucleic acid encoding an orthologue or paralogue of SEQ ID NO: 2 or SEQ ID NO: 4. Preferably, the amino acid sequence encoded by the allelic variant or splice variant comprises one or more of motifs I to XI (SEQ ID NO: 5 to SEQ ID NO: 15) and/or has the same biological activity as a NAC1 or NAC5 and/or has at least 50%, 55%, 60%, 65%, 70%, 75%, 80%, 85%, 90%, 95% or more sequence identity to SEQ ID NO: 2 or to SEQ ID NO: 4.

In yet another embodiment, there is provided a method for enhancing yield-related traits in plants grown under abiotic stress conditions, comprising introducing and expressing in a plant a variant of a nucleic acid encoding any one of the proteins given in Table C herein, or comprising introducing and expressing in a plant a variant of a nucleic acid encoding an orthologue, paralogue or homologue of any of the amino acid sequences given in Table C, which variant nucleic acid is obtained by gene shuffling.

Preferably, the amino acid sequence encoded by the variant nucleic acid obtained by gene shuffling comprises one or more of motifs I to XI (SEQ ID NO: 5 to SEQ ID NO: 15) and/or has the same biological activity as a NAC1 or NAC5 and/or has at least 50%, 55%, 60%, 65%, 70%, 75%, 80%, 85%, 90%, 95% or more sequence identity to SEQ ID NO: 2 or to SEQ ID NO: 4.

Furthermore, nucleic acid variants may also be obtained by site-directed mutagenesis. Several methods are available to achieve site-directed mutagenesis, the most common being PCR based methods (Current Protocols in Molecular Biology. Wiley Eds.). NCG polypeptides differing from the sequence of SEQ ID NO: 2 or SEQ ID NO: 4 by one or several amino acids (substitution(s), insertion(s) and/or deletion(s) as defined herein) may equally be useful to increase the yield of plants in the methods and constructs and plants of the invention.

Nucleic acids encoding a NAC1 or NAC5 polypeptide may be derived from any natural or artificial source. The nucleic acid may be modified from its native form in composition and/or genomic environment through deliberate human manipulation. Preferably the NAC1 or NAC5 polypeptide-encoding nucleic acid is from a plant, further preferably from a monocotyledonous plant, more preferably from the family Poaceae, most preferably the nucleic acid is from Oryza sativa.

In another embodiment the present invention extends to recombinant chromosomal DNA comprising a nucleic acid sequence useful in the methods of the invention, wherein said nucleic acid is present in the chromosomal DNA as a result of recombinant methods, but is not in its natural genetic environment. In a further embodiment the recombinant chromosomal DNA of the invention is comprised in a plant cell.

Performance of the methods of the invention gives plants having enhanced yield-related traits. In particular, performance of the methods of the invention gives plants having increased seed or grain yield and/or modified root architecture. The term “seed yield” is described in more detail in the “definitions” section herein. The term “modified root architecture” as defined herein preferably comprises or is due to an increase or change in any one or more of the following: an increase in root biomass in the form of fresh weight or dry weight, increased number of roots, increased root diameter, enlarged roots, enlarged stele, enlarged aerenchyma, increased aerenchyma formation, enlarged cortex, enlarged cortical cells, enlarged xylem, modified branching, improved penetration ability, enlarged epidermis, increase in the ratio of roots to shoots.

The present invention therefore provides a method for increasing seed yield and/or modified root architecture relative to control plants, which method comprises modulating expression in a plant grown under abiotic stress conditions of a nucleic acid encoding a NAC1 and NAC5 polypeptide.

The present invention also provides a method for increasing abiotic stress tolerance in plants relative to control plants, which method comprises modulating expression in a plant grown under abiotic stress conditions of a nucleic acid encoding a NAC1 and NAC5 polypeptide.

According to a preferred feature of the present invention, performance of the methods of the invention gives plants grown under abiotic stress conditions increased growth rate relative to control plants. Therefore, according to the present invention, there is provided a method for increasing the growth rate of plants, which method comprises modulating expression of a nucleic acid encoding a NAC1 or NAC5 polypeptide in a plant grown under abiotic stress conditions.

Performance of the methods of the invention in plants during their vegetative growth stage, which plants are grown under non-stress conditions or under mild drought conditions, gives enhanced yield-related traits and/or modified root architecture relative to control plants grown under comparable conditions. Therefore, according to the present invention, there is provided a method for enhancing yield-related traits and/or modifying root architecture in plants during their vegetative growth phase and grown under non-stress conditions or under mild drought conditions, which method comprises modulating expression in said plants of a nucleic acid encoding a NAC1 or NAC5 polypeptide.

Performance of the methods of the invention gives plants grown under conditions of drought, enhanced yield-related traits and/or modified root architecture relative to control plants grown under comparable conditions. Therefore, according to the present invention, there is provided a method for enhancing yield-related traits and/or modifying root architecture in plants grown under conditions of drought, which method comprises modulating expression in a plant of a nucleic acid encoding a NAC1 or NAC5 polypeptide under the control of a tissue-specific promoter, preferably a root-specific promoter.

Under normal or non-stress growth conditions rice plants expressing a NAC1-encoding nucleic acid sequence when expressed under the control of a constitutive promoter and when expressed under the control of a root-specific promoter gave increased seed yield. In comparison, significantly increased levels of seed or grain yield were obtained under drought conditions in plants expressing a NAC1-encoding nucleic acid under the control of a root-specific promoter. In contrast, there was no noticeable difference in the seed or grain yield of plants grown under drought stress and expressing a NAC1-encoding nucleic acid sequence under the control of a constitutive promoter compared non transgenic controls.

In the case of NAC5, plants expressing a NAC5-encoding nucleic acid under the control of a root specific promoter and plants expressing a NAC5-encoding nucleic acid under the control of a constitutive promoter showed increased tolerance to drought and high salinity during the vegetative growth phase. Under normal, non-stress growth conditions these plants showed increased seed or grain yield. However, under drought stress, plants expressing a NAC5 under the control of a root-specific promoter showed significantly increased seed or grain yield, whereas plants expressing a NAC5 under the control of a constitutive promoter showed a similar or reduced yield compared to non-transgenic control plants.

Performance of the methods of the invention gives plants grown under conditions of nutrient deficiency, particularly under conditions of nitrogen deficiency, enhanced yield-related traits and/or modified root architecture relative to control plants grown under comparable conditions. Therefore, according to the present invention, there is provided a method for enhancing yield-related traits and/or modifying root architecture in plants grown under conditions of nutrient deficiency, which method comprises modulating expression in a plant of a nucleic acid encoding a NAC1 or NAC5 polypeptide.

Performance of the methods of the invention gives plants grown under conditions of salt stress, enhanced yield-related traits and/or modified root architecture relative to control plants grown under comparable conditions. Therefore, according to the present invention, there is provided a method for enhancing yield-related traits and/or modifying root architecture in plants grown under conditions of salt stress, which method comprises modulating expression in a plant of a nucleic acid encoding a NAC1 or NAC5 polypeptide.

The invention also provides genetic constructs and vectors to facilitate introduction and/or expression in plants of nucleic acids encoding NAC1 or NAC5 polypeptides. The gene constructs may be inserted into vectors, which may be commercially available, suitable for transforming into plants or host cells and suitable for expression of the gene of interest in the transformed cells. The invention also provides use of a gene construct as defined herein in the methods of the invention.

More specifically, the present invention provides a construct comprising:
(a) a nucleic acid encoding a NAC1 or NAC5 polypeptide as defined above;
(b) one or more control sequences capable of driving expression of the nucleic acid sequence of (a); and optionally
(c) a transcription termination sequence.

Preferably, the nucleic acid encoding a NAC1 or NAC5 polypeptide is as defined above. The term “control sequence” and “termination sequence” are as defined herein.

The genetic construct of the invention may be comprised in a host cell, plant cell, seed, agricultural product or plant. Plants or host cells are transformed with a genetic construct such as a vector or an expression cassette comprising any of the nucleic acids described above. Thus the invention furthermore provides plants or host cells transformed with a construct as described above. In particular, the invention provides plants transformed with a construct as described above, which plants have increased yield-related traits as described herein.

In one embodiment the genetic construct of the invention confers increased yield or yield related traits(s) to a plant when it has been introduced into said plant, which plant expresses the nucleic acid encoding the NAC1 or NAC5 polypeptide comprised in the genetic construct. In another embodiment the genetic construct of the invention confers increased yield or yield related traits(s) to a plant comprising plant cells in which the construct has been introduced, which plant cells express the nucleic acid encoding the NAC1 or NAC5 comprised in the genetic construct.

The skilled artisan is well aware of the genetic elements that must be present on the genetic construct in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence during the vegetative growth phase of a plant. Preferably the promoter is of plant origin. See the “Definitions” section herein for definitions of the various promoter types.

A particularly preferred promoter for use in the methods of the invention is a root-specific promoter. The root-specific promoter is preferably an RCc3 promoter (Plant Mol Biol. 1995 January; 27(2):237-48) or a promoter of substantially the same strength and having substantially the same expression pattern (a functionally equivalent promoter), more preferably the RCc3 promoter is from rice, further preferably the RCc3 promoter is represented by a nucleic acid sequence substantially similar to SEQ ID NO: 21, most preferably the promoter is as represented by SEQ ID NO: 21. Examples of other root-specific promoters which may also be used to perform the methods of the invention are shown in Table 2b in the “Definitions” section.

A constitutive promoter may also be used in plants grown under stress or non-stress conditions, particularly during the vegetative growth phase of a plant. A constitutive promoter may also be used in plants grown under substantially non-stress conditions and expressing a NAC1 or NAC5-encoding nucleic acid. The constitutive promoter is preferably a ubiquitous constitutive promoter of medium strength. More preferably it is a plant derived promoter, e.g. a promoter of plant chromosomal origin, such as a GOS2 promoter or a promoter of substantially the same strength and having substantially the same expression pattern (a functionally equivalent promoter), more preferably the promoter is the promoter GOS2 promoter from rice. Further preferably the constitutive promoter is represented by a nucleic acid sequence substantially similar to SEQ ID NO: 20, most preferably the constitutive promoter is as represented by SEQ ID NO: 20. See the “Definitions” section herein for further examples of constitutive promoters.

It should be clear that the applicability of the present invention is not restricted to the NAC1 or NAC5 polypeptide-encoding nucleic acid represented by SEQ ID NO: 1 or SEQ ID NO: 3, nor is the applicability of the invention restricted to the rice GOS2 or RCc3 promoters for driving expression of a NAC1 or NAC5 in a plant.

Optionally, one or more terminator sequences may be used in the construct introduced into a plant. Those skilled in the art will be aware of terminator sequences that may be suitable for use in performing the invention.

Preferably, the construct comprises an expression cassette comprising an RCc3 promoter operably linked to the nucleic acid encoding the NAC1 or NAC5 polypeptide. More preferably, the construct furthermore comprises a zein terminator (t-zein) linked to the 3′ end of the NAC1 or NAC5 coding sequence. Furthermore, one or more sequences encoding selectable markers may be present on the construct introduced into a plant.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a nucleic acid encoding a NAC1 or NAC5 polypeptide is by introducing and expressing in a plant a nucleic acid encoding a NAC1 or NAC5 polypeptide; however the effects of performing the method, i.e. enhancing yield-related traits and/or modifying root architecture may also be achieved using other well-known techniques, including but not limited to T-DNA activation tagging, TILLING, homologous recombination. A description of these techniques is provided in the definitions section.

The invention also provides a method for the production of transgenic plants having enhanced yield-related traits and/or modified root architecture relative to control plants, comprising introduction and expression in a plant of any nucleic acid encoding a NAC1 or NAC5 polypeptide as defined herein.

More specifically, the present invention provides a method for the production of transgenic plants having enhanced yield-related traits, particularly increased seed yield and/or modified root architecture, which method comprises:
(i) introducing and expressing in a plant or plant cell a NAC1 or NAC5 polypeptide-encoding nucleic acid or a genetic construct comprising a NAC1 or NAC5 polypeptide-encoding nucleic acid; and
(ii) cultivating the plant cell under abiotic stress conditions.

The nucleic acid of (i) may be any of the nucleic acids capable of encoding a NAC1 or NAC5 polypeptide as defined herein.

Cultivating the plant cell, may or may not include regeneration and/or growth to maturity. Accordingly, in a particular embodiment of the invention, the plant cell transformed by the method according to the invention is regenerable into a transformed plant. In another particular embodiment, the plant cell transformed by the method according to the invention is not regenerable into a transformed plant, i.e. cells that are not capable to regenerate into a plant using cell culture techniques known in the art. While plants cells generally have the characteristic of totipotency, some plant cells cannot be used to regenerate or propagate intact plants from said cells. In one embodiment of the invention the plant cells of the invention are such cells. In another embodiment the plant cells of the invention are plant cells that do not sustain themselves in an autotrophic way.

The nucleic acid may be introduced directly into a plant cell or into the plant itself (including introduction into a tissue, organ or any other part of a plant). According to a preferred feature of the present invention, the nucleic acid is preferably introduced into a plant or plant cell by transformation. The term “transformation” is described in more detail in the “definitions” section herein.

In one embodiment the present invention extends to any plant cell or plant produced by any of the methods described herein, and to all plant parts and propagules thereof.

The present invention encompasses plants or parts thereof (including seeds) obtainable by the methods according to the present invention. The plants or plant parts or plant cells comprise a nucleic acid transgene encoding a NAC1 or NAC5 polypeptide as defined above, preferably in a genetic construct such as an expression cassette. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

In a further embodiment the invention extends to seeds comprising the expression cassettes of the invention, the genetic constructs of the invention, or the nucleic acids encoding the NAC1 or NAC5 and/or the NAC1 or NAC5 polypeptides as described above.

The invention also includes host cells containing an isolated nucleic acid encoding a NAC1 or NAC5 polypeptide as defined above. In one embodiment host cells according to the invention are plant cells, yeasts, bacteria or fungi. Host plants for the nucleic acids, construct, expression cassette or the vector used in the method according to the invention are, in principle, advantageously all plants which are capable of synthesizing the polypeptides used in the inventive method. In a particular embodiment the plant cells of the invention overexpress the nucleic acid molecule of the invention.

The methods of the invention are advantageously applicable to any plant, in particular to any plant as defined herein. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to an embodiment of the present invention, the plant is a crop plant. Examples of crop plants include but are not limited to chicory, carrot, cassava, trefoil, soybean, beet, sugar beet, sunflower, canola, alfalfa, rapeseed, linseed, cotton, tomato, potato and tobacco. According to another embodiment of the present invention, the plant is a monocotyledonous plant. Examples of monocotyledonous plants include sugarcane. According to another embodiment of the present invention, the plant is a cereal. Examples of cereals include rice, maize, wheat, barley, millet, rye, triticale, sorghum, emmer, spelt, einkorn, teff, milo and oats. In a particular embodiment the plants used in the methods of the invention are selected from the group consisting of maize, wheat, rice, soybean, cotton, oilseed rape including canola, sugarcane, sugar beet and alfalfa. Advantageously the methods of the invention are more efficient than the known methods, because the plants of the invention have increased yield and/or tolerance to an environmental stress compared to control plants used in comparable methods.

The invention also extends to harvestable parts of a plant such as, but not limited to seeds, leaves, fruits, flowers, stems, roots, rhizomes, tubers and bulbs, which harvestable parts comprise a recombinant nucleic acid encoding a NAC1 or NAC5 polypeptide. The invention furthermore relates to products derived or produced, preferably directly derived or produced, from a harvestable part of such a plant, such as dry pellets, meal or powders, oil, fat and fatty acids, starch or proteins.

The invention also includes methods for manufacturing a product comprising a) growing the plants of the invention and b) producing said product from or by the plants of the invention or parts thereof, including seeds. In a further embodiment the methods comprise the steps of a) growing the plants of the invention, b) removing the harvestable parts as described herein from the plants and c) producing said product from, or with the harvestable parts of plants according to the invention.

In one embodiment the products produced by the methods of the invention are plant products such as, but not limited to, a foodstuff, feedstuff, a food supplement, feed supplement, fiber, cosmetic or pharmaceutical. In another embodiment the methods for production are used to make agricultural products such as, but not limited to, plant extracts, proteins, amino acids, carbohydrates, fats, oils, polymers, vitamins, and the like.

In yet another embodiment the polynucleotides or the polypeptides of the invention are comprised in an agricultural product. In a particular embodiment the nucleic acid sequences and protein sequences of the invention may be used as product markers, for example where an agricultural product was produced by the methods of the invention. Such a marker can be used to identify a product to have been produced by an advantageous process resulting not only in a greater efficiency of the process but also improved quality of the product due to increased quality of the plant material and harvestable parts used in the process. Such markers can be detected by a variety of methods known in the art, for example but not limited to PCR based methods for nucleic acid detection or antibody based methods for protein detection.

The present invention also encompasses use of nucleic acids encoding NAC1 or NAC5 polypeptides as described herein and use of these NAC1 or NAC5 polypeptides in enhancing any of the aforementioned yield-related traits or in modifying root architecture in plants. For example, nucleic acids encoding NAC1 or NAC5 polypeptide described herein, or the NAC1 or NAC5 polypeptides themselves, may find use in breeding programmes in which a DNA marker is identified which may be genetically linked to a NAC1 or NAC5 polypeptide-encoding gene. The nucleic acids/genes, or the NAC1 or NAC5 polypeptides themselves may be used to define a molecular marker. This DNA or protein marker may then be used in breeding programmes to select plants having enhanced yield-related traits or modified root architecture as defined herein in the methods of the invention. Furthermore, allelic variants of a NAC1 or NAC5 polypeptide-encoding nucleic acid/gene may find use in marker-assisted breeding programmes. Nucleic acids encoding NAC1 or NAC5 polypeptides may also be used as probes for genetically and physically mapping the genes that they are a part of, and as markers for traits linked to those genes. Such information may be useful in plant breeding in order to develop lines with desired phenotypes.

Furthermore, the present invention relates to the following specific embodiments.
A: A method for enhancing yield-related traits in plants relative to control plants, comprising modulating expression in a plant of a NAC up-regulated gene (NUG) encoding any one of the polypeptides given in Table A or Table B or a homologue thereof.
B: A method for enhancing yield-related traits and/or for modifying root architecture in plants grown under abiotic stress, comprising modulating expression in a plant of nucleic acid encoding a NAC1 or NAC5 polypeptide or homologue therefore, which nucleic acid is operably linked to a tissue-specific promoter.
C: Method according to embodiment A or embodiment B, wherein said modulated expression is effected by introducing and expressing in a plant a nucleic acid encoding a NUG, NAC1 or NAC5 polypeptide or a homologue thereof.
D: Method according to embodiment A, wherein said enhanced yield-related traits comprise increased yield and/or biomass relative to control plants.
E: Method according to embodiment B, wherein said enhanced yield-related traits comprise increased seed or grain yield and/or wherein said modified root architecture comprises or is due to an increase or change in any one or more of the following: an increase in root biomass in the form of fresh weight or dry weight, increased number of roots, increased root diameter, enlarged roots, enlarged stele, enlarged aerenchyma, increased aerenchyma formation, enlarged cortex, enlarged cortical cells, enlarged xylem, modified branching, improved penetration ability, enlarged epidermis, increase in the ratio of roots to shoots.
F: Method according to any one of embodiments A or C to E, wherein said enhanced yield-related traits are obtained under non-stress conditions.
G: Method according to any one of embodiments A to F, wherein said enhanced yield-related traits are obtained under conditions of drought stress, salt stress or nitrogen deficiency.
H: Method according to any one of embodiments B to G, wherein said NAC1 or NAC5 polypeptide comprises one or more of the motifs represented by SEQ ID NO: 5 to SEQ ID NO: 15.
I: Method according to any one of embodiments A to H, wherein said nucleic acid encoding a NUG, NAC1 or NAC5 is of plant origin, preferably from a monocotyledonous plant, further preferably from the family Poaceae, more preferably from the genus Oryza, most preferably from Oryza sativa.
J: Method according to any one of embodiments A to I, wherein said nucleic acid encoding a NUG, NAC1 or NAC5 encodes any one of the polypeptides listed in Table A, Table B or Table C or is a portion of such a nucleic acid, or a nucleic acid capable of hybridising with such a nucleic acid.
K: Method according to any one of embodiments A to J, wherein said nucleic acid sequence encodes an orthologue or paralogue of any of the polypeptides given in Table A, Table B or Table C.
L: Method according to any one of embodiments A to K, wherein said nucleic acid encodes the NAC1 polypeptide represented by SEQ ID NO: 2.
M: Method according to any one of embodiments A to L, wherein said nucleic acid encodes the NAC5 polypeptide represented by SEQ ID NO: 4.
N: Method according to any one of embodiments A and C to M, wherein said nucleic acid is operably linked to a constitutive promoter of plant origin, preferably to a medium strength constitutive promoter of plant origin, more preferably to a GOS2 promoter, most preferably to a GOS2 promoter from rice.
O: Method according to any one of embodiments B to M, wherein said tissue specific promoter is a root-specific promoter, preferably an RCc3 promoter, further preferably an RCc3 promoter from rice.
P: Plant, or part thereof, or plant cell, obtainable by a method according to any one of embodiments A to O, wherein said plant, plant part or plant cell comprises a recombinant nucleic acid encoding a NUG, NAC1 or NAC5 polypeptide as given in Table A, Table B or Table C or a homologue, paralogue or orthologue thereof.
Q: Construct comprising:
   (i) nucleic acid encoding an NUG, NAC1, NAC5 as given in Table A, Table B or Table C or a homologue, paralogue or orthologue thereof;
   (ii) one or more control sequences capable of driving expression of the nucleic acid sequence of (i); and optionally
   (iii) a transcription termination sequence.
R: Construct according to embodiment Q, wherein said nucleic acid is operably linked to a constitutive promoter of plant origin, preferably to a medium strength constitutive promoter of plant origin, more preferably to a GOS2 promoter, most preferably to a GOS2 promoter from rice.
S: Construct according to embodiment Q, wherein said nucleic acid is operably linked to a tissue specific promoter, preferably to a root-specific promoter, preferably to an RCc3 promoter, further preferably an RCc3 promoter from rice.
T: Use of a construct according to any one of embodiments Q to S in a method for making plants having enhanced yield-related traits, preferably increased seed yield and/or increased biomass and/or modified root architecture relative to control plants.
U: Plant, plant part or plant cell transformed with a construct according to any one of embodiments Q to S.
V: Method for the production of a transgenic plant having enhanced yield-related traits relative to control plants, preferably increased and/or increased seed yield and/or increased biomass relative to control plants, comprising:
   (i) introducing and expressing in a plant cell or plant a nucleic acid encoding a NUG polypeptide as given in Table A or Table B or a homologue, paralogue or orthologue thereof; and
   (ii) cultivating said plant cell or plant of (i) under conditions promoting plant growth and development; or
   (iii) introducing and expressing in a plant cell or plant a nucleic acid encoding a NAC1 or NAC5 polypeptide as given in Table C or a homologue, paralogue or orthologue thereof, which nucleic acid is operably linked to a tissue-specific promoter; and
   (iv) cultivating said plant cell or plant from step (iii) under abiotic stress conditions, wherein said plants have increased seed yield and modified root architecture.
W: Transgenic plant having enhanced yield-related traits relative to control plants resulting from modulated expression of a nucleic acid encoding an NUG, NAC1 or NAC5 polypeptide as given in Table A, Table B or Table C or a homologue, paralogue or orthologue thereof.
X: Transgenic plant according to embodiment P, U or W or a transgenic plant cell derived therefrom, wherein said plant is a crop plant, such as beet, sugarbeet or alfalfa; or a monocotyledonous plant such as sugarcane; or a cereal, such as rice, maize, wheat, barley, millet, rye, triticale, sorghum, emmer, spelt, einkorn, teff, milo or oats.
Y: Harvestable parts of a plant according to embodiment X, wherein said harvestable parts are preferably root biomass and/or seeds.
Z: Products derived from a plant according to embodiment X and/or from harvestable parts of a plant according to embodiment Y.
A′: Use of a nucleic acid encoding an NUG, NAC1 or NAC5 polypeptide as given in Table A, Table B or Table C or a homologue, paralogue or orthologue thereof for enhancing yield-related traits in plants relative to control plants.
B′ A method for manufacturing a product, comprising the steps of growing the plants according to embodiment P, U, W or X and producing said product from or by said plants, or parts thereof, including seeds.

### DEFINITIONS

The following definitions will be used throughout the present application. The section captions and headings in this application are for convenience and reference purpose only and should not affect in any way the meaning or interpretation of this application. The technical terms and expressions used within the scope of this application are generally to be given the meaning commonly applied to them in the pertinent art of plant biology, molecular biology, bioinformatics and plant breeding. All of the following term definitions apply to the complete content of this application. The term “essentially”, “about”, “approximately” and the like in connection with an attribute or a value, particularly also define exactly the attribute or exactly the value, respectively. The term “about” in the context of a given numeric value or range relates in particular to a value or range that is within 20%, within 10%, or within 5% of the value or range given.

### Peptide(s)/Protein(s)

The terms “peptides”, “oligopeptides”, “polypeptide” and “protein” are used interchangeably herein and refer to amino acids in a polymeric form of any length, linked together by peptide bonds, unless mentioned herein otherwise.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

Orthologues and paralogues are two different forms of homologues and encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

A “deletion” refers to removal of one or more amino acids from a protein.

An “insertion” refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A “substitution” refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols (see Current Protocols in Molecular Biology, John Wiley & Sons, N.Y. (1989 and yearly updates)).

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Domain, Motif/Consensus Sequence/Signature

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4: 29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

### Reciprocal BLAST

Typically, this involves a first BLAST involving BLASTing a query sequence (for example using any of the sequences listed in Table A of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting NCGnt (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:

1) DNA-DNA Hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]⁻¹−0.61×% formamide

### 2) DNA-RNA or RNA-RNA Hybrids:

Tₘ=79.8° C.+18.5(log₁₀[Na⁺]^{a})+0.58(%G/C^{b})+11.8(%G/C^{b})²×820/L^{c}

### 3) Oligo-DNA or Oligo-RNAs Hybrids:

For <20 nucleotides: Tₘ=2(Iₙ)

For 20-35 nucleotides: Tₘ=22+1.46(Iₙ)
^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; Iₙ,=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

“Alleles” or “allelic variants” are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Gene Shuffling/Directed Evolution

“Gene shuffling” or “directed evolution” consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Construct

Artificial DNA (such as but, not limited to plasmids or viral DNA) capable of replication in a host cell and used for introduction of a DNA sequence of interest into a host cell or host organism. Host cells of the invention may be any cell selected from bacterial cells, such as Escherichia coli or Agrobacterium species cells, yeast cells, fungal, algal or cyanobacterial cells or plant cells. The skilled artisan is well aware of the genetic elements that must be present on the genetic construct in order to successfully transform, select and propagate host cells containing the sequence of interest. The sequence of interest is operably linked to one or more control sequences (at least to a promoter) as described herein. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right NCGnt in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, |
| | WO 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 | Wu et al. Plant Mol. Biol. 11: 641-649, 1988 |
| histone | |
| Actin 2 | An et al, Plant J. 10(1); 107-121, 1996 |
| 34S FMV | Sanger et al., Plant. Mol. Biol., 14, 1990: 433-443 |
| Rubisco small | U.S. Pat. No. 4,962,028 |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. 12(20): |
| | 7831-7846 |
| V-ATPase | WO 01/14572 |
| Super promoter | WO 95/14098 |
| G-box proteins | WO 94/12015 |

### Ubiquitous Promoter

A “ubiquitous promoter” is active in substantially all tissues or cells of an organism.

### Developmentally-Regulated Promoter

A “developmentally-regulated promoter” is active during certain developmental stages or in parts of the plant that undergo developmental changes.

### Inducible Promoter

An “inducible promoter” has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An “organ-specific” or “tissue-specific promoter” is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 January; 27(2): 237-48 |
| Arabidopsis PHT1 | Koyama et al. J Biosci Bioeng. 2005 |
| | January; 99(1): 38-42.; Mudge et al. |
| | (2002, Plant J. 31: 341) |
| Medicago phosphate | Xiao et al., 2006, Plant Biol (Stuttg). |
| transporter | 2006 July; 8(4): 439-49 |
| Arabidopsis Pyk10 | Nitz et al. (2001) Plant Sci 161(2): 337-346 |
| root-expressible genes | Tingey et al., EMBO J. 6: 1, 1987. |
| tobacco auxin- | Van der Zaal et al., Plant Mol. Biol. 16, |
| inducible gene | 983, 1991. |
| β-tubulin | Oppenheimer, et al., Gene 63: 87, 1988. |
| tobacco root- | Conkling, et al., Plant Physiol. |
| specific genes | 93: 1203, 1990. |
| B. napus G1-3b gene | U.S. Pat. No. 5,401,836 |
| SbPRP1 | Suzuki et al., Plant Mol. Biol. 21: 109-119, |
| | 1993. |
| LRX1 | Baumberger et al. 2001, Genes & Dev. 15: 1128 |
| BTG-26 Brassica | U.S. 20050044585 |
| napus | |
| LeAMT1 (tomato) | Lauter et al. (1996, PNAS 3: 8139) |
| The LeNRT1-1 | Lauter et al. (1996, PNAS 3: 8139) |
| (tomato) | |
| class I patatin | Liu et al., Plant Mol. Biol. 17 (6): 1139-1154 |
| gene (potato) | |
| KDC1 (Daucus | Downey et al. (2000, J. Biol. Chem. 275: 39420) |
| carota) | |
| TobRB7 gene | W Song (1997) PhD Thesis, North Carolina State |
| | University, Raleigh, NC USA |
| OsRAB5a (rice) | Wang et al. 2002, Plant Sci. 163: 273 |
| ALF5 (Arabidopsis) | Diener et al. (2001, Plant Cell 13: 1625) |
| NRT2; 1Np (N. | Quesada et al. (1997, Plant Mol. Biol. 34: 265) |
| plumbaginifolia) | |

A “seed-specific promoter” is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are shown in Table 2c to Table 2f below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| Legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| Zein | Matzke et al Plant Mol Biol, 14(3): 323-32 1990 |
| napA | Stalberg et al, Planta 199: 515-519, 1996. |
| wheat LMW and HMW | Mol Gen Genet 216: 81-90, 1989; NAR 17: 461-2, 1989 |
| glutenin-1 | |
| wheat SPA | Albani et al, Plant Cell, 9: 171-184, 1997 |
| wheat α, β, γ-gliadins | EMBO J. 3: 1409-15, 1984 |
| barley ltr1 promoter | Diaz et al. (1995) Mol Gen Genet 248(5): 592-8 |
| barley B1, C, D, hordein | Theor Appl Gen 98: 1253-62, 1999; Plant J 4: 343-55, |
| | 1993; Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, |
| | 1996 |
| rice α-globulin REB/OHP-1 | Nakase et al. Plant Mol. Biol. 33: 513-522, 1997 |
| rice ADP-glucose pyrophos- | Trans Res 6: 157-68, 1997 |
| phorylase | |
| maize ESR gene family | Plant J 12: 235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor | unpublished |
| ITR1 (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

TABLE 2d
examples of endosperm-specific promoters

| Gene source | Reference |
|---|---|
| glutelin (rice) | Takaiwa et al. (1986) Mol Gen Genet 208: 15-22; |
| | Takaiwa et al. (1987) FEBS Letts. 221: 43-47 |
| Zein | Matzke et al., (1990) Plant Mol Biol 14(3): 323-32 |
| wheat LMW | Colot et al. (1989) Mol Gen Genet 216: 81-90, |
| and HMW | Anderson et al. (1989) NAR 17: 461-2 |
| glutenin-1 | |
| wheat SPA | Albani et al. (1997) Plant Cell 9: 171-184 |
| wheat gliadins | Rafalski et al. (1984) EMBO 3: 1409-15 |
| barley ltr1 | Diaz et al. (1995) Mol Gen Genet 248(5): 592-8 |
| promoter | |
| barley B1, C, D, | Cho et al. (1999) Theor Appl Genet 98: 1253-62; |
| hordein | Muller et al. (1993) Plant J 4: 343-55; |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin | Wu et al, (1998) Plant Cell Physiol 39(8) 885-889 |
| NRP33 | |
| rice globulin | Wu et al. (1998) Plant Cell Physiol 39(8) 885-889 |
| Glb-1 | |
| rice globulin | Nakase et al. (1997) Plant Molec Biol 33: 513-522 |
| REB/OHP-1 | |
| rice ADP-glucose | Russell et al. (1997) Trans Res 6: 157-68 |
| pyrophosphorylase | |
| maize ESR | Opsahl-Ferstad et al. (1997) Plant J 12: 235-46 |
| gene family | |
| sorghum kafirin | DeRose et al. (1996) Plant Mol Biol 32: 1029-35 |

TABLE 2e
Examples of embryo specific promoters:

| Gene source | Reference |
|---|---|
| rice OSH1 | Sato et al, Proc. Natl. Acad. Sci. USA, 93: 8117-8122, |
| | 1996 |
| KNOX | Postma-Haarsma et al, Plant Mol. Biol. 39: 257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase | Lanahan et al, Plant Cell 4: 203-211, 1992; |
| (Amy32b) | Skriver et al, Proc Natl Acad Sci USA 88: |
| | 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A “green tissue-specific promoter” as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g below.

TABLE 2g
Examples of green tissue-specific promoters

| Gene | Expression | Reference |
|---|---|---|
| Maize Orthophosphate | Leaf specific | Fukavama et al., Plant |
| dikinase | | Physiol. 2001 November; |
| | | 127(3): 1136-46 |
| Maize Phosphoenolpyruvate | Leaf specific | Kausch et al., Plant Mol |
| carboxylase | | Biol. 2001 January; |
| | | 45(1): 1-15 |
| Rice Phosphoenolpyruvate | Leaf specific | Liu et al., 2004 DNA |
| carboxylase | | Seq. 2004 August; |
| | | 15(4): 269-76 |
| Rice small subunit | Leaf specific | Nomura et al., Plant Mol |
| Rubisco | | Biol. 2000 September; |
| | | 44(1): 99-106 |
| rice beta expansin | Shoot specific | WO 2004/070039 |
| EXBP9 | | |
| Pigeonpea small subunit | Leaf specific | Panguluri et al., Indian |
| Rubisco | | J Exp Biol. 2005 April; |
| | | 43(4): 369-72 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| rice OSH1 | Shoot apical meristem, | Sato et al. (1996) |
| | from embryo globular | Proc. Natl. Acad. Sci. |
| | stage to seedling stage | USA, 93: 8117-8122 |
| Rice metallothionein | Meristem specific | BAD87835.1 |
| WAK1 & WAK 2 | Shoot and root apical | Wagner & Kohorn |
| | meristems, and in ex- | (2001) Plant Cell |
| | panding leaves and sepals | 13(2): 303-318 |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not present in, or originating from, the genome of said plant, or are present in the genome of said plant but not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

It shall further be noted that in the context of the present invention, the term “isolated nucleic acid” or “isolated polypeptide” may in some instances be considered as a synonym for a “recombinant nucleic acid” or a “recombinant polypeptide”, respectively and refers to a nucleic acid or polypeptide that is not located in its natural genetic environment and/or that has been modified by recombinant methods.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. For the purposes of this invention, the original unmodulated expression may also be absence of any expression. The term “modulating the activity” shall mean any change of the expression of the inventive nucleic acid sequences or encoded proteins, which leads to increased yield and/or increased growth of the plants. The expression can increase from zero (absence of, or immeasurable expression) to a certain amount, or can decrease from a certain amount to immeasurable small amounts or zero.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level. For the purposes of this invention, the original wild-type expression level might also be zero, i.e. absence of expression or immeasurable expression.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. MiRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art. Alternatively, a plant cell that cannot be regenerated into a plant may be chosen as host cell, i.e. the resulting transformed plant cell does not have the capacity to regenerate into a (whole) plant.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:1-9; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later NCGnt in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the abovementioned publications by S. D. Kung and R. Wu, Potrykus or Hofgen and Willmitzer. Alternatively, the genetically modified plant cells are non-regenerable into a whole plant.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

### T-DNA Activation Tagging

“T-DNA activation” tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

### Homologous Recombination

“Homologous recombination” allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9(10): 3077-84) but also for crop plants, for example rice (Terada et al. (2002) Nat Biotech 20(10): 1030-4; lida and Terada (2004) Curr Opin Biotech 15(2): 132-8), and approaches exist that are generally applicable regardless of the target organism (Miller et al, Nature Biotechnol. 25, 778-785, 2007).

### Yield Related Trait(s)

A “Yield related trait” is a trait or feature which is related to plant yield. Yield-related traits may comprise one or more of the following non-limitative list of features: early flowering time, yield, biomass, seed yield, early vigour, greenness index, growth rate, agronomic traits, such as e.g. tolerance to submergence (which leads to yield in rice), Water Use Efficiency (WUE), Nitrogen Use Efficiency (NUE), etc.

Reference herein to enhanced yield-related traits, relative to of control plants is taken to mean one or more of an increase in early vigour and/or in biomass (weight) of one or more parts of a plant, which may include (i) aboveground parts and preferably aboveground harvestable parts and/or (ii) parts below ground and preferably harvestable below ground. In particular, such harvestable parts are seeds.

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters.

The terms “yield” of a plant and “plant yield” are used interchangeably herein and are meant to refer to vegetative biomass such as root and/or shoot biomass, to reproductive organs, and/or to propagules such as seeds of that plant.

Flowers in maize are unisexual; male inflorescences (tassels) originate from the apical stem and female inflorescences (ears) arise from axillary bud apices. The female inflorescence produces pairs of spikelets on the surface of a central axis (cob). Each of the female spikelets encloses two fertile florets, one of them will usually mature into a maize kernel once fertilized. Hence a yield increase in maize may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate, which is the number of filled florets (i.e. florets containing seed) divided by the total number of florets and multiplied by 100), among others.

Inflorescences in rice plants are named panicles. The panicle bears spikelets, which are the basic units of the panicles, and which consist of a pedicel and a floret. The floret is borne on the pedicel and includes a flower that is covered by two protective glumes: a larger glume (the lemma) and a shorter glume (the palea). Hence, taking rice as an example, a yield increase may manifest itself as an increase in one or more of the following: number of plants per square meter, number of panicles per plant, panicle length, number of spikelets per panicle, number of flowers (or florets) per panicle; an increase in the seed filling rate which is the number of filled florets (i.e. florets containing seeds) divided by the total number of florets and multiplied by 100; an increase in thousand kernel weight, among others.

### Early Flowering Time

Plants having an “early flowering time” as used herein are plants which start to flower earlier than control plants. Hence this term refers to plants that show an earlier start of flowering. Flowering time of plants can be assessed by counting the number of days (“time to flower”) between sowing and the emergence of a first inflorescence. The “flowering time” of a plant can for instance be determined using the method as described in WO 2007/093444.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increased Growth Rate

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a mature seed up to the stage where the plant has produced mature seeds, similar to the starting material. This life cycle may be influenced by factors such as speed of germination, early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

### Stress Resistance

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Abiotic stresses may be due to drought or excess water, anaerobic stress, salt stress, chemical toxicity, oxidative stress and hot, cold or freezing temperatures.

“Biotic stresses” are typically those stresses caused by pathogens, such as bacteria, viruses, fungi, nematodes and insects.

The “abiotic stress” may be an osmotic stress caused by a water stress, e.g. due to drought, salt stress, or freezing stress. Abiotic stress may also be an oxidative stress or a cold stress. “Freezing stress” is intended to refer to stress due to freezing temperatures, i.e. temperatures at which available water molecules freeze and turn into ice. “Cold stress”, also called “chilling stress”, is intended to refer to cold temperatures, e.g. temperatures below 10°, or preferably below 5° C., but at which water molecules do not freeze. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

In particular, the methods of the present invention may be performed under non-stress conditions. In an example, the methods of the present invention may be performed under non-stress conditions such as mild drought to give plants having increased yield relative to control plants.

In another embodiment, the methods of the present invention may be performed under stress conditions.

In an example, the methods of the present invention may be performed under stress conditions such as drought to give plants having increased yield relative to control plants. In another example, the methods of the present invention may be performed under stress conditions such as nutrient deficiency to give plants having increased yield relative to control plants.

Nutrient deficiency may result from a lack of nutrients such as nitrogen, phosphates and other phosphorous-containing compounds, potassium, calcium, magnesium, manganese, iron and boron, amongst others.

In yet another example, the methods of the present invention may be performed under stress conditions such as salt stress to give plants having increased yield relative to control plants. The term salt stress is not restricted to common salt (NaCl), but may be any one or more of: NaCl, KCl, LiCl, MgCl₂, CaCl₂, amongst others.

In yet another example, the methods of the present invention may be performed under stress conditions such as cold stress or freezing stress to give plants having increased yield relative to control plants.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following:
a) an increase in seed biomass (total seed weight) which may be on an individual seed basis and/or per plant and/or per square meter;
b) increased number of flowers per plant;
c) increased number of seeds;
d) increased seed filling rate (which is expressed as the ratio between the number of filled florets divided by the total number of florets);
e) increased harvest index, which is expressed as a ratio of the yield of harvestable parts, such as seeds, divided by the biomass of aboveground plant parts; and
f) increased thousand kernel weight (TKW), which is extrapolated from the number of seeds counted and their total weight. An increased TKW may result from an increased seed size and/or seed weight, and may also result from an increase in embryo and/or endosperm size.

The terms “filled florets” and “filled seeds” may be considered synonyms.

An increase in seed yield may also be manifested as an increase in seed size and/or seed volume. Furthermore, an increase in seed yield may also manifest itself as an increase in seed area and/or seed length and/or seed width and/or seed perimeter.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Biomass

The term “biomass” as used herein is intended to refer to the total weight of a plant. Within the definition of biomass, a distinction may be made between the biomass of one or more parts of a plant, which may include any one or more of the following:
aboveground parts such as but not limited to shoot biomass, seed biomass, leaf biomass, etc.;
aboveground harvestable parts such as but not limited to shoot biomass, seed biomass, leaf biomass, etc.;
parts below ground, such as but not limited to root biomass, tubers, bulbs, etc.;
harvestable parts below ground, such as but not limited to root biomass, tubers, bulbs, etc.;
harvestable parts partially below ground such as but not limited to beets and other hypocotyl areas of a plant, rhizomes, stolons or creeping rootstalks;
vegetative biomass such as root biomass, shoot biomass, etc.;
reproductive organs; and
propagules such as seed.

### Marker Assisted Breeding

Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

### Use as Probes in (Gene Mapping)

Use of nucleic acids encoding the protein of interest for genetically and physically mapping the genes requires only a nucleic acid sequence of at least 15 nucleotides in length. These nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acids encoding the protein of interest. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid encoding the protein of interest in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculenturn, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes (or null control plants) are individuals missing the transgene by segregation. Further, control plants are grown under equal growing conditions to the growing conditions of the plants of the invention, i.e. in the vicinity of, and simultaneously with, the plants of the invention. A “control plant” as used herein refers not only to whole plants, but also to plant parts, including seeds and seed parts.

### DESCRIPTION OF FIGURES

The present invention will now be described with reference to the following figures in which:

FIG. 1 is an RNA gel blot analysis on the expressions of OsNAC1. (a) Expression of OsNAC1 in response to stress conditions in rice. 14-d-old seedlings were exposed to drought, high salinity, ABA, or low temperature for the indicated time points. For drought stress, the seedlings were air dried at 28° C.; for high-salinity stress, seedling were exposed to 400 mM NaCl at 28° C.; for low-temperature stress, seedling were exposed to 4° C.; for ABA treatment, seedlings were exposed to a solution containing 100 μM ABA. (b) RNA gel-blot analysis for three homozygous T₅ lines of RCc3:OsNAC1, GOS2:OsNAC1 and NT plants. Equal loading of RNAs were determined by using ethidium bromide (EtBr) staining. (−) and (+) represent null and transgenic lines, respectively.

FIG. 2 Stress tolerance of RCc3:OsNAC1 ad GOS2:OsNAC1 plants at the vegetative stage. (a) Images of plants during drought stress. Three independent homozygoues T₅ lines of RCc3:OsNAC1 and GOS2:OsNAC1 plants and NT controls were grown for two weeks, subjected to 5 d of drought stress and followed by 7 d of re-watering in the greenhouse indicated by plus (+) sign. (b) Comparison of the chlorophyll fluorescence (Fᵥ/Fₘ) of rice plants exposed to drought, high-salinity, and low-temperature stress conditions. Each data point represents the mean±SE of triplicate experiments (n=10).

FIG. 3 Agronomic traits of RCc3:OsNAC1 and GOS2:OsNAC1 plants in the field under normal (a) and drought (b) conditions for two cultivating seasons (2009-2010). Agronomic traits of three independent homozygous T₅ (2009) and T₆ (2010) lines for each transgenic plant together with NT controls were plotted using Microsoft Excel. Each data point represents the percentage of the mean values (n=30) with the NT plants assigned as 100%. CL, culm length; PL, panicle length; NP, number of panicles per hill; NSP, number of spikelets per panicle; TNS, total number of spikelets; FR, filling rate; NFG, number of filled grains; TGW, total grain weight; 1000 GW, 1,000 grain weight.

FIG. 4 Comparison of the root growth of RCc3:OsNAC1, GOS2:OsNAC1 and NT plants grown at the heading stage of reproduction. (a) Upper panel shows representative roots for each plant while lower panel shows 1 representative root for each plant. Bars=10 cm and 2 mm in upper and lower panels, respectively. (b) Light-microscopic images of cross-sectioned transgenic and NT plant roots. The whole-cross-section of the roots (top panel), vascular bundles within the stele (middle panel), and the epidermis and part of the cortex (bottom panel). co, cortex; xy, xylem; ae, aerenchyma; epidermis indicated by arrowhead. Bars=500 μm in top panel, 100 μm in middle and bottom panels. (c) The volume, length, dry weight and diameter of transgenic plant roots normalized to NT. Values are the means±SD of 50 roots (10 roots from each of five plants). Asterisks (**) indicate significant mean difference at the 0.01 level (LSD).

FIG. 5 shows RNA gel-blot analysis on the expressions of OsNAC5

A, Ten μg of total RNA was prepared from the leaf and root tissues of 14 d-old seedlings exposed to drought, high salinity, ABA or low temperature for the indicated time periods. For drought stress, the seedlings were air-dried at 28° C.; for high-salinity stress, seedlings were exposed to 400 mM NaCl at 28° C.; for low-temperature stress, seedlings were exposed to 4° C.; for ABA treatment, seedlings were exposed to a solution containing 100 μM ABA. Total RNAs were blotted and hybridized with OsNAC5 gene-specific probes. The blots were then reprobed for the Dip1 (Oh et al., 2005b) and rbcS (Jang et al., 1999) genes, which were used as markers for up- and down-regulation, respectively, of key genes following stress treatments. Ethidium bromide (EtBr) staining was used to determine equal loading of RNAs.
B, RNA gel-blot analyses were performed using total RNA preparations from the roots and leaves of three homozygous T₅ lines of RCc3:OsNAC5 and GOS2:OsNAC5 plants, respectively, and of non-transgenic (NT) control plants. The blots were hybridized with OsNAC5 gene specific probes, and also reprobed for rbcS and Tubulin. Ethidium bromide staining was used to determine equal loading of RNAs. (−) nullizygous (segregants without transgene) lines, (+) transgenic lines.
FIG. 6 shows stress tolerance of RCc3:OsNAC5 ad GOS2:OsNAC5 plants
A. The appearance of transgenic plants during drought stress. Three independent homozygous T₆ lines of RCc3:OsNAC5 and GOS2:OsNAC5 plants and non-transgenic (NT) controls were grown for 4 weeks, subjected to 3 days of drought stress and followed by 7 days re-watering in the greenhouse. Images were taken at the indicated time points. ‘+’ denotes the number of re-watering days under normal growth conditions.
B. Changes in the chlorophyll fluorescence (Fv/Fm) of rice plants under drought, high salinity and low temperature stress conditions. Three independent homozygous T₆ lines of RCc3:OsNAC5 and GOS2:OsNAC5 plants and NT controls grown in MS medium for 14 days were subjected to various stress conditions as described in the Examples section. After these stress treatments, the Fv/Fm values were measured using a pulse modulation fluorometer (mini-PAM, Walze, Germany). All plants were grown under continuous light of 150 μmol m⁻² s⁻¹ prior to stress treatments. Each data point represents the mean±SE of triplicate experiments (n=10).
C. The L-band of the plants under drought conditions was revealed through the difference kinetics at the F_{O} to F_{K} computed using the equation ΔW_{OK}=V_{OKsample}−V_{OKcontrol}; left axis. Double normalization at the O to K phase; Vₒₖ=(Fₜ−F_{O})/(F_{K}−F_{O}); right axis.
D. Events for V_{OI}≧1.0 illustrating the differences in the pool size of the end electron acceptors; V_{OI}=(Fₜ−F_{O})/(Fₜ−F_{O}) under normal and drought conditions.

FIG. 7 shows agronomic traits of RCc3:OsNAC5 and GOS2:OsNAC5 plants grown in the field under both normal (A) and drought (B) conditions

Spider plots of the agronomic traits of three independent homozygous T₅ and T₆ lines of RCc3:OsNAC5 and GOS2:OsNAC5 plants and corresponding non-transgenic (NT) controls under both normal and drought conditions were drawn using Microsoft Excel. Each data point represents the percentage of the mean values (n=30) listed in Table III and IV. The mean measurements from the NT controls were assigned a 100% reference value. CL, culm length; PL, panicle length; NP, number of panicles per hill; NSP, number of spikelets per panicle; TNS, total number of spikelets; FR, filling rate; NFG, number of filled grains; TGW, total grain weight; 1,000 GW, thousand grain weight.

FIG. 8 shows the difference in root growth of RCc3:OsNAC5 and GOS2:OsNAC5 plants

A, The root volume, length, dry weight and diameter of RCc3:OsNAC5 and GOS2:OsNAC5 plants are normalized to those of NT control roots. ** The mean difference is significant at the 0.01 level (LSD). Values are the means±SD of 50 roots (10 roots from each of 5 plants).
B, One representative root of RCc3:OsNAC5, GOS2:OsNAC5 and NT control plants that were grown to the heading stage of reproduction. Scale Bars=2 mm.
C, Light microscopic images of cross-sectioned RCc3:OsNAC5, GOS2:OsNAC5 and NT roots. The position of the metaxylem vessel (Me) and aerenchyma (Ae) are indicated. Scale bars, 500 μm in upper panels and 100 μm in middle and lower panels.

FIG. 9 represents a multiple alignment of various NAC1 polypeptides. The asterisks indicate identical amino acids among the various protein sequences, colons represent highly conserved amino acid substitutions, and the dots represent less conserved amino acid substitution; on other positions there is no sequence conservation. These alignments can be used for defining further motifs or signature sequences, when using conserved amino acids.

FIG. 10 represents a multiple alignment of various NAC5 polypeptides. The asterisks indicate identical amino acids among the various protein sequences, colons represent highly conserved amino acid substitutions, and the dots represent less conserved amino acid substitution; on other positions there is no sequence conservation. These alignments can be used for defining further motifs or signature sequences, when using conserved amino acids.

### EXAMPLES

The present invention will now be described with reference to the following examples, which are by way of illustration only. The following examples are not intended to limit the scope of the invention. Unless otherwise indicated, the present invention employs conventional techniques and methods in plant biology, molecular biology, bioinformatics and plant breedings.

For DNA manipulation, unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### A: Experimental Procedures

### (i) Plasmid Construction and Transformation of Rice with OsNAC1

The coding region of OsNAC1 was amplified using the primer pairs: forward (5′-ATGGGGATGGGGATGAGGAG-3′), reverse (5′-TCAGAACGGGACCATGCCCA-3′) from the total RNA using the RT-PCR system (Promega) according to the manufacturer's instructions. For overexpression in rice, the cDNA for OsNAC1 was linked to the GOS2 promoter for constitutive expression, and to the RCc3 promoter for root specific expression using the Gateway system (Invitrogen, Carlsbad, Calif.). Plasmids were introduced into Agrobacterium tumefaciens LBA4404 by triparental mating and embryogenic (Oryza sativa cv Nipponbare) calli from mature seeds were transformed as previously described (Jang et al., 1999).

### (ii) Plasmid Construction and Transformation of Rice with OsNAC5

The coding region of OsNAC5 (AK102475) was amplified from rice total RNA using an RT-PCR system (Promega, Wis.), according to the manufacturer's instructions. Primer pairs were as follows: forward (5′-ATGGAGTGCGGTGGTGCGCT-3′) and reverse (5′-TTAGAACGGCTTCTGCAGGT-3′). To enable the overexpression of the OsNAC5 gene in rice, the cDNA for this gene was linked to the GOS2 promoter for constitutive expression, and the RCc3 promoter for root specific expression using the Gateway system (Invitrogen, Carlsbad, Calif.). Plasmids were introduced into Agrobacterium tumefaciens LBA4404 by triparental mating and embryogenic (Oryza sativa cv Nipponbare) calli from mature seeds were transformed as previously described (Jang et al., 1999).

### (iii) Drought Treatments of Rice Plants at Vegetative Stage

Seeds from transgenic and non-transgenic (NT) rice (Oryza sativa cv Nipponbare) plants were germinated in half-strength MS solid medium and placed in a dark growth chamber at 28° C. for 4 days. Seedlings were transplanted into soil and then grown in a greenhouse (16-h-light/8-h-dark cycles) at 28-30° C. Before undertaking the drought-stress experiments, eighteen seedlings from each transgenic and non-transgenic lines were grown in pots (3×3×5 cm; 1 plant per pot) for four weeks. Drought stress was simulated by withholding water to the seedling for 3-5 days while recovery tests were performed by re-watering the drought-stressed plants and observed for 7 days. The numbers of plants that survived or continued to grow were then scored.

### (iv) RNA Gel-Blot Analysis NAC5

Rice (Oryza sativa cv Nipponbare) seeds were germinated in soil and grown in a glasshouse (16 h light/8 h dark cycle) at 28° C. For high-salinity and ABA treatments, 14-days-old seedlings were transferred to nutrient solution containing 400 mM NaCl or 100 μM ABA for the indicated periods in the glasshouse under continuous light of approximately 1000 μmol/m²/s. For drought treatment, 14-days-old seedlings were excised and air dried for the indicated time course under continuous light of approximately 1000 μmol/m²/s. For low-temperature treatments, 14-days-old seedlings were exposed at 4° C. in a cold chamber for the indicated time course under continuous light of 150 μmol/m²/s. The preparation of total RNA and RNA gel-blot analysis was performed as reported previously (Jang et al., 2002).

### (v) Northern Blot Analysis

Seeds from rice (Oryza sativa cv Nipponbare) were germinated in soil and grown in a glasshouse (16 h light/8 h dark cycle) at 22° C. The total RNA was prepared from 14-days-old seedlings exposed to drought, high salinity, ABA, or low temperature for the indicated time points. For high-salinity and ABA treatments, seedlings were transferred to nutrient solution containing 400 mM NaCl or 100 μM ABA for the indicated periods in the glasshouse under continuous light of approximately 1000 μmol/m²/s. For drought treatment, seedlings were excised and air dried for the indicated time course under continuous light of approximately 1000 μmol/m²/s. For low-temperature treatments, seedlings were exposed at 4° C. in a cold chamber for the indicated time course under continuous light of 150 μmol/m²/s. 10 μg of total RNAs were blotted and hybridized with OsNAC1 gene-specific probes. The blots were then reprobed with the Dip1 gene, which was used as a marker for the up-regulation of key genes following stress treatments. Ethidium bromide (EtBr) staining was used to determine equal loading of RNAs. Samples for the RNA gel-blot analysis was preared from the total RNA (10 μg) of leaf and root samples for each of the three homozygous T₅ lines of RCc3:OsNAC1, GOS2:OsNAC1 and NT plants. Blots were hybridized with OsNAC1 gene-specific probe and reprobed for RbcS and Tubulin. Equal loading of RNAs were determined by using ethidium bromide (EtBr) staining. The preparation of total RNA and RNA gel-blot analysis was followed to that Jang et al. (2002).

### (vi) Measurement of Chlorophyll Fluorescence Under Drought, High-Salinity and Low Temperature Conditions

Seeds from transgenic and non-transgenic rice (Oryza sativa cv Nipponbare) plants were germinated and grown in half-strength MS solid medium for 14 d. The growth chamber had the following light and dark settings 16-h-light of 150 μmol m⁻² s⁻¹/8-h-dark cycles at 28° C. The green portions of approximately 10 seedlings were then cut using a scissors prior to stress treatments in vitro. All stress conditions were conducted under continuous light at 150 μmol m⁻² s⁻¹. For low-temperature stress administration, the seedlings were incubated at 4° C. in water for up to 6 h. High-salinity stress was induced by incubation in 400 mM NaCl for 2 h at 28° C. To simulate drought stress, the plants were air-dried for 2 h at 28° C. Fᵥ/Fₘ values were then measured as previously described (Oh et al 2005).

### (vii) Rice 3′-Tiling Microarray Analysis

Rice 3′-Tiling Microarray was used for expression profiling analysis as previously described (Oh et al., 2009). Transgenic and non-transgenic rice (Oryza sativa cv Nipponbare) seeds were germinated in soil and grown in a glasshouse (16 h light/8 h dark cycle) at 22° C. To identify stress-inducible NAC genes in rice, total RNA (100 μg) was prepared from 14-d-old leaves of plants subjected to drought, high-salinity, ABA, and low-temperature stress conditions. For the high salinity and ABA treatments, the 14-d-old seedlings were transferred to a nutrient solution containing 400 mM NaCl or 100 μM ABA for 2 h in the greenhouse under continuous light of approximately 1000 μmol m⁻² s⁻¹. For drought treatment, 14-d-old seedlings were air-dried for 2 h also under continuous light of approximately 1000 μmol m⁻² s⁻¹. For low temperature treatments, 14-d-old seedlings were exposed at 4° C. in a cold chamber for 6 h under continuous light of 150 μmol m⁻² s⁻¹. For identification of genes up-regulated in RCc3:OsNAC1, GOS2:OsNAC1 plants, total RNA (100 μg) was prepared from root and leaf tissues of 14-d-old transgenic and non-transgenic rice seedlings (Oryza sativa cv Nipponbare) grown under normal growth conditions.

### (viii) Drought Treatments and Grain Yield Analysis of Rice Plants in the Field for Two (2009 and 2010) Years

To evaluate yield components of transgenic plants under normal field conditions, three independent T₅ (2009) and T₆ (2010) homozygous lines of the RCc3:OsNAC1 and GOS2:OsNAC1 plants, together with non-transgenic (NT) controls were transplanted to a paddy field at the Rural Development Administration, Suwon, Korea (2009) and Kyungpook National University, Gunwi, Korea (2010). A randomized design was employed with three replicates for the two cultivating seasons 2009-2010. Seedlings were randomly transplanted 25 d after sowing within a15×30 cm spacing with single seedling per hill. Fertilizer was applied at 70N/40P/70K kg ha⁻¹ after the last paddling and 45 d after transplantation. Yield parameters were scored for 30 plants per transgenic line per season. Plants located at borders were excluded from data scoring.

To evaluate yield components of transgenic plants under drought field conditions, three independent T₅ (2009) and T₆ (2010) homozygous lines of each of the RCc3:OsNAC1 and GOS2:OsNAC1 plants, and NT controls, were transplanted to a removable rain-off shelter (located at Myongji University, Yongin, Korea) with a 1 meter deep container filled with natural paddy soil.

The experimental design, transplanting spacing, use of fertilizer, drought treatments and scoring of agronomic traits was employed as described (Oh et al., 2009). When the plants grown under normal and drought conditions had reached maturity and the grains had ripened, they were harvested and threshed by hand (separation of seeds from the vegetative parts of the plant). The unfilled and filled grains were then taken apart, independently counted using a Countmate MC1000H (Prince Ltd, Korea), and weighed. The following agronomic traits were scored: panicle length (cm), number of panicles per hill, number of spikelets per panicle, number of spikelets per hill, filling rate (%), number of filled spikelets per hill, total grain weight (g), and 1,000 grain weight (g). The results from three independent lines were separately analyzed by one way ANOVA and compared with those of the NT controls. The ANOVA was used to reject the null hypothesis of equal means of transgenic lines and NT controls (p<0.05). SPSS version 16.0 was used to perform these statistical analyses.

The procedure above was also used for RCc3:OsNAC5 and GOS2:OsNAC5 plants.

### (ix) Microscopic Examination of Roots

Microscopic examination of roots was performed as described by Jeong et al. (2010). As an overview, the roots of transgenic and non-transgenic plants at the panicle heading stage were fixed with a modified Karnovsky's fixative at 4° C. overnight and washed with the same buffer three times for 10 min each. They were post-fixed in the same buffer at 4° C. for 2 h and washed with distilled water two times briefly. The post-fixed root tissues were enbloc stained at 4° C. overnight. They were dehydrated in a graded ethanol series (30, 50, 70, 80, 95, and 100%) and three times in 100% ethanol for 10 min each. Dehydrated samples were further treated with propylene oxide as a transitional fluid two times for 30 min each and embedded in Spurr's medium. Ultrathin sections (approximately 1 μm thick) were made with a diamond knife by an ultra-microtome (MT-X; RMC Inc., Tucson, Ariz.). The sections were stained with 1% toluidine blue and observed and photographed under a light microscope.

### (x) JIP Analysis

The chlorophyll a fluorescence transients of the plants were measured using the Handy-PEA fluorimeter (Plant Efficiency Analyzer, Hansatech Instruments Ltd., King's Lynn Norfolk, PE 30 4NE, UK), as described previously (Redillas et al., 2011a and 2011 b). Plants were dark-adapted for at least 30 min to ensure sufficient opening of reaction centers (RCs) i.e. the RCs are fully oxidized. Two plants were chosen for each of the three independent T₆ homozygous lines. The tallest and the visually healthy-looking leaves were selected for each plant and measured at their apex, middle and base parts. The readings were averaged using the Handy PEA Software (version 1.31). The Handy-PEA fluorimeter was set using the following program: the initial fluorescence was set as O (50 μs), J (2 ms) and I (30 ms) are intermediates, and P as the peak (500 ms-1s). The transients were induced by red light at 650 nm of 3,500 μmol photons m⁻²s⁻¹ provided by the 3 light-emitting diodes, focused on a spot of 5 mm in diameter and recorded for 1 s with 12 bit resolution. Data acquisition was set at every 10 μs (from 10 μs to 0.3 ms), every 0.1 ms (from 0.2 to 3 ms), every 1 ms (from 3 to 30 ms), every 10 ms (from 30 to 300 ms) and every 100 ms (from 300 ms to 1 s). Normalizations and computations were performed using the Biolyzer 4HP software (v4.0.30.03.02) according to the equations of the JIP-test. The difference kinetics computed for the OK phase (ΔW_{OK}) was performed by subtracting the normalized data of samples (V_{OKsample}) by the untreated NT (V_{OKcontrol}). Normalization for each data set performed following the equation V_{OK}=(Fₜ−F_{O})/(F_{K}−F_{O}). The graphs were made using OriginPro 8 SR0 v9.0724 (B724).

### B: Results

### Example 1

### Transgenic Overexpression of OsNAC1 Confers Stress Tolerance at the Vegetative Stage of Growth

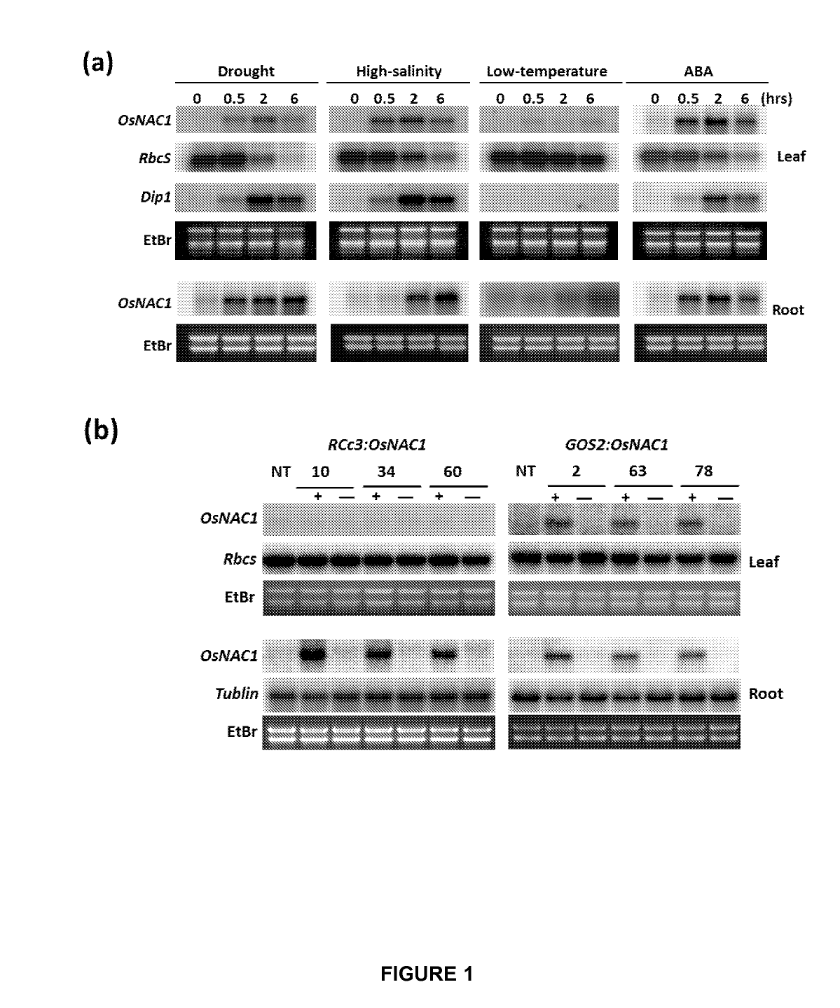

We performed RNA gel blot analysis using total RNAs from leaves and roots of 14-d-old seedlings exposed to drought, high-salinity, low temperature and ABA in a time course. Expression of endogenous OsNAC1 in rice leaves and roots was up-regulated significantly by drought, high-salinity and ABA but weakly by low-temperature conditions (FIG. 1a). To overexpress OsNAC1 in transgenic rice plants, the full-length cDNA of OsNAC1 was linked to two different promoters, RCc3 for root-specific expression (RCc3:OsNAC1) and GOS2 for constitutive expression (GOS2:OsNAC1). Fifteen to twenty independent transgenic lines per construct were produced through the Agrobacterium-mediated transformation method. T₁₋₆ seeds from transgenic lines that grew normal without stunting were collected and three independent T₅₋₆ homozygous lines of both RCc3:OsNAC1 and GOS2:OsNAC1 plants were selected for further analysis. The expression of RCc3:OsNAC1 and GOS2:OsNAC1 was confirmed by RNA gel-blot analysis in both roots and leaves (FIG. 1b). Expression of the transgene OsNAC1 was not detected in the leaves of RCc3:OsNAC1 plants while the roots showed high levels of transgene expression validating the root-specificity of the RCc3 promoter. Expression levels of the transgene were similarly increased in both roots and leaves of GOS2:OsNAC1 plants. In addition, expression levels of the transgene were higher in roots of RCc3:OsNAC1 plants than in roots of GOS2:OsNAC1 plants while those of the reference Tublin remained consistent.

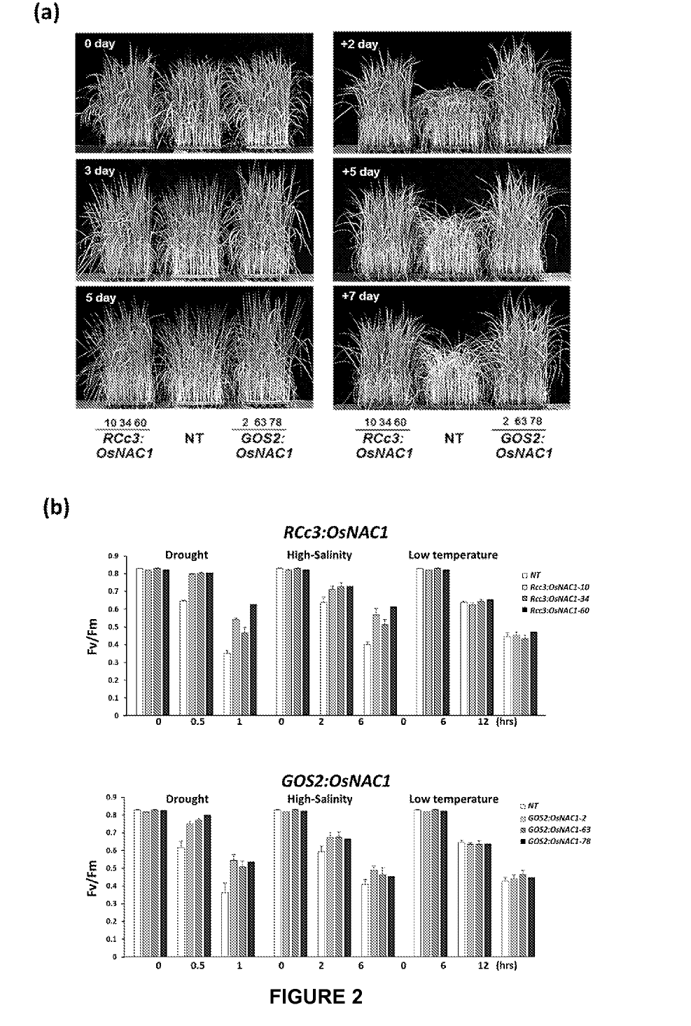

To evaluate stress-tolerance of OsNAC1 overexpressors at the vegetative stage of growth, four-week-old transgenic and non-transgenic (NT) control plants were subjected to drought stress for up to 5 d (FIG. 2a). Transgenic plants showed delayed leaf rolling compared to NT during drought treatments. After re-watering, transgenic plants started to recuperate while NT plants continuously withered with no signs of recovery, demonstrating drought tolerance of the transgenic plants at the vegetative stage. Since environmental stresses affect the photosynthetic machinery of plants, the maximum photochemical efficiency of PSII (Fᵥ/Fₘ: Fᵥ, variable fluorescence; Fₘ, maximum fluorescence) was measured using a pulse amplitude modulation fluorometer (FIG. 2b). Fourteen-d-old plants were subjected to a time course of drought, high-salinity and low-temperature stress and their Fᵥ/Fₘ, values determined. Both under drought and high-salinity conditions, RCc3:OsNAC1 and GOS2:OsNAC1 plants showed higher Fᵥ/Fₘ, values than NT control plants by 10-30% depending on the extent of stress and transgenic lines. Under low temperature conditions, in contrast, no difference in Fᵥ/Fₘ, values was observed between the transgenic and NT control plants. Together, these results indicate enhanced tolerance of both transgenic plants to drought stress at the vegetative stage of growth.

Table I below shows: Analysis of seed production parameters in RCc3:OsNAC1 and GOS2:OsNAC1 plants under normal growth conditions for 2009 and 2010.

TABLE I
Analysis of seed production parameters in RCc3: OsNAC1 and GOS2:
OsNAC1 plants under normal growth conditions for 2009 and 2010.

| | Construct | | | | | | | | | |
|---|---|---|---|---|---|---|---|---|---|---|
| | Culm | | Panicle | | No. of | | No. of | | No. of Total | |
| | Length | | Length | | Panicles | | Spikelets | | Spikelets | |
| | (cm) | | (cm) | | (/hill) | | (/panicle) | | (/hill) | |
| Normal | 2009 | 2010 | 2009 | 2010 | 2009 | 2010 | 2009 | 2010 | 2009 | 2010 |
| NT (Nipponbare) | 70.92 | 89.50 | 19.13 | 21.03 | 9.70 | 13.77 | 90.55 | 107.65 | 944.73 | 1468.23 |
| RCc3: OsNAC1-10 | 74.57 * | 90.53 | 20.12 * | 21.87 * | 10.87 * | 13.80 | 91.18 | 111.45 | 976.97 | 1545.63 |
| % Δ | 5.15 | 1.15 | 5.14 | 3.99 | 12.03 | 0.22 | 0.69 | 3.53 | 3.41 | 5.27 |
| RCc3: OsNAC1-34 | 74.85 * | 90.53 | 20.20 * | 22.13 * | 10.57 | 14.27 | 91.44 | 113.39 | 994.07 | 1604.30 |
| % Δ | 5.55 | 1.15 | 5.57 | 5.23 | 8.93 | 3.63 | 0.98 | 5.33 | 5.22 | 9.27 |
| RCc3: OsNAC1-60 | 69.75 | 85.70 * | 20.60 * | 22.57 * | 10.20 | 13.80 | 97.64 * | 130.10 * | 1012.60 | 1771.87 * |
| % Δ | −1.65 | −4.25 | 7.67 | 7.32 | 5.15 | 0.22 | 7.83 | 20.85 | 7.18 | 20.68 |
| NT (Nipponbare) | 70.92 | 89.50 | 19.13 | 21.03 | 9.70 | 13.77 | 90.55 | 107.65 | 944.73 | 1468.23 |
| GOS2: OsNAC1-2 | 75.83 * | 89.70 | 20.97 * | 22.83 * | 10.80 * | 14.63 | 97.31 * | 121.61 * | 1066.73 * | 1749.50 * |
| % Δ | 6.93 | 0.22 | 9.58 | 8.56 | 11.34 | 6.25 | 7.47 | 12.97 | 12.91 | 19.16 |
| GOS2: OsNAC1-63 | 72.80 * | 86.80 * | 20.33 * | 22.10 * | 12.30 * | 13.97 | 91.55 | 115.87 | 1113.20 * | 1566.37 |
| % Δ | 2.66 | −3.02 | 6.27 | 5.09 | 26.80 | 1.45 | 1.10 | 7.64 | 17.83 | 6.68 |
| GOS2: OsNAC1-78 | 76.92 * | 90.93 | 21.80 * | 23.83 * | 11.47 * | 14.27 | 103.26 * | 121.81 * | 1175.63 * | 1665.07 * |
| % Δ | 8.46 | 1.60 | 13.94 | 13.31 | 18.21 | 3.63 | 14.04 | 13.15 | 24.44 | 13.41 |

| | Construct | | | | | | | |
|---|---|---|---|---|---|---|---|---|
| | Filling | | No. of Filled | | Total Grain | | 1000 Grain | |
| | Rate | | Spikelets | | Weight | | Weight | |
| | (%) | | (/hill) | | (g) | | (g) | |
| Normal | 2009 | 2010 | 2009 | 2010 | 2009 | 2010 | 2009 | 2010 |
| NT (Nipponbare) | 92.08 | 82.74 | 880.50 | 1215.23 | 20.76 | 27.82 | 24.28 | 22.92 |
| RCc3: OsNAC1-10 | 92.94 | 85.41 * | 908.07 | 1318.00 | 23.47 * | 31.64 * | 25.84 * | 24.09 * |
| % Δ | 0.93 | 3.23 | 3.13 | 8.46 | 13.04 | 13.73 | 6.43 | 5.10 |
| RCc3: OsNAC1-34 | 92.32 | 85.87 * | 912.27 | 1372.40 | 24.01 * | 32.36 * | 25.47 * | 23.92 |
| % Δ | 0.26 | 3.78 | 3.61 | 12.93 | 15.64 | 16.32 | 4.89 | 4.36 |
| RCc3: OsNAC1-60 | 90.38 * | 82.15 | 928.80 | 1458.50 * | 24.54 * | 31.88 * | 23.70 | 21.87 |
| % Δ | −1.84 | −0.71 | 5.49 | 20.02 | 18.17 | 14.59 | −2.38 | −4.58 |
| NT (Nipponbare) | 92.08 | 82.74 | 880.50 | 1215.23 | 20.76 | 27.82 | 24.28 | 22.92 |
| GOS2: OsNAC1-2 | 91.10 | 82.27 | 945.33 | 1437.10 * | 24.93 * | 33.11 * | 26.38 * | 23.13 |
| % Δ | −1.06 | −0.57 | 7.36 | 18.26 | 20.07 | 19.02 | 8.66 | 0.92 |
| GOS2: OsNAC1-63 | 87.22 * | 81.53 | 971.57 * | 1279.80 | 26.35 * | 31.64 * | 27.13 * | 24.86 * |
| % Δ | −5.28 | −1.46 | 10.34 | 5.31 | 26.91 | 13.73 | 11.73 | 8.46 |
| GOS2: OsNAC1-78 | 90.78 | 81.94 | 1068.97 * | 1365.50 | 27.45 * | 32.84 * | 25.70 * | 24.08 * |
| % Δ | −1.41 | −0.97 | 21.40 | 12.37 | 32.22 | 18.04 | 5.85 | 5.06 |

Each parameter value represents the mean ± SD (n = 30) for RCc3: OsNAC1 and GOS2: OsNAC1 plants and the respective NT controls.
Percentage differences (% Δ) between the values for the RCc3: OsNAC1 and GOS2: OsNAC1 plants and for the respective NT controls are presented.
Asterisk (*) indicate significant difference (p < 0.05).

### Example 2

### Overexpression of OsNAC1 Increases Grain Yield Under Both Normal and Drought Conditions

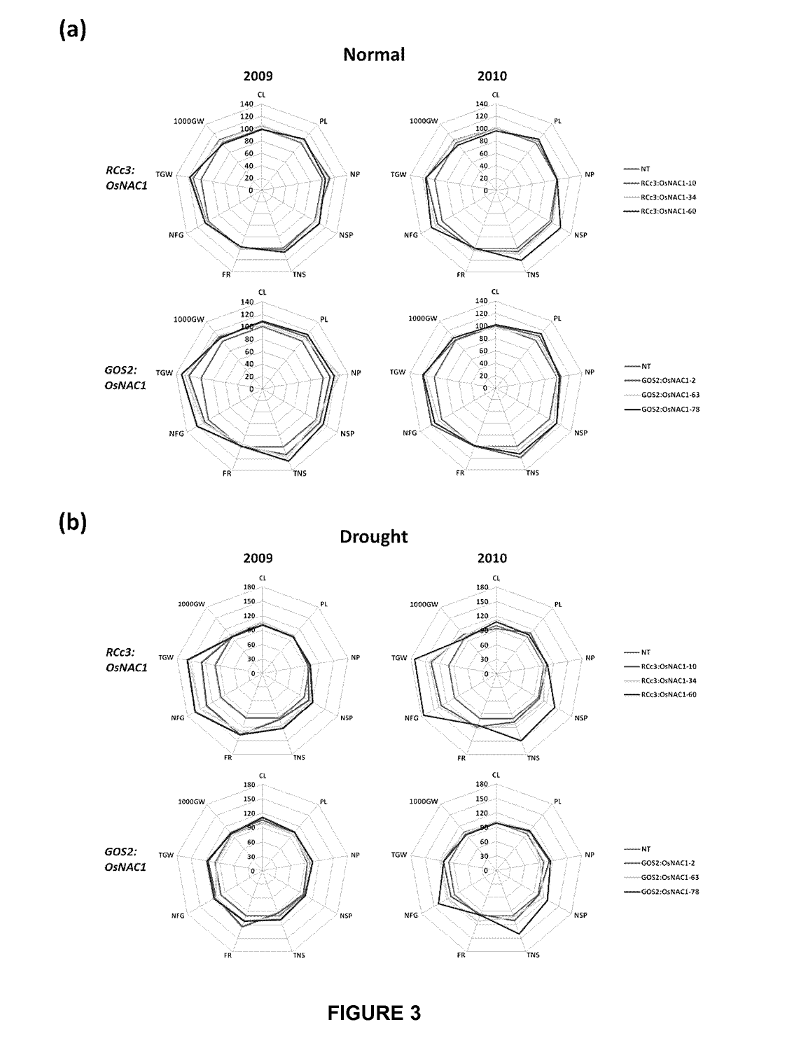

Yield components of the transgenic plants under normal and field drought conditions were evaluated for two cultivating seasons (2009 and 2010). Three independent T₅ (2009) and T₆ (2010) homozygous lines of RCc3:OsNAC1 and GOS2:OsNAC1 plants, together with non-transgenic (NT) controls, were transplanted to a paddy field and grown to maturity. Yield parameters were scored for 30 plants per transgenic line from three replicates. Data sets from two years of field test were generally consistent and total grain weights of the RCc3:OsNAC1 and the GOS2:OsNAC1 plants were increased by 13-18% and 13-32%, respectively. The increase of total grain weight was due mainly to the increased panicle length in RCc3:OsNAC1 plants and to the increased panicle length and number in GOS2:OsNAC1 plants (FIG. 3a; Table I).

To test the transgenic plants under drought conditions, three independent T₅ and T₆ lines of RCc3:OsNAC1 and GOS2:OsNAC1 plants were transplanted to a paddy field with a removable rain-off shelter. Plants were exposed to drought stress at the panicle heading stage (from 10 d before heading and 10 d after heading). The level of drought stress imposed under the rain-off shelter was equivalent to those that give 40-50% of total grain weight obtained under normal growth conditions, which was evidenced by the difference in levels of total grain weight of NT plants between the normal and drought conditions (Supplementary Tables S1 and S2). Statistical analysis of the yield parameters scored for two cultivating seasons showed that the decrease in grain yield under drought conditions was significantly smaller in the RCc3:OsNAC1 plants than that observed in the NT controls. Specifically, in the drought-treated RCc3:OsNAC1 plants, the filling rate was 18-36% higher than the drought-treated NT plants, which resulted in the increase in total grain weight by 28-72%, depending on transgenic line (FIG. 3b; Table II). In the drought-treated GOS2:OsNAC1 plants, in contrast, the total grain weight remained similar to the drought-treated NT controls. Given similar levels of drought tolerance during the vegetative stage in the RCc3:OsNAC1 and GOS2:OsNAC1 plants, the differences in total grain weight under field drought conditions were unexpected.

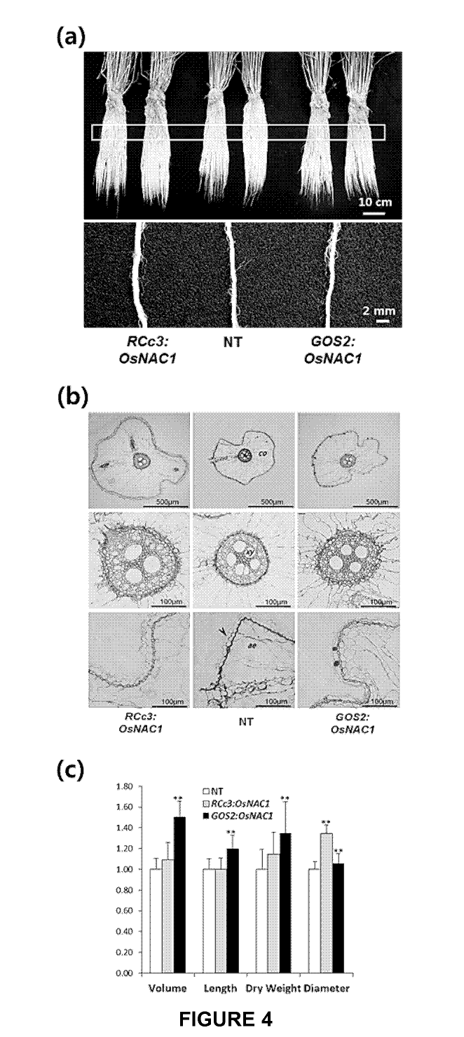

The root architecture of transgenic plants was also observed, measuring root volume, length, dry weight and diameter of RCc3:OsNAC1, GOS2:OsNAC1 and NT plants grown to the heading stage of reproduction. As shown in FIG. 4b, root diameter of the RCc3:OsNAC1 and GOS2:OsNAC1 plants was thicker by 30% and 7% than that of NT control plants, respectively. Microscopic analysis of cross-sectioned roots revealed that the increase in root diameter was due to the enlarged stele, cortex and epidermis of RCc3:OsNAC1 roots. In particular, the aerenchyma (ae in FIG. 4b) was bigger in the RCc3:OsNAC1 roots compared to the GOS2:OsNAC1 and NT plants, which may have contributed to the enlargement of the RCc3:OsNAC1 roots along with enlarged stele. The fact that root-specific overexpression of OsNAC1 increases root diameter with larger aerenchyma was correlated with the enhanced drought tolerance of transgenic plants at the reproductive stage. The volume, length and dry weight of the GOS2:OsNAC1 roots increased by 50%, 20% and 35% relative to NT roots, respectively, suggesting that these parameters also affected the increase in grain yield of the plants under normal growth conditions.

Under normal growth conditions, both plants showed higher grain yield compared to non-transgenic (NT) controls. The improvement in the total grain weight of RCc3:OsNAC1 plants was due mainly to the increase in the panicle length whereas those of GOS2:OsNAC1 plants was due to a number of traits including panicle length, number of panicles, and number of spikelets. Under drought conditions, in contrast, RCc3:OsNAC1 plants significantly enhanced the total grain weight by 28-72% due mainly to the increase in filling rate while GOS2:OsNAC1 plants showed no significant changes in either trait.

The root-specific overexpression of OsNAC1 clearly played an important role in the improvement of rice yield particularly under drought conditions. The RCc3:OsNAC1 and GOS2:OsNAC1 plants at T₅ or later generations did not show any unwanted pleiotropic effects such as growth retardation, abnormal leaf shape and color, and panicle underdevelopment which were, if any, segregated out during the pre-screening at earlier generations. Thus in comparison to NT controls, the changes in responses exhibited by RCc3:OsNAC1 and GOS2:OsNAC1 plants at T₅₋₆ were contributed solely by the transgene. The root characteristics of RCc3:OsNAC1 plants at heading stage of reproduction showed an increase in root diameter as compared to those of NT controls and GOS2:OsNAC1 plants. The increase was apparently due to the enlarged xylem, bigger cortical cells and epidermis. The thick roots with enlarged xylem contribute to a better water flux and have lesser risk of cavitation than thin roots (Yambao et al., 1992). Also, bigger roots have a direct role in drought tolerance since the large size of root diameter is related to penetration (Clark et al., 2008; Nguyen et al., 1997) and branching (Fitter, 1991; Ingram et al., 1994) ability.

Table II below shows: Analysis of seed production parameters in RCc3:OsNAC1 and GOS2:OsNAC1 plants under drought stress conditions for 2009 and 2010.

TABLE II
Analysis of seed production parameters in RCc3: OsNAC1 and GOS2:
OsNAC1 plants under drought stress conditions for 2009 and 2010.

| | Construct | | | | | | | | | |
|---|---|---|---|---|---|---|---|---|---|---|
| | Culm | | Panicle | | No. of | | No. of | | No. of Total | |
| | Length | | Length | | Panicles | | Spikelets | | Spikelets | |
| | (cm) | | (cm) | | (/hill) | | (/panicle) | | (/hill) | |
| Drought | 2009 | 2010 | 2009 | 2010 | 2009 | 2010 | 2009 | 2010 | 2009 | 2010 |
| NT (Nipponbare) | 63.93 | 56.47 | 19.04 | 18.58 | 11.24 | 12.06 | 79.25 | 90.05 | 868.21 | 1089.50 |
| RCc3: OsNAC1-10(+) | 68.29 * | 53.14 | 19.25 | 20.42 * | 10.83 | 12.83 | 88.59 | 92.74 | 893.71 | 1174.78 |
| % Δ | 6.81 | −5.90 | 1.09 | 9.87 | −3.60 | 6.45 | 11.79 | 2.99 | 2.94 | 7.83 |
| RCc3: OsNAC1-34(+) | 68.50 * | 6.67 * | 18.96 | 19.56 | 11.42 | 12.28 | 91.35 | 102.00 | 1015.42 * | 1222.11 |
| % Δ | 7.14 | 12.74 | −0.44 | 5.23 | 1.59 | 1.84 | 15.27 | 13.28 | 16.96 | 12.17 |
| RCc3: OsNAC1-60(+) | 64.42 | 60.94 | 18.79 | 19.60 * | 11.33 | 13.00 * | 95.95 * | 125.96 * | 1064.08 * | 1623.04 * |
| % Δ | 0.75 | 7.91 | −1.31 | 5.49 | 0.85 | 7.83 | 21.08 | 39.89 | 22.56 | 48.97 |
| NT (Nipponbare) | 63.93 | 56.47 | 19.04 | 18.58 | 11.24 | 12.06 | 79.25 | 90.05 | 868.21 | 1089.50 |
| GOS2: OsNAC1-2(+) | 67.63 * | 56.06 | 19.31 | 19.61 | 10.78 | 13.50 | 78.12 | 91.80 | 812.67 | 1214.83 |
| % Δ | 5.77 | −0.74 | 1.42 | 5.53 | −4.05 | 11.98 | −1.42 | 1.94 | −6.40 | 11.50 |
| GOS2: OsNAC1-63(+) | 62.79 | 57.58 | 19.15 | 20.17 * | 11.04 | 11.22 | 82.42 | 101.90 | 896.21 | 1125.83 |
| % Δ | −1.79 | 1.97 | 0.55 | 8.52 | −1.75 | −6.91 | 4.00 | 13.16 | 3.23 | 3.33 |
| GOS2: OsNAC1-78(+) | 71.04 * | 55.53 | 19.90 * | 20.14 * | 11.91 | 13.83 * | 81.89 | 111.24 * | 942.96 | 1525.89 * |
| % Δ | 11.12 | −1.67 | 4.49 | 8.37 | 6.01 | 14.75 | 3.33 | 23.54 | 8.61 | 40.05 |

| | Construct | | | | | | | |
|---|---|---|---|---|---|---|---|---|
| | Filling | | No. of Filled | | Total Grain | | 1000 Grain | |
| | Rate | | Spikelets | | Weight | | Weight | |
| | (%) | | (/hill) | | (g) | | (g) | |
| Drought | 2009 | 2010 | 2009 | 2010 | 2009 | 2010 | 2009 | 2010 |
| NT (Nipponbare) | 47.36 | 47.62 | 419.74 | 515.33 | 8.58 | 10.09 | 20.21 | 19.49 |
| RCc3: OsNAC1-10(+) | 64.62 * | 56.44 * | 562.46 * | 679.22 | 11.05 * | 13.87 * | 20.00 | 20.79 |
| % Δ | 36.46 | 18.54 | 34.00 | 31.80 | 28.73 | 37.50 | −1.02 | 6.68 |
| RCc3: OsNAC1-34(+) | 61.63 * | 58.33 * | 626.88 * | 713.72 | 13.28 * | 14.35 * | 20.94 | 20.13 |
| % Δ | 30.15 | 22.50 | 49.35 | 38.50 | 54.70 | 42.24 | 3.65 | 3.30 |
| RCc3: OsNAC1-60(+) | 64.65 * | 54.34 | 680.04 * | 896.04 | 13.65 * | 17.37 * | 19.97 | 19.01 |
| % Δ | 36.51 | 14.12 | 62.02 | 73.88 | 59.03 | 72.14 | −1.16 | −2.45 |
| NT (Nipponbare) | 47.36 | 47.62 | 419.74 | 515.33 | 8.58 | 10.09 | 20.21 | 19.49 |
| GOS2: OsNAC1-2(+) | 58.88 * | 45.98 | 471.74 | 556.39 | 9.85 | 11.23 | 20.86 | 20.18 |
| % Δ | 24.34 | −3.43 | 12.39 | 7.97 | 14.81 | 11.23 | 3.22 | 3.57 |
| GOS2: OsNAC1-63(+) | 53.02 | 53.76 | 467.74 | 601.72 | 8.30 | 11.80 | 19.60 | 19.79 |
| % Δ | 11.95 | 12.90 | 11.44 | 16.76 | −3.28 | 16.96 | −3.01 | 1.54 |
| GOS2: OsNAC1-78(+) | 52.68 | 46.80 | 488.27 * | 712.28 | 10.06 | 11.15 | 20.60 | 18.88 |
| % Δ | 11.24 | −1.71 | 16.33 | 38.22 | 17.29 | 10.50 | 1.93 | −3.12 |

Each parameter value represents the mean ± SD (n = 30) for RCc3: OsNAC1 and GOS2: OsNAC1 plants and the respective NT controls.
Percentage differences (% Δ) between the values for the RCc3: OsNAC1 and GOS2: OsNAC1 plants and for the respective NT controls are presented.
Asterisk (*) indicate significant difference (p < 0.05).

### Example 3

### Identification of Genes Up-Regulated by Overexpressed OsNAC1

Expression profiling was performed for RCc3:OsNAC1 and GOS2:OsNAC1 roots to identify up-regulated genes following the overexpression of OsNAC1. Rice 3′-Tiling Microarray was performed on RNA samples extracted from the roots of 14-d-old plants grown under normal conditions. Each data set was obtained from duplicate biological samples. Statistical analysis using one-way ANOVA (p<0.01) identified 46 genes to be up-regulated more than 3-fold in RCc3:OsNAC1 and GOS2:OsNAC1 roots following OsNAC1 overexpression (Table I). Also identified were 9 and 28 genes that were specific to RCc3:OsNAC1 and to GOS2:OsNAC1 roots, respectively (Table A). The highly up-regulated target genes common to both transgenic roots include 9-cis-epoxycarotenoid dioxygenase, a gene for ABA biosynthesis, calcium-transporting ATPase, a component for Ca²⁺ signaling for cortical cell death (apoptosis) leading to aerenchyma formation, cinnamoyl CoA reductase 1, a gene involved in lignin biosynthesis for barrier formation (Casparian Strip) surrounding the aerenchyma. Interestingly, O-methyltransferase, a gene for suberin biosynthesis that is also necessary for barrier formation, was specifically up-regulated only in RCc3:OsNAC1 roots. Such target genes up-regulated specifically in the transgenic roots may account for the difference in root architecture, hence drought tolerance at the reproduction stage.

The common target genes include 9-cis-epoxycarotenoid dioxygenase, calcium-transporting ATPase and cinnamoyl CoA reductase 1. The oxidative cleavage of cis-epoxycarotenoids by 9-cis-epoxycarotenoid dehydrogenase (NCED) to generate xanthoxin is the critical and the rate-limiting step in the regulation of ABA biosynthesis (Tan et al., 1997). The NCED gene was up-regulated by more than 20-fold in both transgenic plants which may have contributed to the sensitivity of the plants when exposed to drought stress. The Ca²⁺-transporting-ATPase (Ca²⁺-ATPase) was up-regulated by 26- and 32-fold in RCc3:OsNAC1 and GOS2:OsNAC1 plants, respectively. A transient increase in cytosolic Ca²⁺, derived from either influx from the apoplastic space or released from internal stores, serves as an early response to low temperature, drought and salinity stress in plant cells (Knight, 2000). Coupled with the increase of cytosolic Ca²⁺ is the rupture of tonoplasts which also indicate early events preceding the death of root cortical cells followed by the formation of aerenchyma—the gas filled spaces in the cortical region of roots. This explains the contribution of bigger cortical cells observed in RCc3:OsNAC1 roots. Aerenchyma serves as anatomical adaptions in rice that help minimize loss of O₂ to the surrounding soil for respiration by the apical meristem. These structures include a suberized hypodermis and a layer of lignified cells immediately interior to the hypodermis, both of which are only slightly gas permeable (Drew et al., 2000). Interestingly, cinnamoyl-CoA reductase (CCR), a gene encoding a key enzyme (EC 1.2.144) in lignin biosynthesis, was up-regulated in RCc3:OsNAC1 and GOS2:OsNAC1 plants following OsNAC1 overexpression. CCR is the first enzyme specific to the biosynthetic pathway leading to production of monolignols p-coumaryl, coniferyl, and sinapyl alcohols, controlling the quantity and quality of lignin (Jones et al., 2001). Down-regulation of the AtCCR1, an Arabidopsis homologue, caused drastic alterations in the plant's phenotypes (Goujon et al., 2003). Also, the loss-of-function mutation in maize (Zmccr1^{-/-}) resulted in a slight decrease of lignin content and caused significant changes in lignin structure (Tamasloukht et al., 2011). The maize gene ZmCCR2 was found to be induced by drought conditions and can be detected mainly in roots (Fan et al., 2006). Along with CCR, cinnamyl alcohol dehydrogenase (CAD), another gene encoding an enzyme involved in lignin biosynthesis, was also up-regulated in both plants. CAD catalyzes the final conversion of hydroxycinnamoyl aldehydes (monolignals) to monolignols in lignin biosynthesis pathway (Sattler et al. 2009). Furthermore, O-methyltransferase, a gene encoding an enzyme (EC=2.1.1-) involved in suberin biosynthesis, was specifically up-regulated in RCc3:OsNAC1 plants. In Arabidopsis the mRNA ZRP4, which codes for O-methyltransferase, was found to accumulate preferentially in roots and is located predominantly in the region of the endodermis with low levels seen in the leaves, stems and other shoot organs (Held et al., 1993). The up-regulation of three O-methyltransferase genes using the root-specific promoter may have contributed to the enhanced drought tolerance of RCc3:OsNAC1 plants over GOS2:OsNAC1 and NT plants due to its involvement in suberin biosynthesis. Lignin, together with suberin, have major roles in impeding radial oxygen loss through lignification and/or suberization of the walls of root peripheral layers in a process called barrier formation. This barrier formation on the radial and transverse walls of endo- and exodermal cells is generally associated with Casparian Strips (CSs). The main function of CSs is to inhibit water and salt transport into the stele by blocking selective apoplastic bypass in the root (Ma et al, 2003). Cai et al. (2011) reported that the development of CSs on the endodermis and exodermis in the salt- and drought-tolerant Liaohan 109 occurred earlier than the moderately salt-sensitive Tianfeng 202 and the salt-sensitive Nipponbare. The group also reported that even without the salt in nutrient solution, the development of CSs in Liaohan 109 had been brought forward and increased. Thus, the results of microarray provided us insights on how the plants endured drought stress and how the regulation of genes was affected by the overexpression of OsNAC1 either specifically in roots or throughout the whole plant body.

Results from microarray showed 46 up-regulated target genes common to RCc3:OsNAC1 and GOS2:OsNAC1 roots (Table A). In addition, 9 and 28 target genes were found to be specifically up-regulated in RCc3:OsNAC1 and GOS2:OsNAC1 roots, respectively (Table A). The common target genes include 9-cis-epoxycarotenoid dioxygenase, calcium-transporting ATPase and cinnamoyl CoA reductase 1. The oxidative cleavage of cis-epoxycarotenoids by 9-cis-epoxycarotenoid dehydrogenase (NCED) to generate xanthoxin is the critical and the rate-limiting step in the regulation of ABA biosynthesis (Tan et al., 1997). The NCED gene was up-regulated for more than 20-fold in both transgenic plants which may have contributed to the sensitivity of the plants when exposed to drought stress. The Ca²⁺-transporting-ATPase (Ca²⁺-ATPase) was up-regulated by 26- and 32-fold in RCc3:OsNAC1 and GOS2:OsNAC1 plants, respectively. A transient increase in cytosolic Ca²⁺, derived from either influx from the apoplastic space or released from internal stores, serves as an early response to low temperature, drought and salinity stress in plant cells (Knight, 2000). Coupled with the increase of cytosolic Ca²⁺ is the rupture of tonoplasts which also indicate early events preceding the death of root cortical cells followed by the formation of aerenchyma—the gas filled spaces in the cortical region of roots. This explains the contribution of bigger cortical cells observed in RCc3:OsNAC1 roots. Aerenchyma serves as anatomical adaptions in rice that help minimize loss of O₂ to the surrounding soil for respiration by the apical meristem. These structures include a suberized hypodermis and a layer of lignified cells immediately interior to the hypodermis, both of which are only slightly gas permeable (Drew et al., 2000). Interestingly, cinnamoyl-CoA reductase (CCR), a gene encoding a key enzyme (EC 1.2.144) in lignin biosynthesis, was up-regulated in RCc3:OsNAC1 and GOS2:OsNAC1 plants following OsNAC1 overexpression. CCR is the first enzyme specific to the biosynthetic pathway leading to production of monolignols p-coumaryl, coniferyl, and sinapyl alcohols, controlling the quantity and quality of lignin (Jones et al., 2001). Down-regulation of the AtCCR1, an Arabidopsis homologue, caused drastic alterations in the plant's phenotypes (Goujon et al., 2003). Also, the loss-of-function mutation in maize (Zmccr1^{-/-}) resulted in a slight decrease of lignin content and caused significant changes in lignin structure (Tamasloukht et al., 2011). The maize gene ZmCCR2 was found to be induced by drought conditions and can be detected mainly in roots (Fan et al., 2006). Along with CCR, cinnamyl alcohol dehydrogenase (CAD), another gene encoding an enzyme involved in lignin biosynthesis, was also up-regulated in both plants. CAD catalyzes the final conversion of hydroxycinnamoyl aldehydes (monolignals) to monolignols in lignin biosynthesis pathway (Sattler et al. 2009). Furthermore, O-methyltransferase, a gene encoding an enzyme (EC=2.1.1-) involved in suberin biosynthesis, was specifically up-regulated in RCc3:OsNAC1 plants. In Arabidopsis the mRNA ZRP4, which codes for O-methyltransferase, was found to accumulate preferentially in roots and is located predominantly in the region of the endodermis with low levels seen in the leaves, stems and other shoot organs (Held et al., 1993). The up-regulation of three O-methyltransferase genes using the root-specific promoter may have contributed to the enhanced drought tolerance of RCc3:OsNAC1 plants over GOS2:OsNAC1 and NT plants due to its involvement in suberin biosynthesis as described above. Thus, the results of microarray provided us insights on how the plants endured drought stress and how the regulation of genes was affected by the overexpression of OsNAC1 either specifically in roots or throughout the whole plant body.

TABLE A
Up-regulated root-expressed genes in RCc3: OsNAC1 and GOS2:
OsNAC1 plants in comparison to non-transgenic controls.

| | Loc No^{a} | RCc3: OsNAC1 | | GOS2: OsNAC1 | |
|---|---|---|---|---|---|
| Gene Name | (IRGSP) | Mean^{b} | p-val^{c} | Mean^{b} | p-val^{c} |

Up-regulated genes in RCc3: OsNAC1 and GOS2: OsNAC1

| | | | | | |
|---|---|---|---|---|---|
| Protein kinase | Os01g0117600 | 3.60 | 0.00 | 3.04 | 0.00 |
| ABC transporter | Os01g0609300 | 3.39 | 0.00 | 4.44 | 0.00 |
| Peptidase aspartic | Os01g0937500 | 3.40 | 0.00 | 3.37 | 0.00 |
| Cytochrome P450 | Os02g0601400 | 5.35 | 0.00 | 2.91 | 0.00 |
| WAK3 | Os02g0807900 | 5.02 | 0.00 | 4.47 | 0.00 |
| Cinnamoyl CoA Reductase 1 | Os02g0811800 | 10.65 | 0.00 | 7.47 | 0.00 |
| Acyl-activating enzyme | Os03g0130100 | 3.09 | 0.00 | 3.31 | 0.00 |
| Phytosulfokine | Os03g0232400 | 3.69 | 0.00 | 2.85 | 0.00 |
| U-box | Os03g0240600 | 6.16 | 0.00 | 7.27 | 0.00 |
| Aspartyl protease | Os03g0318400 | 3.59 | 0.00 | 3.61 | 0.00 |
| High affinity K+ transporter 5 | Os03g0575200 | 5.28 | 0.00 | 5.03 | 0.00 |
| Copalyl diphosphate synthetase | Os04g0178300 | 3.50 | 0.00 | 3.56 | 0.00 |
| RLP (receptor-like protein kinase) | Os04g0202700 | 4.10 | 0.00 | 4.16 | 0.00 |
| MAPKKK9 | Os04g0339800 | 7.17 | 0.00 | 6.91 | 0.00 |
| WAK2 | Os04g0365100 | 4.60 | 0.00 | 3.56 | 0.00 |
| WAK2 | Os04g0368800 | 3.87 | 0.00 | 3.56 | 0.00 |
| Glutamate dehydrogenase | Os04g0543900 | 3.13 | 0.00 | 3.00 | 0.00 |
| Downy mildew resistnant 6 | Os04g0581000 | 4.49 | 0.00 | 4.55 | 0.00 |
| Oxidoreductase, 2OG-Fe(II) oxygenase | Os04g0581100 | 70.90 | 0.00 | 61.65 | 0.00 |
| Pyruvate kinase | Os04g0677300 | 3.27 | 0.00 | 4.05 | 0.00 |
| Zinc finger | Os05g0404700 | 3.11 | 0.00 | 5.46 | 0.00 |
| Aldo/keto reductase | Os05g0456100 | 3.89 | 0.00 | 2.37 | 0.00 |
| Aldo/keto reductase | Os05g0456200 | 3.40 | 0.00 | 2.57 | 0.00 |
| Early nodulin 93 | Os06g0141600 | 3.06 | 0.00 | 3.47 | 0.00 |
| Integral membrane protein | Os06g0218900 | 3.12 | 0.00 | 2.37 | 0.00 |
| Haem peroxidase | Os06g0521500 | 3.01 | 0.00 | 3.44 | 0.00 |
| Pathogenesis-related protein | Os07g0129300 | 3.08 | 0.00 | 3.44 | 0.00 |
| RLK (receptor lectin kinase) | Os07g0129800 | 4.87 | 0.00 | 3.96 | 0.00 |
| 9-cis-epoxycarotenoid dioxygenase | Os07g0154100 | 20.06 | 0.00 | 25.41 | 0.00 |
| Cloroplastosos alterados | Os07g0190000 | 4.09 | 0.00 | 4.50 | 0.00 |
| Leucine-rich repeat transmembrane kinase | Os07g0251900 | 8.22 | 0.00 | 5.96 | 0.00 |
| Leucine-rich repeat protein kinase | Os08g0201700 | 3.53 | 0.00 | 2.26 | 0.00 |
| Leucine-rich repeat protein kinase | Os08g0203400 | 7.64 | 0.00 | 6.21 | 0.00 |
| WRKY40 | Os09g0417600 | 7.64 | 0.00 | 7.21 | 0.00 |
| WRKY18 | Os09g0417800 | 6.93 | 0.00 | 7.16 | 0.00 |
| Potassium ion transmembrane transporter | Os09g0448200 | 7.71 | 0.00 | 7.06 | 0.00 |
| WAK2 | Os10g0151100 | 6.63 | 0.00 | 4.06 | 0.00 |
| Calcium-transporting ATPase | Os10g0418100 | 26.21 | 0.00 | 32.54 | 0.00 |
| Aspartyl protease | Os10g0537800 | 4.59 | 0.00 | 4.28 | 0.00 |
| Aspartyl protease | Os10g0538200 | 4.47 | 0.00 | 3.98 | 0.00 |
| DNA binding/Homeodomain | Os11g0282700 | 126.94 | 0.00 | 100.82 | 0.00 |
| Calcium-binding EF hand family protein | Os11g0600500 | 4.32 | 0.00 | 4.02 | 0.00 |
| Zinc finger | Os11g0687100 | 5.60 | 0.00 | 6.05 | 0.00 |
| Zinc finger | Os11g0702400 | 3.23 | 0.00 | 3.74 | 0.00 |
| Germin-like protein 9 | Os12g0154800 | 3.01 | 0.00 | 2.99 | 0.00 |
| AAA-ATPase 1 | Os12g0431100 | 3.10 | 0.00 | 4.24 | 0.00 |

Up-regulated genes in RCc3: OsNAC1

| | | | | | |
|---|---|---|---|---|---|
| Cytochrome P450 | Os02g0601500 | 5.42 | 0.00 | 1.86 | 0.00 |
| MtN3 | Os05g0426000 | 4.03 | 0.00 | 1.27 | 0.07 |
| Leucine-rich repeat | Os08g0202300 | 3.34 | 0.00 | 1.52 | 0.03 |
| O-methyltransferase | Os09g0344500 | 3.68 | 0.00 | 1.23 | 0.05 |
| AAA-type ATPase | Os09g0445700 | 31.09 | 0.00 | 1.15 | 0.11 |
| O-methyltransferase | Os10g0118000 | 4.39 | 0.00 | 1.50 | 0.01 |
| O-methyltransferase | Os10g0118200 | 6.36 | 0.00 | 1.30 | 0.06 |
| protein kinase | Os11g0274700 | 5.00 | 0.00 | 1.95 | 0.00 |
| Disease resistance protein | Os11g0491600 | 59.47 | 0.00 | 1.08 | 0.91 |

Up-regulated genes in GOS2: OsNAC1

| | | | | | |
|---|---|---|---|---|---|
| Aminotransferase | Os01g0729600 | 1.54 | 0.10 | 8.41 | 0.00 |
| Xyloglucosyl transferase | Os02g0280300 | −2.09 | 0.01 | 4.44 | 0.00 |
| Cinnamoyl CoA reductase 1 | Os02g0808800 | −1.23 | 0.47 | 9.51 | 0.00 |
| Downy mildew resistant 6 | Os03g0122300 | 1.78 | 0.01 | 3.01 | 0.00 |
| Proline extensin-like receptor kinase 1 | Os03g0269300 | 1.76 | 0.01 | 5.31 | 0.00 |
| WRKY1 | Os03g0335200 | 1.78 | 0.03 | 3.28 | 0.00 |
| Salt tolerance zinc finger | Os03g0437200 | −1.29 | 0.76 | 3.65 | 0.00 |
| AAA-ATPase 1 | Os03g0802400 | 1.44 | 0.15 | 3.13 | 0.00 |
| Hydrolase | Os04g0411800 | 1.81 | 0.01 | 3.30 | 0.00 |
| Hydrolase | Os04g0412000 | 1.55 | 0.03 | 3.05 | 0.00 |
| Membrane bound O-acyl transferase | Os04g0481800 | 1.92 | 0.02 | 4.22 | 0.00 |
| Cinnamyl alcohol dehydrogenase 6 | Os04g0612700 | −1.33 | 0.37 | 5.27 | 0.00 |
| Leucine-rich repeat | Os04g0621900 | −0.05 | 0.50 | 4.18 | 0.00 |
| Phosphofructokinase 3 | Os05g0194900 | 1.73 | 0.01 | 4.42 | 0.00 |
| Pyruvate decarboxylase | Os05g0469600 | 1.61 | 0.00 | 4.65 | 0.00 |
| L-lactate dehydrogenase | Os06g0104900 | 1.67 | 0.01 | 4.17 | 0.00 |
| Disease resistance protein | Os06g0279900 | −1.18 | 0.18 | 4.54 | 0.00 |
| FAD-binding domain-containing protein | Os06g0548200 | 2.00 | 0.00 | 3.78 | 0.00 |
| Universal stress protein | Os07g0673400 | 1.89 | 0.01 | 4.39 | 0.00 |
| Terpene synthase/cyclase | Os08g0167800 | 1.89 | 0.02 | 3.74 | 0.00 |
| Acidic endochitinase | Os08g0518900 | 1.95 | 0.00 | 4.03 | 0.00 |
| Pin-formed 5 | Os08g0529000 | 0.17 | 0.43 | 4.10 | 0.00 |
| Calcium-binding EF | Os09g0483100 | −0.08 | 0.94 | 6.22 | 0.00 |
| Calcium-binding EF hand | Os09g0483300 | 1.39 | 0.12 | 3.54 | 0.00 |
| Purple acid phosphatase 3 | Os10g0116800 | 1.46 | 0.21 | 3.96 | 0.00 |
| Phosphoenolpyruvate carboxykinas 1 | Os10g0204400 | 1.53 | 0.05 | 4.42 | 0.00 |
| HAT dimerization domain-containing | Os10g0567900 | −0.11 | 0.60 | 3.56 | 0.00 |
| protein | | | | | |
| Acidic endochitinase | Os11g0701000 | −1.27 | 0.65 | 3.03 | 0.00 |

^{a}Sequence identification numbers for the full-length cDNA sequences of the corresponding genes.
^{b}The mean of duplicate biological samples.
^{c}p-values were analyzed by one-way ANOVA (p < 0.01). These microarray data sets can be found at http://www.ncbi.nlm.nih.gov/geo/(Gene Expression Omnibus, GEO, Accession number)

### SEQ ID NO:s for Table A Sequences

Up-regulated genes in RCc3: OsNAC1 and GOS2: OsNAC1

| | | |
|---|---|---|
| Protein kinase | Os01g0117600 | SEQ ID NO: 48 & 49 |
| ABC transporter | Os01g0609300 | SEQ ID NO: 50 & 51 |
| Peptidase aspartic | Os01g0937500 | SEQ ID NO: 52 & 53 |
| Cytochrome P450 | Os02g0601400 | SEQ ID NO: 54 & 55 |
| WAK3 | Os02g0807900 | SEQ ID NO: 56 & 57 |
| Cinnamoyl CoA Reductase 1 | Os02g0811800 | SEQ ID NO: 58 & 59 |
| Acyl-activating enzyme | Os03g0130100 | SEQ ID NO: 60 & 61 |
| Phytosulfokine | Os03g0232400 | SEQ ID NO: 62 & 63 |
| U-box | Os03g0240600 | SEQ ID NO: 64 & 65 |
| Aspartyl protease | Os03g0318400 | SEQ ID NO: 66 & 67 |
| High affinity K+ transporter 5 | Os03g0575200 | SEQ ID NO: 68 & 69 |
| Copalyl diphosphate synthetase | Os04g0178300 | SEQ ID NO: 70 & 71 |
| RLP (receptor-like protein kinase) | Os04g0202700 | SEQ ID NO: 72 & 73 |
| MAPKKK9 | Os04g0339800 | SEQ ID NO: 74 & 75 |
| WAK2 | Os04g0365100 | SEQ ID NO: 76 & 77 |
| WAK2 | Os04g0368800 | SEQ ID NO: 78 & 79 |
| Glutamate dehydrogenase | Os04g0543900 | SEQ ID NO: 80 & 81 |
| Downy mildew resistnant 6 | Os04g0581000 | SEQ ID NO: 82 & 83 |
| Oxidoreductase, 2OG-Fe(II) oxygenase | Os04g0581100 | SEQ ID NO: 84 & 85 |
| Pyruvate kinase | Os04g0677300 | SEQ ID NO: 86 & 87 |
| Zinc finger | Os05g0404700 | SEQ ID NO: 88 & 89 |
| Aldo/keto reductase | Os05g0456100 | SEQ ID NO: 90 & 91 |
| Aldo/keto reductase | Os05g0456200 | SEQ ID NO: 92 & 93 |
| Early nodulin 93 | Os06g0141600 | SEQ ID NO: 94 & 95 |
| Integral membrane protein | Os06g0218900 | SEQ ID NO: 96 & 97 |
| Haem peroxidase | Os06g0521500 | SEQ ID NO: 98 & 99 |
| Pathogenesis-related protein | Os07g0129300 | SEQ ID NO: 100 & 101 |
| RLK (receptor lectin kinase) | Os07g0129800 | SEQ ID NO: 102 & 103 |
| 9-cis-epoxycarotenoid dioxygenase | Os07g0154100 | SEQ ID NO: 104 & 105 |
| Cloroplastosos alterados | Os07g0190000 | SEQ ID NO: 106 & 107 |
| Leucine-rich repeat transmembrane kinase | Os07g0251900 | SEQ ID NO: 108 & 109 |
| Leucine-rich repeat protein kinase | Os08g0201700 | SEQ ID NO: 110 & 111 |
| Leucine-rich repeat protein kinase | Os08g0203400 | SEQ ID NO: 112 & 113 |
| WRKY40 | Os09g0417600 | SEQ ID NO: 114 & 115 |
| WRKY18 | Os09g0417800 | SEQ ID NO: 116 & 117 |
| Potassium ion transmembrane transporter | Os09g0448200 | SEQ ID NO: 118 & 119 |
| WAK2 | Os10g0151100 | SEQ ID NO: 120 & 121 |
| Calcium-transporting ATPase | Os10g0418100 | SEQ ID NO: 122 & 123 |
| Aspartyl protease | Os10g0537800 | SEQ ID NO: 124 & 125 |
| Aspartyl protease | Os10g0538200 | SEQ ID NO: 126 & 127 |
| DNA binding/Homeodomain | Os11g0282700 | SEQ ID NO: 128 & 129 |
| Calcium-binding EF hand family protein | Os11g0600500 | SEQ ID NO: 130 & 131 |
| Zinc finger | Os11g0687100 | SEQ ID NO: 132 & 133 |
| Zinc finger | Os11g0702400 | SEQ ID NO: 134 & 135 |
| Germin-like protein 9 | Os12g0154800 | SEQ ID NO: 136 & 137 |
| AAA-ATPase 1 | Os12g0431100 | SEQ ID NO: 138 & 139 |

Up-regulated genes in RCc3: OsNAC1

| | | |
|---|---|---|
| Cytochrome P450 | Os02g0601500 | SEQ ID NO: 140 & 141 |
| MtN3 | Os05g0426000 | SEQ ID NO: 142 & 143 |
| Leucine-rich repeat | Os08g0202300 | SEQ ID NO: 144 & 145 |
| O-methyltransferase | Os09g0344500 | SEQ ID NO: 146 & 147 |
| AAA-type ATPase | Os09g0445700 | SEQ ID NO: 148 & 149 |
| O-methyltransferase | Os10g0118000 | SEQ ID NO: 150 & 151 |
| O-methyltransferase | Os10g0118200 | SEQ ID NO: 152 & 153 |
| protein kinase | Os11g0274700 | SEQ ID NO: 154 & 155 |
| Disease resistance protein | Os11g0491600 | SEQ ID NO: 156 & 157 |

Up-regulated genes in GOS2: OsNAC1

| | | |
|---|---|---|
| Aminotransferase | Os01g0729600 | SEQ ID NO: 158 & 159 |
| Xyloglucosyl transferase | Os02g0280300 | SEQ ID NO: 160 & 161 |
| Cinnamoyl CoA reductase 1 | Os02g0808800 | SEQ ID NO: 162 & 163 |
| Downy mildew resistant 6 | Os03g0122300 | SEQ ID NO: 164 & 165 |
| Proline extensin-like receptor kinase 1 | Os03g0269300 | SEQ ID NO: 166 & 167 |
| WRKY1 | Os03g0335200 | SEQ ID NO: 168 & 169 |
| Salt tolerance zinc finger | Os03g0437200 | SEQ ID NO: 170 & 171 |
| AAA-ATPase 1 | Os03g0802400 | SEQ ID NO: 172 & 173 |
| Hydrolase | Os04g0411800 | SEQ ID NO: 174 & 175 |
| Hydrolase | Os04g0412000 | SEQ ID NO: 176 |
| Membrane bound O-acyl transferase | Os04g0481800 | SEQ ID NO: 177 & 178 |
| Cinnamyl alcohol dehydrogenase 6 | Os04g0612700 | SEQ ID NO: 179 & 180 |
| Leucine-rich repeat | Os04g0621900 | SEQ ID NO: 181 & 182 |
| Phosphofructokinase 3 | Os05g0194900 | SEQ ID NO: 183 & 184 |
| Pyruvate decarboxylase | Os05g0469600 | SEQ ID NO: 185 & 186 |
| L-lactate dehydrogenase | Os06g0104900 | SEQ ID NO: 187 & 188 |
| Disease resistance protein | Os06g0279900 | SEQ ID NO: 189 & 190 |
| FAD-binding domain-containing protein | Os06g0548200 | SEQ ID NO: 191 & 192 |
| Universal stress protein | Os07g0673400 | SEQ ID NO: 193 & 194 |
| Terpene synthase/cyclase | Os08g0167800 | SEQ ID NO: 195 & 196 |
| Acidic endochitinase | Os08g0518900 | SEQ ID NO: 197 & 198 |
| Pin-formed 5 | Os08g0529000 | SEQ ID NO: 199 & 200 |
| Calcium-binding EF | Os09g0483100 | SEQ ID NO: 201 & 201 |
| Calcium-binding EF hand | Os09g0483300 | SEQ ID NO: 203 & 204 |
| Purple acid phosphatase 3 | Os10g0116800 | SEQ ID NO: 205 & 206 |
| Phosphoenolpyruvate carboxykinas 1 | Os10g0204400 | SEQ ID NO: 207 & 208 |
| HAT dimerization domain-containing protein | Os10g0567900 | SEQ ID NO: 209 & 210 |
| Acidic endochitinase | Os11g0701000 | SEQ ID NO: 211 & 212 |

### Example 4

### Transgenic Overexpression of OsNAC5 Increased Plant Tolerance to Drought and High-Salinity Conditions

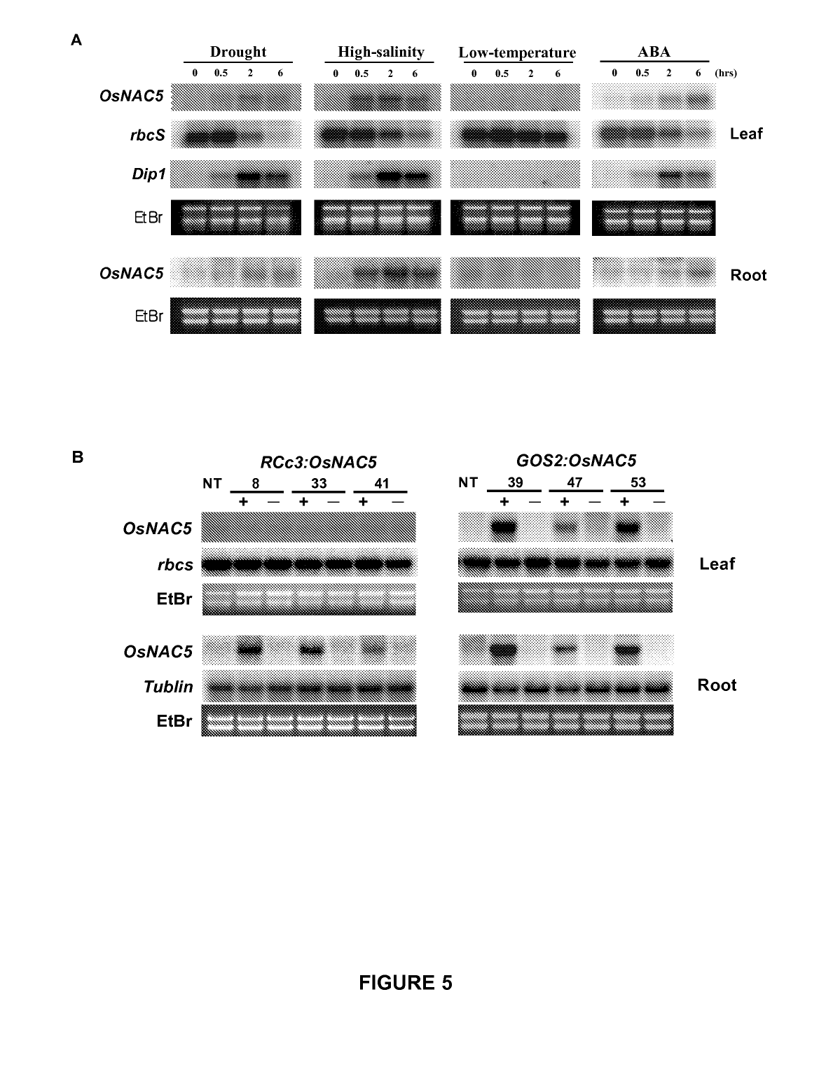

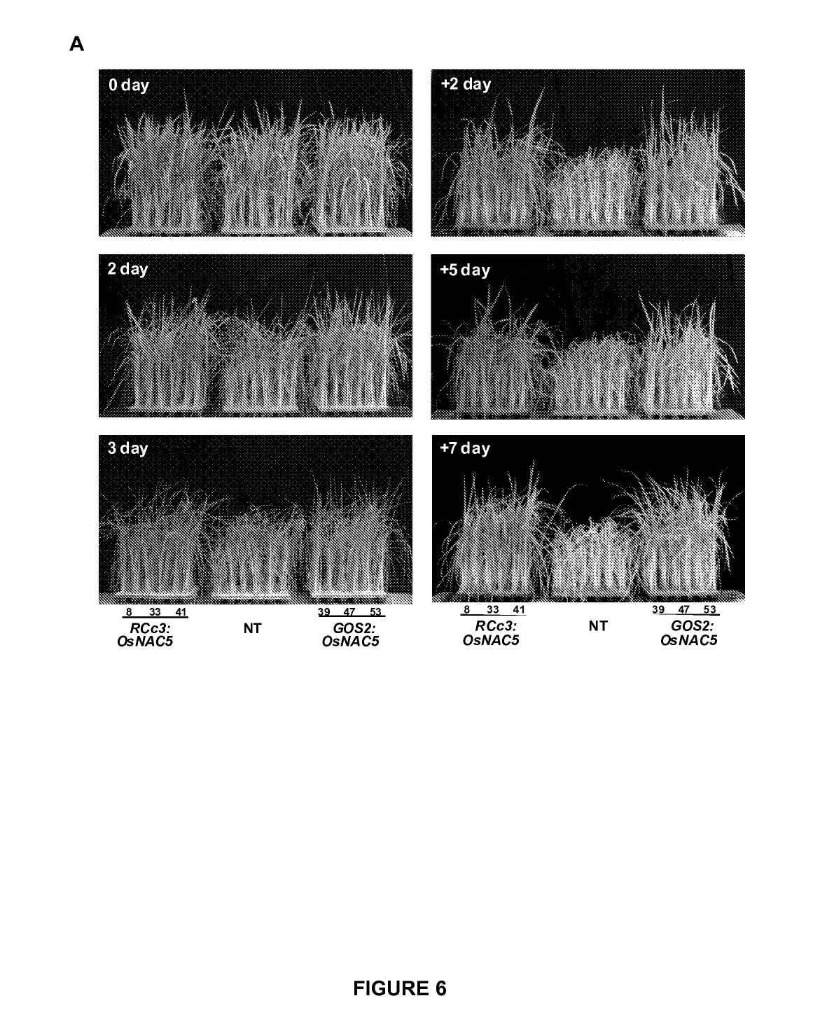

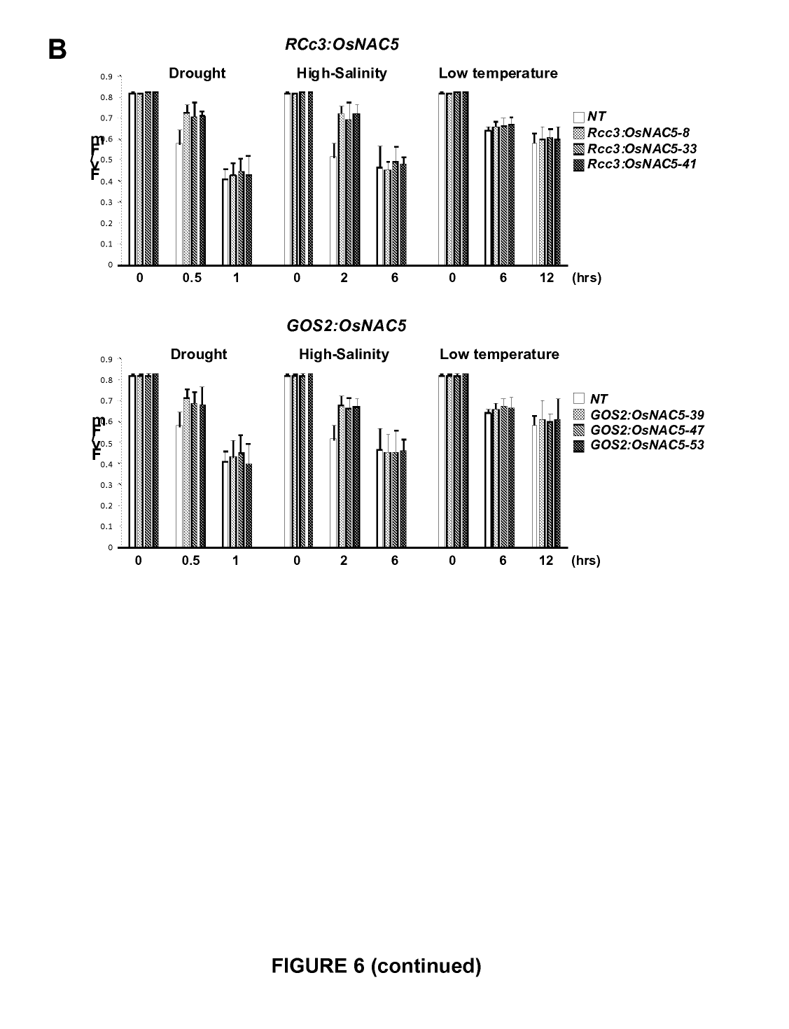

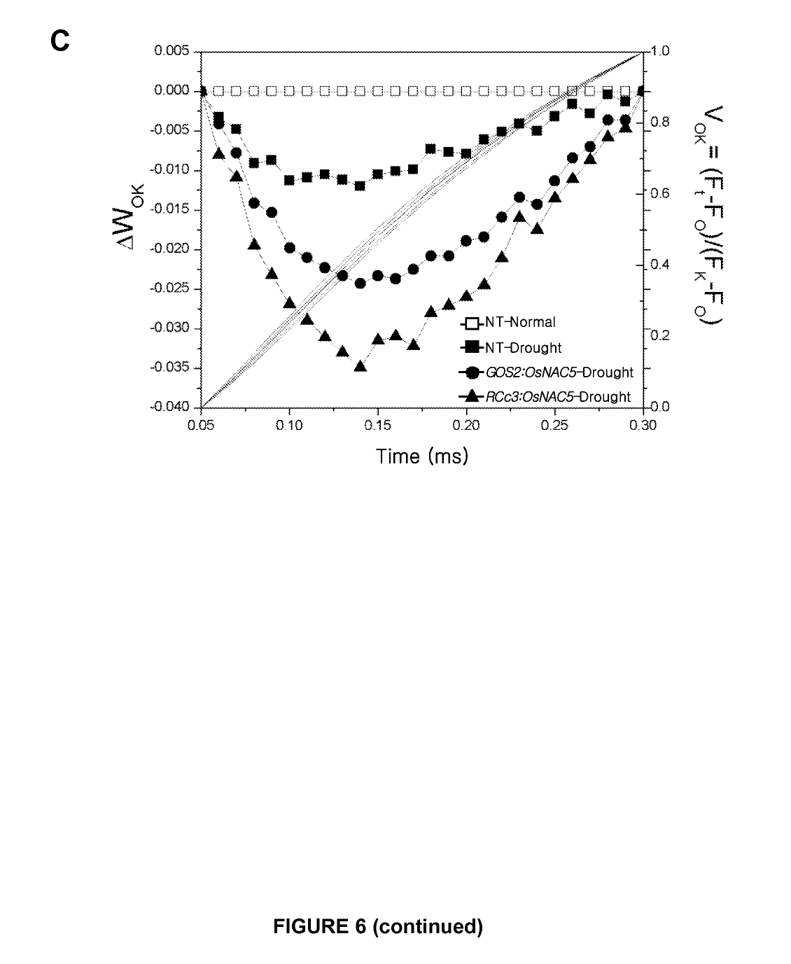

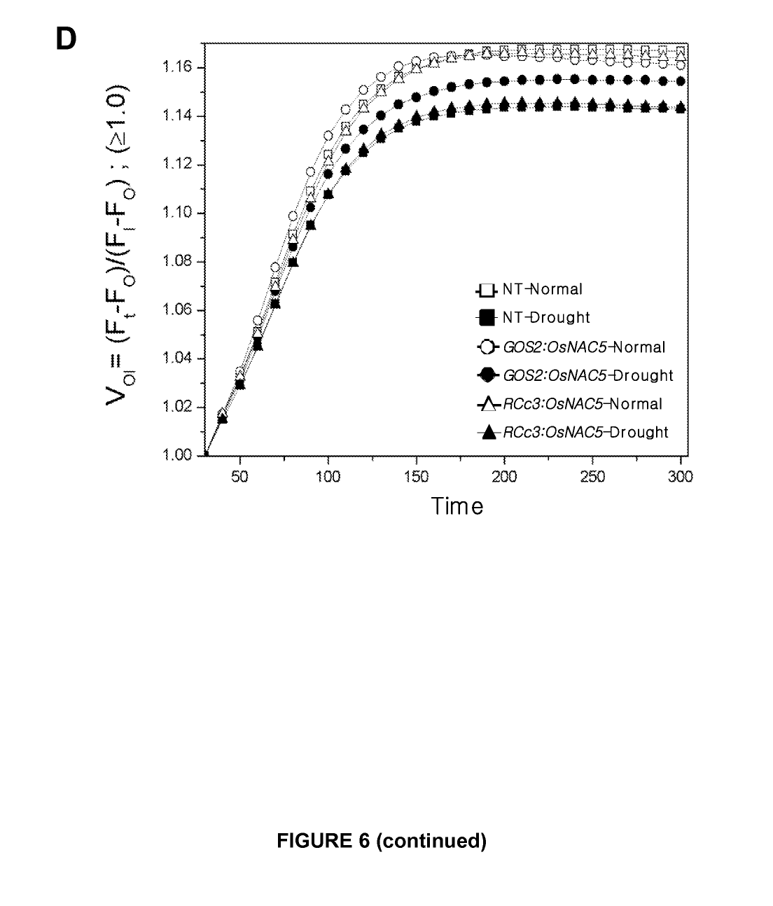

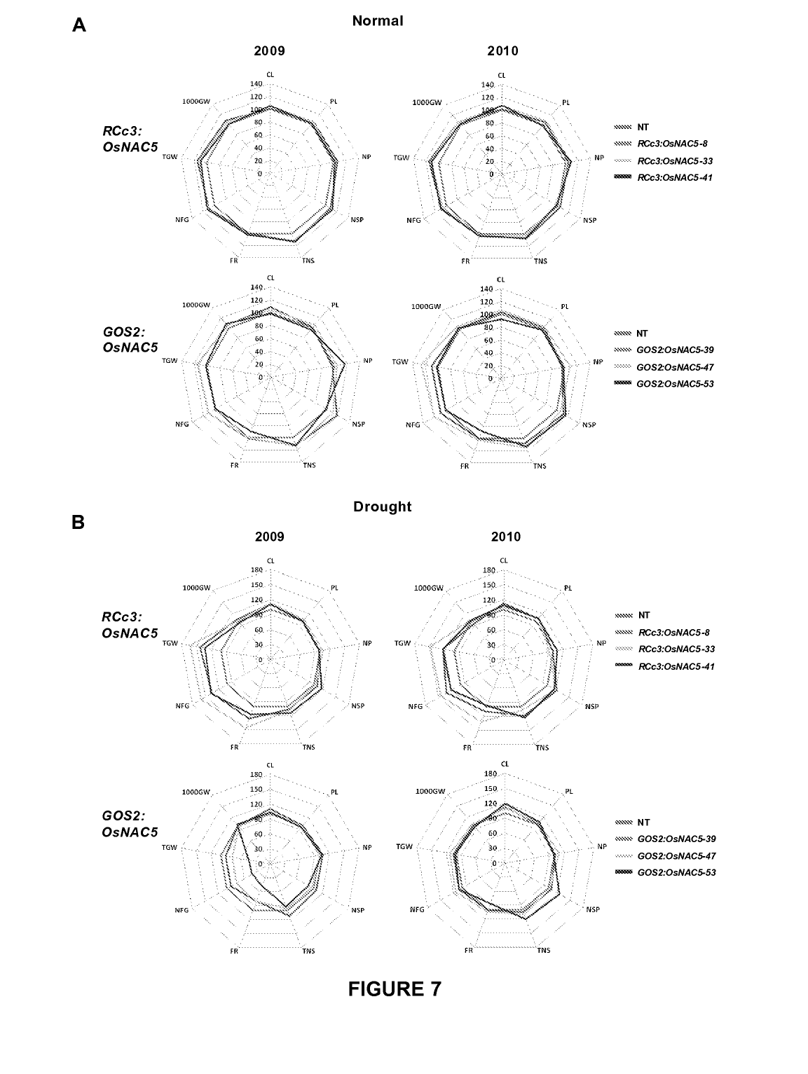

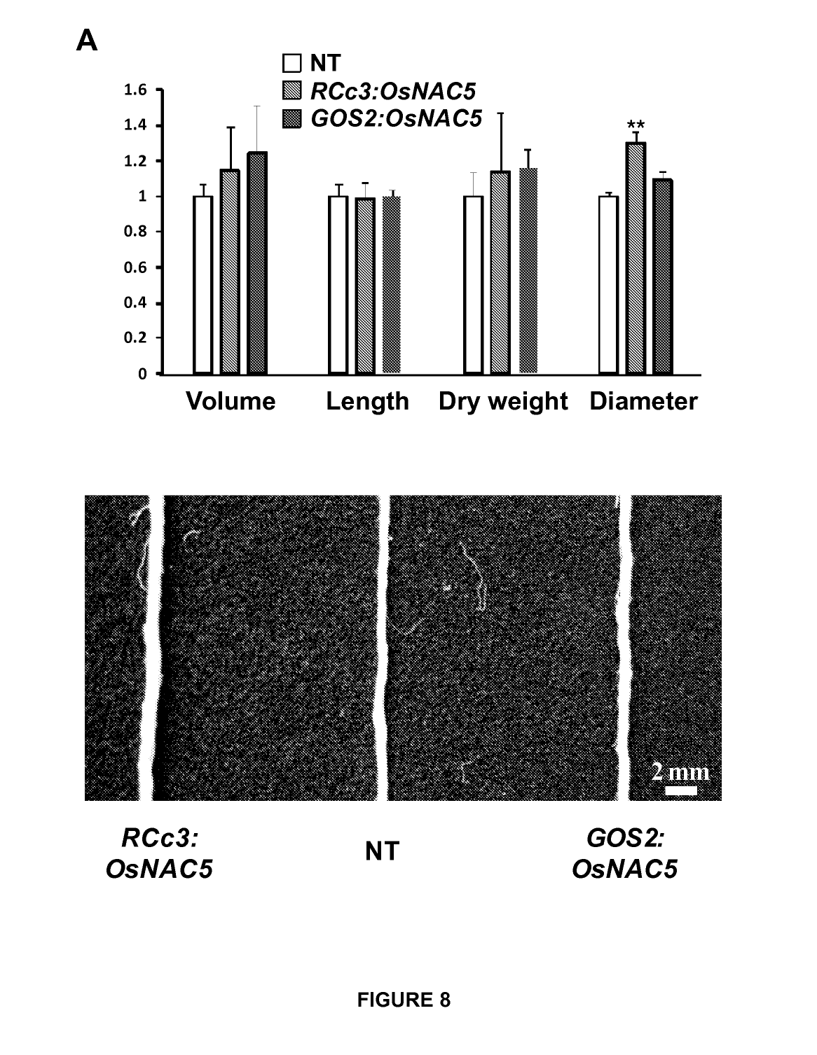

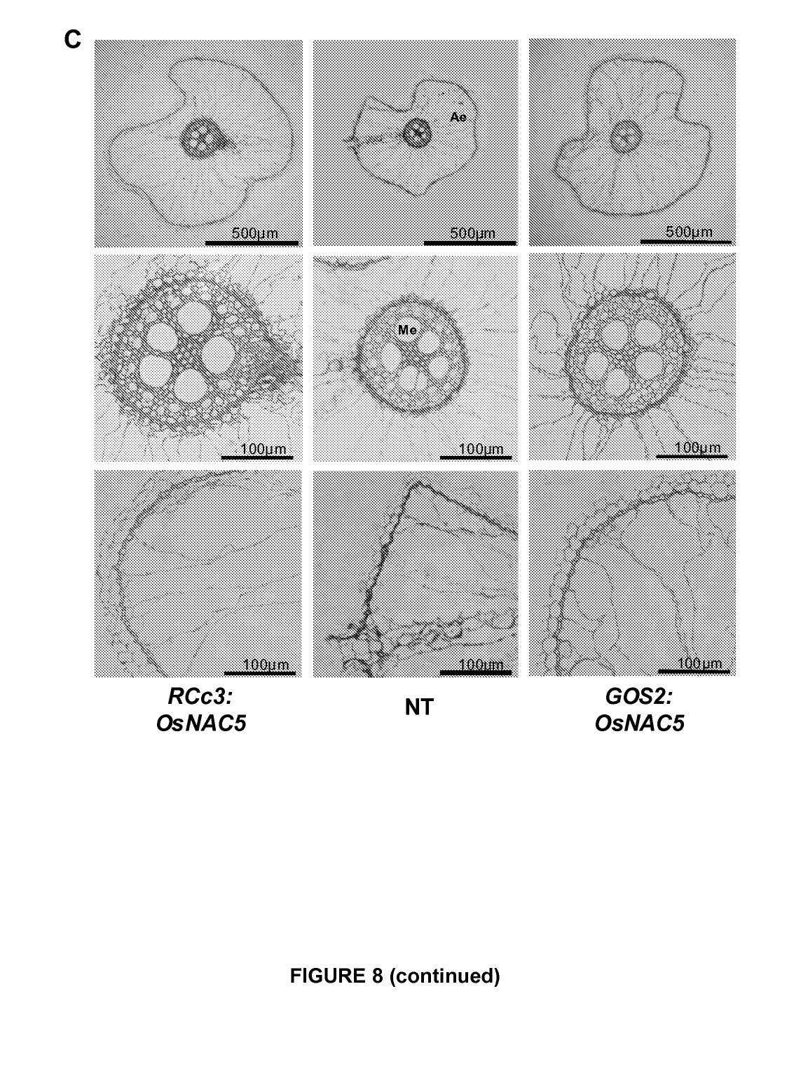

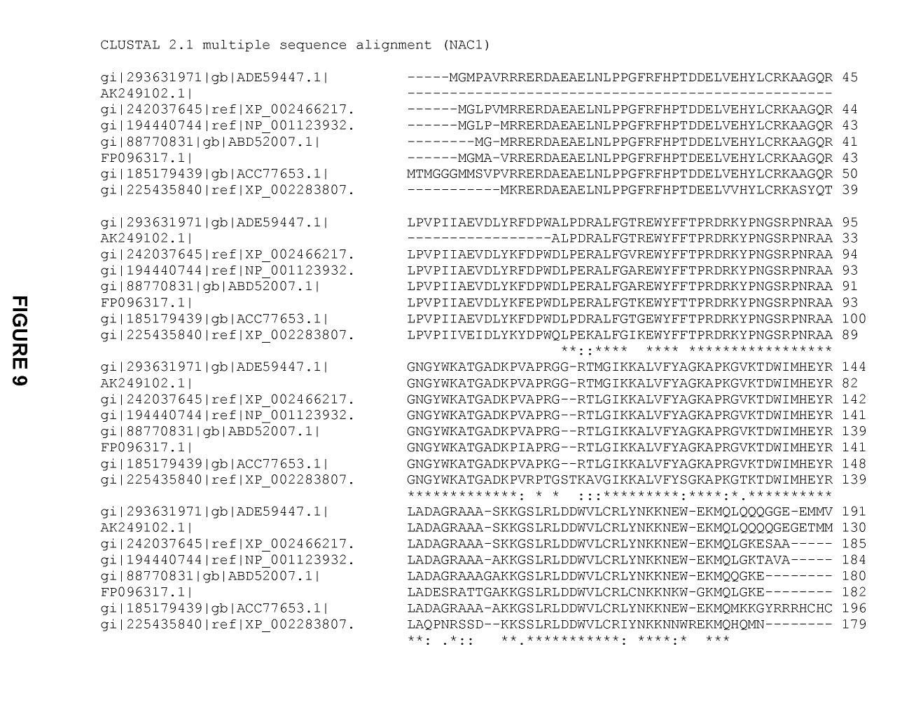

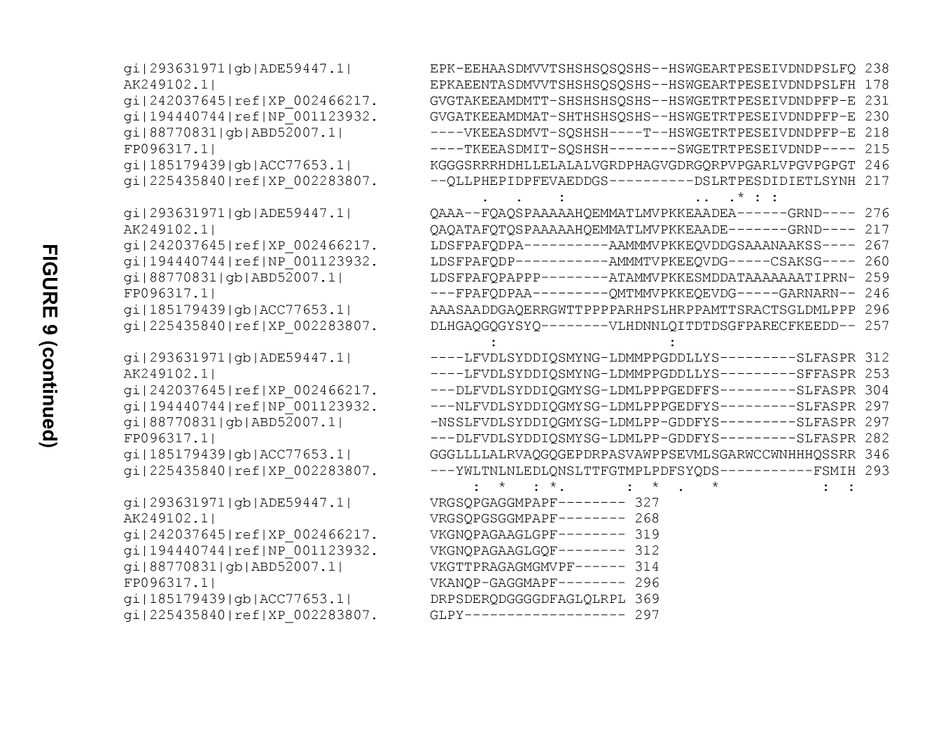

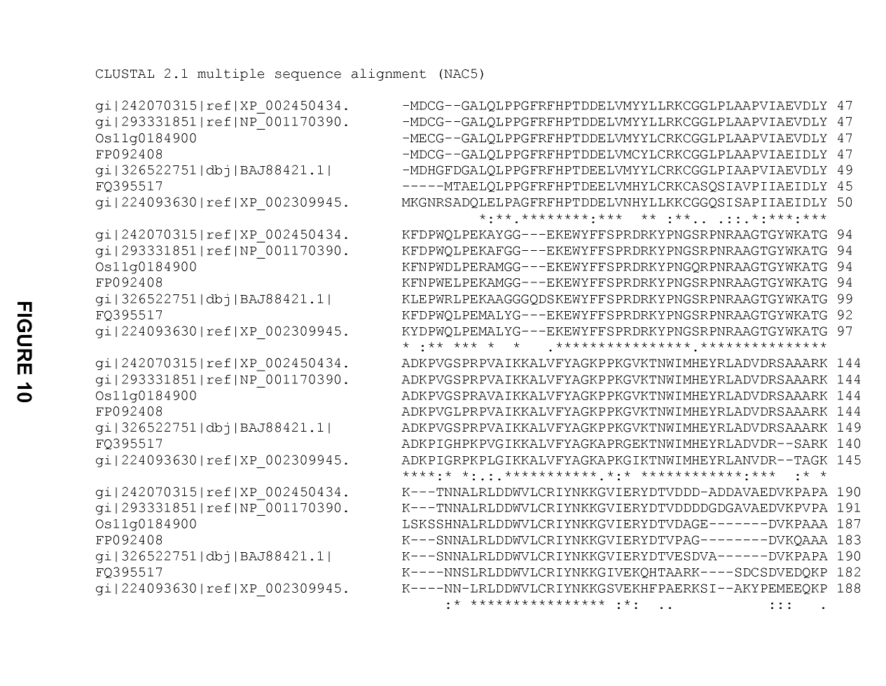

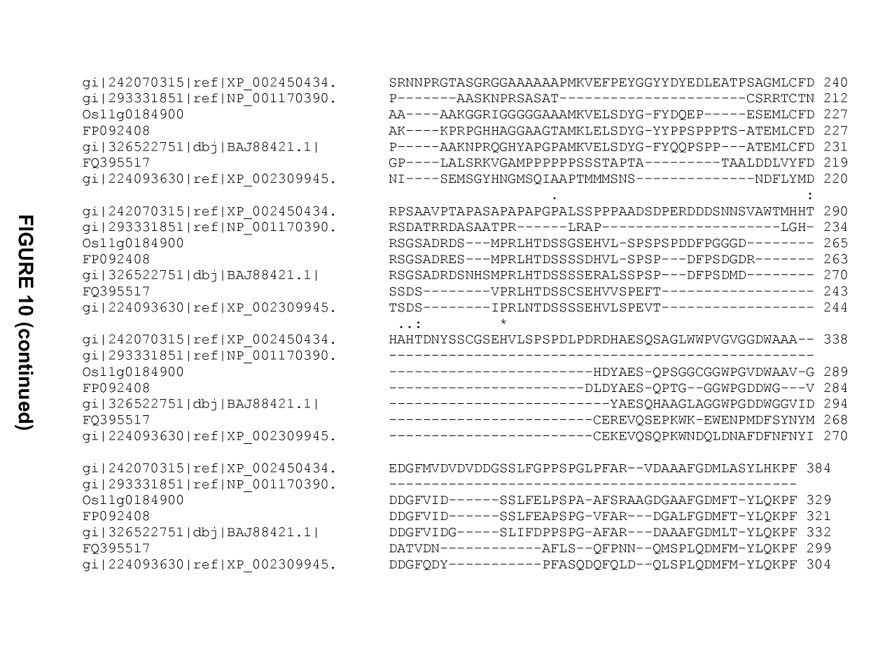

To investigate the transcript levels of OsNAC5 under stress conditions, we performed RNA-gel blot analysis using total RNAs from leaf and root tissues of 14-d-old rice seedlings exposed to high salinity, drought, ABA and low temperature (FIG. 5A). Expression of OsNAC5 in both leaf and root tissues was significantly induced by treatments with drought, high-salinity and ABA, but not with low temperature conditions. Transcript levels of OsNAC5 started to increase at 0.5 h after drought and salt treatments and peaked at 2 h of the stress administration while the transcript levels gradually increased up to 6 h upon treatments with exogenous ABA.

To overexpress OsNAC5 in transgenic rice plants, two expression vectors, RCc3:OsNAC5 and GOS2:OsNAC5, were made by fusing cDNA of OsNAC5 with the RCc3 (Xu et al., 1995) and the GOS2 (de Pater et al., 1992) for a root-specific and a conserved expression, respectively. The expression vectors were transformed into rice (Oryza sativa cv Nipponbare) using the Agrobacterium-mediated method (Hiei et al., 1994), producing 15-20 transgenic plants per construct. T₁₋₆ seeds from transgenic lines that grew normal without stunting were collected and three independent T₅₋₆ homozygous lines of both RCc3:OsNAC1 and GOS2:OsNAC1 plants were selected for further analysis. To determine expression levels of OsNAC5 in the transgenic plants, RNA-gel blot analysis was carried out using total RNAs from leaf and root tissues of 14-d-old seedlings grown under normal growth conditions. Increased levels of OsNAC5 expression were detected only in roots of the RCc3:OsNAC5 plants and in both leaves and roots of the GOS2:OsNAC5 plants, but not in nontransgenic (NT) and nullizygous (segregants without transgene) plants (FIG. 5B). To evaluate tolerance of transgenic plants to drought stress, one-month-old transgenic and NT control plants were treated with drought stress by withholding water in the greenhouse. In the time course of drought treatments, both transgenic plants perform better than NT controls showing delayed symptoms of stress-induced damages, such as wilting and leaf rolling with concomitant loss of chlorophylls (FIG. 6A). The transgenic plants also recovered better during re-watering up to 7 d. The survival rates of transgenic plants ranged from 60 to 80% while NT control plants had no signs of recovery.

To further verify stress tolerance of the transgenic plants, we measured alterations in Fv/Fm values, an indicator of the photochemical efficiency of photosystem II (PSII) in a dark-adapted state. The leaf discs of two-weeks-old transgenic and NT control plants were treated with drought, high-salinity and low temperature for the indicated times. The Fv/Fm values of non-stressed plants were approximately 0.8. At the initial stage of drought (0.5 h) and high-salinity (2 h) conditions, Fv/Fm levels of the RCc3:OsNAC5 and GOS2:OsNAC5 plants were higher by 15-22% than those of NT controls (FIG. 6B). Under extended drought (2 h) and high-salinity (6 h) stress as well as low temperature conditions, however, the levels remained similar to those of NT controls, suggesting a moderate level of tolerance of the transgenic plants. The JIP test provides an alternative way of measuring stress tolerance by analyzing the chlorophyll a fluorescence transients between 50 μs and 300 μs after illumination of dark-adapted plants (Redillas et al., 2011a and 2011b). The JIP test carries information regarding the connectivity between the antennas of the PSII units. This connectivity can be illustrated by the difference kinetics revealing the so called L-band. This band is negative (or positive) when the connectivity of the plants is higher (or lower) than that of untreated NT controls. This connectivity is undetectable using the Fᵥ/Fₘ, analysis which also measures the chlorophyll a fluorescence of plants. We performed the JIP test on the plants at the reproductive stage, revealing that both transgenic plants had higher connectivity than NT controls under drought conditions (FIGS. 6C and D). More specifically, the connectivity is higher in the RCc3:OsNAC5 plants followed by the GOS2:OsNAC5 plants over NT controls, revealing differences in drought tolerance at the reproductive stage.

TABLE III
Agronomic traits of the RCc3: OsNAC5 and GOS2: OsNAC5
transgenic rice plants under normal field conditions

| Construct | | | |
|---|---|---|---|
| Panicle | No. of | No. of | No. of total |
| length | Panicles | Spikelets | spikelets |
| (cm) | (/hill) | (/panicle) | (/hill) |

| Normal | 2009 | 2010 | 2009 | 2010 | 2009 | 2010 | 2009 | 2010 |
|---|---|---|---|---|---|---|---|---|
| NT (Nipponbare) | 19.30 | 21.03 | 10.10 | 13.77 | 88.98 | 107.65 | 909.00 | 1468.23 |
| RCc3: OsNAC5-8 | 20.25 * | 22.07 * | 10.77 | 14.37 | 96.67 * | 112.45 | 1036.07 * | 1591.03 |
| % Δ | 4.92 | 4.91 | 6.60 | 4.36 | 8.64 | 4.46 | 13.98 | 8.36 |
| P-val | 0.00 | 0.01 | 0.14 | 0.41 | 0.01 | 0.17 | 0.00 | 0.10 |
| RCc3: OsNAC5-33 | 20.24 * | 22.63 * | 10.30 | 14.80 | 99.41 * | 102.75 | 1010.41 * | 1523.50 |
| % Δ | 4.87 | 7.61 | 1.94 | 7.51 | 11.72 | −4.56 | 11.16 | 3.76 |
| P-val | 0.00 | 0.00 | 0.67 | 0.16 | 0.00 | 0.16 | 0.01 | 0.45 |
| RCc3: OsNAC5-41 | 19.54 | 20.73 | 10.35 | 14.97 | 100.07 * | 105.65 | 1029.88 * | 1566.53 |
| % Δ | 1.24 | −1.43 | 2.44 | 8.72 | 12.47 | −1.86 | 13.30 | 6.70 |
| P-val | 0.43 | 0.43 | 0.59 | 0.10 | 0.00 | 0.56 | 0.00 | 0.18 |
| NT (Nipponbare) | 19.30 | 21.03 | 10.10 | 13.77 | 88.98 | 107.65 | 909.00 | 1468.23 |
| GOS2: OsNAC5-39 | 19.47 | 21.83 * | 9.80 | 13.30 | 105.14 * | 120.56 * | 1024.03 * | 1591.77 |
| % Δ | 0.86 | 3.80 | −2.97 | −3.39 | 18.17 | 11.99 | 12.65 | 8.41 |
| P-val | 0.57 | 0.02 | 0.50 | 0.56 | 0.00 | 0.00 | 0.00 | 0.20 |
| GOS2: OsNAC5-47 | 20.47 * | 22.07 * | 10.47 | 15.00 | 98.80 * | 114.03 | 1032.57 * | 1699.30 * |
| % Δ | 6.04 | 4.91 | 3.63 | 8.96 | 11.04 | 5.93 | 13.59 | 15.74 |
| P-val | 0.00 | 0.00 | 0.41 | 0.13 | 0.00 | 0.10 | 0.00 | 0.02 |
| GOS2: OsNAC5-53 | 18.57 * | 20.80 | 11.73 * | 13.50 | 87.59 | 124.66 * | 1022.17 * | 1670.43 * |
| % Δ | −3.80 | −1.11 | 16.17 | −1.94 | −1.57 | 15.80 | 12.45 | 13.77 |
| P-val | 0.01 | 0.50 | 0.00 | 0.74 | 0.58 | 0.00 | 0.00 | 0.04 |

| | Construct | | | | | | | |
|---|---|---|---|---|---|---|---|---|
| | Filling | | No. of filled | | Total grain | | 1000 grain | |
| | rate | | spikelets | | weight | | weight | |
| | (%) | | (/hill) | | (g) | | (g) | |
| Normal | 2009 | 2010 | 2009 | 2010 | 2009 | 2010 | 2009 | 2010 |
| NT (Nipponbare) | 91.29 | 82.74 | 846.60 | 1215.23 | 21.41 | 27.82 | 24.49 | 22.92 |
| RCc3: OsNAC5-8 | 90.22 | 82.77 | 933.37 * | 1316.50 | 24.52 * | 32.00 * | 26.30 * | 24.34 * |
| % Δ | −1.17 | 0.04 | 10.25 | 8.33 | 14.52 | 15.01 | 7.39 | 6.20 |
| P-val | 0.15 | 0.98 | 0.01 | 0.13 | 0.00 | 0.01 | 0.00 | 0.00 |
| RCc3: OsNAC5-33 | 93.42 * | 84.26 | 943.81 * | 1287.20 | 23.99 * | 31.40 * | 25.43 * | 24.31 * |
| % Δ | 2.33 | 1.83 | 11.48 | 5.92 | 12.01 | 12.85 | 3.85 | 6.06 |
| P-val | 0.01 | 0.28 | 0.01 | 0.28 | 0.01 | 0.03 | 0.00 | 0.00 |
| RCc3: OsNAC5-41 | 92.83 * | 85.20 | 955.54 * | 1333.07 | 23.41 * | 31.00 * | 24.51 | 23.32 |
| % Δ | 1.69 | 2.98 | 12.87 | 9.70 | 9.31 | 11.42 | 0.08 | 1.75 |
| P-val | 0.05 | 0.08 | 0.00 | 0.08 | 0.04 | 0.05 | 0.93 | 0.30 |
| NT (Nipponbare) | 91.29 | 82.74 | 846.60 | 1215.23 | 21.41 | 27.82 | 24.49 | 22.92 |
| GOS2: OsNAC5-39 | 92.05 | 83.11 | 941.77 * | 1322.40 | 24.47 * | 30.51 | 25.69 * | 23.28 |
| % Δ | 0.84 | 0.45 | 11.24 | 8.82 | 14.26 | 9.66 | 4.89 | 1.57 |
| P-val | 0.44 | 0.77 | 0.01 | 0.19 | 0.00 | 0.16 | 0.00 | 0.51 |
| GOS2: OsNAC5-47 | 90.84 | 85.28 * | 941.50 * | 1457.33 * | 24.38 * | 35.20 * | 25.87 * | 24.22 * |
| % Δ | −0.50 | 3.08 | 11.21 | 19.92 | 13.84 | 26.51 | 5.62 | 5.67 |
| P-val | 0.64 | 0.05 | 0.01 | 0.00 | 0.00 | 0.00 | 0.00 | 0.02 |
| GOS2: OsNAC5-53 | 81.40 * | 72.81 * | 830.63 | 1211.77 | 21.91 | 28.30 | 26.43 * | 23.61 |
| % Δ | −10.84 | −12.00 | −1.89 | −0.29 | 2.32 | 1.73 | 7.93 | 3.01 |
| P-val | 0.00 | 0.00 | 0.66 | 0.97 | 0.62 | 0.80 | 0.00 | 0.21 |

Each parameter value represents the mean ± SD (n = 30) for RCc3: OsNAC5 and GOS2: OsNAC5 plants and respective NT controls.
Percentage differences (% Δ) between the values for the RCc3: OsNAC5 and GOS2: OsNAC5 plants and respective NT controls are presented.
An asterisk (*) indicates a significant difference (p < 0.05).

### Example 5

### Overexpression of OsNAC5 Increases Grain Yield Under Both Normal and Drought Conditions

Field performance of RCc3:OsNAC5 and GOS2:OsNAC5 plants were evaluated for two cultivating seasons in a paddy field under normal and drought conditions. Three independent T₅ (2009) and T₆ (2010) homozygous lines of RCc3:OsNAC5 and GOS2:OsNAC5 plants, together with non-transgenic (NT) controls, were transplanted to a paddy field and grown to maturity. Yield parameters were scored for 30 plants per transgenic line from three replicates. Data sets from two years of field test were generally consistent and total grain weights of the RCc3:OsNAC5 and the GOS2:OsNAC5 plants were increased by 9-15% and 13-26%, respectively. The increased total grain weight in both transgenic plants was coupled with the increased number of spikelet per panicle and total number of spikelet with a filling rate similar to that of NT controls (FIG. 7A; Table III). To test the transgenic plants under drought conditions, three independent T₅ (2009) and T₆ (2010) lines of RCc3:OsNAC5 and GOS2:OsNAC5 plants were transplanted to a refined field equipped with a movable rain-off shelter. Plants were exposed to drought stress at the panicle heading stage (from 10 d before heading to 10 d after heading). After stressed until complete leaf-rolling, plants were irrigated overnight and immediately subjected again to the second round of drought treatments until complete leaf-rolling. Upon completion of drought treatments, plants were irrigated to allow recovery at the seed maturation stages. The level of drought stress imposed under the rain-off shelter was equivalent to those that give 40% of total grain weight obtained under normal growth conditions, which was evidenced by the difference in levels of total grain weight of NT plants between the normal and drought conditions (Tables III and IV). Statistical analysis of the yield parameters scored for two cultivating seasons showed that the decrease in grain yield under drought conditions was significantly smaller in the RCc3:OsNAC5 plants than that observed in either GOS2:OsNAC5 or NT controls. Specifically, in the drought-treated RCc3:OsNAC5 plants, the number of spikelet and/or filling rate were higher than in the drought-treated NT plants, which increased total grain weight by 33-63% (2009) and 22-48% (2010) depending on transgenic line (FIG. 7B; Table III). In the drought-treated GOS2:OsNAC5 plants, in contrast, the total grain weight was reduced (2009) than or remained similar (2010) to the drought-treated NT controls. Given similar levels of drought tolerance during the vegetative stage in the RCc3:OsNAC5 and GOS2:OsNAC5 plants, the differences in total grain weight under field drought conditions were rather unexpected. These observations prompted us to examine the root architecture of transgenic plants. We measured root volume, length, dry weight and diameter of RCc3:OsNAC5, GOS2:OsNAC5 and NT plants grown to the heading stage of reproduction. As shown in FIGS. 4A and B, root diameter of the RCc3:OsNAC5 and GOS2:OsNAC5 plants was larger by 30% and 10% than that of NT control plants, respectively. Microscopic analysis of cross-sectioned roots revealed that the increase in root diameter was due to the enlarged stele and aerenchyma of RCc3:OsNAC5 roots. In particular, the metaxylem (Me), a major portion of stele, and the aerenchyma (Ae), a tissue resulted from cortical cell death, were bigger in RCc3:OsNAC5 and GOS2:OsNAC5 roots as compared to NT roots (FIG. 8C). Size of metaxylem and aerenchyma had been previously correlated with drought tolerance at the reproductive stage (Yambao et al., 1992; Zhu et al., 2010). The volume and dry weight of the RCc3:OsNAC5 and GOS2:OsNAC5 roots were also increased, suggesting that these parameters together with diameter contributed to the increase in grain yield of the transgenic plants under normal and/or drought conditions.

TABLE IV
Agronomic traits of the RCc3: OsNAC5 and GOS2: OsNAC5
transgenic rice plants under field drought conditions

| Construct | | | |
|---|---|---|---|
| Panicle | No. of | No. of | No. of total |
| length | Panicles | Spikelets | spikelets |
| (cm) | (/hill) | (/panicle) | (/hill) |

| Drought | 2009 | 2010 | 2009 | 2010 | 2009 | 2010 | 2009 | 2010 |
|---|---|---|---|---|---|---|---|---|
| NT (Nipponbare) | 18.92 | 18.58 | 11.00 | 12.06 | 79.91 | 90.05 | 873.00 | 1089.50 |
| RCc3: OsNAC5-8 | 19.00 | 20.06 * | 10.83 | 12.00 | 86.20 | 110.04 * | 930.25 | 1296.61 * |
| % Δ | 0.44 | 7.92 | −1.52 | −0.46 | 7.87 | 22.21 | 6.56 | 19.01 |
| P-val | 0.84 | 0.00 | 0.75 | 0.20 | 0.07 | 0.02 | 0.21 | 0.01 |
| RCc3: OsNAC5-33 | 19.63 | 20.28 * | 11.65 | 11.11 | 82.19 | 107.67 * | 953.35 | 1168.83 * |
| % Δ | 3.74 | 9.12 | 5.93 | −7.83 | 2.85 | 19.57 | 9.20 | 7.28 |
| P-val | 0.09 | 0.00 | 0.21 | 0.94 | 0.51 | 0.01 | 0.08 | 0.01 |
| RCc3: OsNAC5-41 | 18.75 | 19.92 * | 10.88 | 13.06 | 93.41 * | 105.11 * | 1000.21 * | 1341.22 * |
| % Δ | −0.88 | 7.17 | −1.14 | 8.29 | 16.90 | 16.73 | 14.57 | 23.10 |
| P-val | 0.68 | 0.01 | 0.81 | 0.17 | 0.00 | 0.04 | 0.01 | 0.00 |
| NT (Nipponbare) | 18.92 | 18.58 | 11.00 | 12.06 | 79.91 | 90.05 | 873.00 | 1089.50 |
| GOS2: OsNAC5-39 | 19.00 | 19.00 | 11.70 | 12.22 | 84.52 | 95.52 | 977.96 * | 1144.17 |
| % Δ | 0.44 | 2.24 | 6.32 | 1.38 | 5.76 | 6.08 | 12.02 | 5.02 |
| P-val | 0.83 | 0.44 | 0.18 | 0.78 | 0.23 | 0.51 | 0.02 | 0.55 |
| GOS2: OsNAC5-47 | 18.58 | 19.50 | 11.00 | 13.17 | 76.82 | 92.34 | 834.92 | 1195.22 |
| % Δ | −1.76 | 4.93 | 0.00 | 9.22 | −3.86 | 2.55 | −4.36 | 9.70 |
| P-val | 0.38 | 0.09 | 1.00 | 0.07 | 0.42 | 0.78 | 0.40 | 0.25 |
| GOS2: OsNAC5-53 | 18.00 * | 19.86 * | 11.43 | 11.83 | 70.40 * | 112.85 * | 799.87 | 1304.78 * |
| % Δ | −4.85 | 6.88 | 3.95 | −1.84 | −11.90 | 25.32 | −8.38 | 19.76 |
| P-val | 0.02 | 0.02 | 0.40 | 0.71 | 0.02 | 0.01 | 0.11 | 0.02 |

| | Construct | | | | | | | |
|---|---|---|---|---|---|---|---|---|
| | Filling | | No. of filled | | Total grain | | 1000 grain | |
| | rate | | spikelets | | weight | | weight | |
| | (%) | | (/hill) | | (g) | | (g) | |
| Drought | 2009 | 2010 | 2009 | 2010 | 2009 | 2010 | 2009 | 2010 |
| NT (Nipponbare) | 47.03 | 47.62 | 406.79 | 515.33 | 8.55 | 10.09 | 21.12 | 19.49 |
| RCc3: OsNAC5-8 | 59.43 * | 52.25 * | 549.71 * | 677.89 * | 12.22 * | 12.40 * | 22.11 | 18.20 |
| % Δ | 26.36 | 9.73 | 35.13 | 31.54 | 42.81 | 22.91 | 4.71 | −6.59 |
| P-val | 0.00 | 0.00 | 0.00 | 0.00 | 0.00 | 0.00 | 0.05 | 0.309 |
| RCc3: OsNAC5-33 | 68.63 * | 63.05 * | 651.91 * | 742.78 * | 13.97 * | 14.97 * | 21.37 | 20.18 |
| % Δ | 45.91 | 32.40 | 60.26 | 44.14 | 63.30 | 48.35 | 1.21 | 3.55 |
| P-val | 0.00 | 0.00 | 0.00 | 0.02 | 0.00 | 0.00 | 0.62 | 0.062 |
| RCc3: OsNAC5-41 | 54.95 * | 46.47 | 556.17 * | 625.83 * | 11.39 * | 12.38 * | 20.20 | 20.02 |
| % Δ | 16.83 | −2.41 | 36.72 | 21.44 | 33.16 | 22.69 | −4.35 | 2.74 |
| P-val | 0.01 | 0.79 | 0.00 | 0.01 | 0.00 | 0.00 | 0.08 | 0.432 |
| NT (Nipponbare) | 47.03 | 47.62 | 406.79 | 515.33 | 8.55 | 10.09 | 21.12 | 19.49 |
| GOS2: OsNAC5-39 | 37.65 | 47.88 | 367.16 | 550.00 | 7.70 | 10.64 | 21.04 | 19.34 |
| % Δ | −19.95 | 0.95 | −9.74 | 6.73 | −10.01 | 5.45 | −0.35 | −0.75 |
| P-val | 0.05 | 0.96 | 0.39 | 0.64 | 0.38 | 0.71 | 0.90 | 0.886 |
| GOS2: OsNAC5-47 | 37.81 * | 49.59 | 317.83 * | 595.11 | 6.52 * | 11.31 | 20.45 | 19.93 |
| % Δ | −19.61 | 4.15 | −21.87 | 15.48 | −23.75 | 12.11 | −3.14 | 2.25 |
| P-val | 0.04 | 0.68 | 0.04 | 0.28 | 0.03 | 0.41 | 0.23 | 0.56 |
| GOS2: OsNAC5-53 | 22.18 * | 41.31 | 170.74 * | 550.78 | 3.72 * | 10.28 | 21.71 | 18.46 |
| % Δ | −52.84 | −13.25 | −58.03 | 6.88 | −56.54 | 1.93 | 2.79 | −5.27 |
| P-val | 0.00 | 0.18 | 0.00 | 0.63 | 0.00 | 0.90 | 0.30 | 0.228 |

Each parameter value represents the mean ± SD (n = 30) for RCc3: OsNAC5 and GOS2: OsNAC5 plants and respective NT controls.
Percentage differences (% Δ) between the values for the RCc3: OsNAC5 and GOS2: OsNAC5 plants and respective NT controls are presented.
An asterisk (*) indicates a significant difference (p < 0.05).

### Example 6

### Identification of Genes Up-Regulated Following OsNAC5 Overexpression

To identify genes that are up-regulated by the overexpression of OsNAC5, we performed expression profiling of the RCc3:OsNAC5 and GOS2:OsNAC5 plants in comparison with NT controls under normal growth conditions. This profiling was conducted using the Rice 3′-tiling microarray with RNA samples extracted from roots of 14-d-old plants grown under normal conditions. Each data set was obtained from two biological replicates. Statistical analysis using one-way ANOVA identified 25 target genes that were up-regulated following OsNAC5 overexpression by more than 3-fold in both transgenic roots as compared to NT controls (P<0.05). Also identified in the same analysis were 19 and 18 target genes that were up-regulated specifically in the RCc3:OsNAC5 and GOS2:OsNAC5 roots, respectively (Table B). Microarray experiments previously performed (GEO accession number GSE31874) revealed a total of 22 out of 62 target genes (7, 8 and 7 genes for common, RCc3:OsNAC5-specific and GOS2:OsNAC5-specific, respectively) to be stress-inducible under drought, high-salinity, cold and ABA (Table B). In addition, GLP (Yin et al., 2009), PDX (Titiz et al., 2006), MERI5 (Verica and Medford, 1997) and O-methyltransferase (Held et al., 1993), genes involved in cell growth and development, were up-regulated specifically in RCc3:OsNAC5 roots, suggesting their role(s) in alteration of root architecture. Those target genes that are either commonly or specifically up-regulated in OsNAC5 transgenic roots may account for the altered root architecture and thereby the increased drought tolerance phenotype.

The microoarray experiments identified 19 and 18 root-expressed genes that were up-regulated specifically in the RCc3:OsNAC5 and the GOS2:OsNAC5 plants, respectively, in addition to the 25 root-expressed genes that were up-regulated commonly in both plants. A number of genes that function in stress responses were up-regulated in both transgenic roots. These include cytochrome P450, ZIM, oxidase, stress response protein and heat shock protein. Also identified in both transgenic roots were transcription factors, such as WRKY, bZIP, and Zinc finger and reactive oxygen species scavenging systems such as multicopper oxidase, chitinase and glycosyl hydrolase. Increased expression of those target genes could have contributed to enhanced tolerance to drought conditions. Of the target genes specifically up-regulated in RCc3:OsNAC5 roots were GLP, PDX, MERI5 and O-methyltransferase that are known to function in cell growth and development. Arabidopsis GLP4, which specifically binds to IAA, was proposed to regulate cell growth (Yin et al., 2009). PDX is involved in vitamin B6 biosynthesis and Arabidopsis pdx1.3 mutants strongly reduced primary root growth and increased hypersensitivity to both salt and osmotic stress (Titiz et al., 2006). Overexpression of MERI5 in Arabidopsis led to aberrant development with cell expansion alterations (Verica and Medford, 1997). O-methyltransferase, a gene encoding an enzyme involved in suberin biosynthesis, was also specifically up-regulated in RCc3:OsNAC5 roots. In Arabidopsis, transcripts of ZRP4, a gene which encodes an O-methyltransferase, were found to accumulate preferentially in the roots and localize predominantly in the endodermis region with low levels detectable in the leaves, stems and other shoot organs (Held et al., 1993). The upregulation of three O-methyltransferase genes via a root-specific promoter may have contributed to the enhanced drought tolerance of RCc3:OsNAC5 plants over both GOS2:OsNAC5 and NT plants due to their involvement in suberin biosynthesis. Lignin and suberin play major roles in impeding radial oxygen loss through lignification and/or suberization of the walls of the root peripheral layers in a process known as barrier formation. Collectively, the increased expression of such target genes in RCc3:OsNAC5 roots enlarged root tissues enhancing tolerance to drought stress at reproductive stage.

Table B below shows: Up-regulated genes in RCc3:OsNAC5 and/or GOS2:OsNAC5 plants in comparison to non-transgenic controls.

^{a}Sequence identification numbers for the full-length cDNA sequences of the corresponding genes. ^{b}Stress responsible genes to ABA (A), cold (C) drought (D) and salt (S) are based on our microarray profiling data (Accession number: GSE31874). ^{c}The mean of two independent biological replicates. Numbers in boldface indicate up-regulation by more than 3-fold (P<0.05). ^{d}P values were analyzed by one-way ANOVA. Genes discussed in the text are in boldface. These microarray data sets can be found at http://www.ncbi.nlm.nih.gov/geo/ (Gene Expression Omnibus, GEO), Accession number: GSE31856.

TABLE B

| | | RCc3: OsNAC5 | | GOS2: OsNAC5 | | ^{b}Stress |
|---|---|---|---|---|---|---|
| Gene Name | ^{a}Loc No | Mean^{b} | p-val^{c} | Mean^{b} | p-val^{c} | response |

Genes up-regulated in both RCc3: OsNAC5 and GOS2: OsNAC5 plants

| | | | | | | |
|---|---|---|---|---|---|---|
| Calcium-transporting ATPase | Os10g0418100 | 10.36 | 0.00 | 6.19 | 0.00 | C |
| Oxo-phytodienoic acid reductase | Os06g0215900 | 10.82 | 0.00 | 15.54 | 0.00 | |
| Cinnamoyl-CoA reductase | Os02g0811800 | 8.55 | 0.00 | 9.05 | 0.00 | |
| Chitinase | Os11g0701500 | 7.12 | 0.00 | 14.20 | 0.00 | |
| Cytochrome P450 | Os12g0150200 | 6.37 | 0.00 | 4.79 | 0.00 | C, D, S |
| CBS protein | Os02g0639300 | 6.04 | 0.00 | 3.70 | 0.00 | |
| Sulfotransferase | Os01g0311600 | 5.24 | 0.00 | 7.60 | 0.00 | |
| Aminotransferase | Os05g0244700 | 5.18 | 0.00 | 6.05 | 0.00 | A, D, S |
| Chitinase | Os11g0701000 | 4.97 | 0.00 | 14.04 | 0.00 | |
| Multicopper oxidase | Os01g0127000 | 4.69 | 0.00 | 4.91 | 0.00 | |
| Nicotianamine synthase | Os07g0689600 | 4.70 | 0.00 | 5.15 | 0.00 | |
| Pathogenesis-related transcriptional factor | Os07g0674800 | 4.09 | 0.00 | 12.03 | 0.00 | |
| Cinnamoyl-CoA reductase | Os02g0808800 | 4.14 | 0.00 | 11.52 | 0.00 | |
| Cinnamyl alcohol dehydrogenase | Os04g0612700 | 3.90 | 0.00 | 17.61 | 0.00 | |
| ZIM | Os03g0180900 | 4.06 | 0.00 | 3.07 | 0.00 | A, C, D, S |
| Glycoside hydrolase | Os05g0247800 | 4.07 | 0.00 | 4.04 | 0.00 | A, S |
| Glutathione-S-transferase | Os10g0530500 | 3.88 | 0.00 | 4.81 | 0.00 | |
| Iron-phytosiderophore transporter | Os02g0649900 | 3.86 | 0.00 | 5.40 | 0.00 | |
| Aminotransferase | Os01g0729600 | 3.21 | 0.00 | 15.45 | 0.00 | |
| Oxidase | Os06g0548200 | 3.61 | 0.00 | 3.82 | 0.00 | |
| Disease resistance response protein | Os07g0643800 | 3.07 | 0.00 | 3.45 | 0.00 | |
| WRKY | Os06g0649000 | 3.39 | 0.00 | 5.62 | 0.00 | D, S |
| Acyltransferase | Os03g0245700 | 3.06 | 0.00 | 3.76 | 0.00 | |
| Pyruvate kinase | Os04g0677300 | 3.01 | 0.00 | 3.66 | 0.00 | |
| Oxidative stress response protein | Os03g0830500 | 3.32 | 0.00 | 4.07 | 0.00 | D, S |

Genes up-regulated in RCc3: OsNAC5 plants

| | | | | | | |
|---|---|---|---|---|---|---|
| GLP | Os03g0694000 | 32.65 | 0.00 | 1.05 | 0.00 | A, S |
| C4-dicarboxylate transporter | Os04g0574700 | 30.10 | 0.00 | 1.11 | 0.00 | |
| O-methyltransferase | Os10g0118200 | 16.47 | 0.00 | −1.46 | 0.00 | A, S |
| Fructose-bisphosphate aldolase | Os08g0120600 | 11.27 | 0.00 | 1.01 | 0.00 | D, S |
| O-methyltransferase | Os09g0344500 | 8.43 | 0.00 | −1.09 | 0.00 | A, S |
| MtN | Os05g0426000 | 7.86 | 0.00 | 1.62 | 0.00 | |
| O-methyltransferase | Os10g0118000 | 7.09 | 0.00 | −2.23 | 0.00 | S |
| Dehydration-responsive protein | Os11g0170900 | 6.10 | 0.00 | 1.24 | 0.00 | D |
| Lipid transfer protein | Os01g0822900 | 5.06 | 0.00 | 1.61 | 0.00 | |
| Oxidase | Os03g0693900 | 4.86 | 0.00 | 1.99 | 0.00 | A, S |
| Glutamine synthetase | Os03g0712800 | 4.16 | 0.00 | 1.25 | 0.00 | |
| Lipid transfer protein | Os11g0115400 | 3.71 | 0.00 | 1.87 | 0.00 | A |
| PDX | Os07g0100200 | 3.61 | 0.00 | 1.77 | 0.00 | |
| Cytochrome P450 | Os01g0804400 | 3.61 | 0.00 | 1.10 | 0.00 | |
| MERI5 | Os04g0604300 | 3.57 | 0.00 | 1.41 | 0.00 | |
| Homeobox | Os06g0317200 | 3.33 | 0.00 | −1.97 | 0.00 | |
| Pectin acetylesterase | Os01g0319000 | 3.24 | 0.00 | −1.50 | 0.00 | |
| bZIP | Os02g0191600 | 3.20 | 0.00 | −1.73 | 0.00 | |
| Lipid transfer protein | Os12g0115000 | 3.08 | 0.00 | 1.76 | 0.00 | |

Genes up-regulated in GOS2: OsNAC5 plants

| | | | | | | |
|---|---|---|---|---|---|---|
| Glutathione S-transferase | Os09g0367700 | 1.30 | 0.00 | 10.26 | 0.00 | A, D, S |
| Serine/threonine protein kinase | Os03g0269300 | 1.51 | 0.00 | 8.70 | 0.00 | |
| WRKY | Os03g0335200 | 1.18 | 0.00 | 7.56 | 0.00 | |
| Heavy metal transport/detoxification protein | Os04g0464100 | 1.20 | 0.00 | 6.78 | 0.00 | |
| Stress response protein | Os01g0959100 | −1.09 | 0.00 | 4.76 | 0.00 | C, D, S |
| Auxin efflux carrier | Os08g0529000 | 1.19 | 0.00 | 4.52 | 0.00 | |
| Subtilase | Os02g0270200 | 1.93 | 0.00 | 4.41 | 0.00 | |
| UDP-glucuronosyl/UDP-glucosyltransferase | Os01g0638000 | 1.23 | 0.00 | 4.59 | 0.00 | A, S |
| Disease resistance protein | Os06g0279900 | −2.53 | 0.00 | 4.84 | 0.00 | |
| Nitrate reductase | Os02g0770800 | −1.26 | 0.00 | 4.85 | 0.00 | C |
| Heat shock protein | Os01g0606900 | 1.66 | 0.00 | 4.44 | 0.00 | A, D, S |
| Phosphoenolpyruvate carboxykinase | Os10g0204400 | 1.28 | 0.00 | 3.29 | 0.00 | |
| Xyloglucan endotransglycosylase | Os02g0280300 | −2.13 | 0.00 | 3.95 | 0.00 | |
| Isopenicillin N synthase | Os05g0560900 | 1.98 | 0.00 | 3.25 | 0.00 | |
| Zinc finger | Os03g0820300 | 1.61 | 0.00 | 3.44 | 0.00 | D, S |
| Serine/threonine protein kinase | Os09g0418000 | 1.60 | 0.00 | 3.07 | 0.00 | A |
| ATPase | Os03g0584400 | 1.38 | 0.00 | 3.62 | 0.00 | |
| Malic enzyme | Os05g0186300 | 1.88 | 0.00 | 3.06 | 0.00 | |

### SEQ ID NO:s for the Sequences in Table B Above

| | | |
|---|---|---|
| gene | Loc No | SEQ ID NO: |

Genes up-regulated in both RCc3: OsNAC5 and GOS2: OsNAC5 plants

| | | | |
|---|---|---|---|
| 1 | Calcium-transporting ATPase | Os10g0418100 | SEQ ID NO: 213 & 214 |
| 2 | Oxo-phytodienoic acid reductase | Os06g0215900 | SEQ ID NO: 215 & 216 |
| 3 | Cinnamoyl-CoA reductase | Os02g0811800 | SEQ ID NO: 217 & 218 |
| 4 | Chitinase | Os11g0701500 | SEQ ID NO: 219 & 220 |
| 5 | Cytochrome P450 | Os12g0150200 | SEQ ID NO: 221 & 222 |
| 6 | CBS protein | Os02g0639300 | SEQ ID NO: 223 & 224 |
| 7 | Sulfotransferase | Os01g0311600 | SEQ ID NO: 225 & 226 |
| 8 | Aminotransferase | Os05g0244700 | SEQ ID NO: 227 & 228 |
| 9 | Chitinase | Os11g0701000 | SEQ ID NO: 229 & 230 |
| 10 | Multicopper oxidase | Os01g0127000 | SEQ ID NO: 231 & 232 |
| 11 | Nicotianamine synthase | Os07g0689600 | SEQ ID NO: 233 & 234 |
| 12 | Pathogenesis-related transcriptional factor | Os07g0674800 | SEQ ID NO: 235 & 236 |
| 13 | Cinnamoyl-CoA reductase | Os02g0808800 | SEQ ID NO: 237 & 238 |
| 14 | Cinnamyl alcohol dehydrogenase | Os04g0612700 | SEQ ID NO: 239 & 240 |
| 15 | ZIM | Os03g0180900 | SEQ ID NO: 241 & 242 |
| 16 | Glycoside hydrolase | Os05g0247800 | SEQ ID NO: 243 & 244 |
| 17 | Glutathione-S-transferase | Os10g0530500 | SEQ ID NO: 245 & 246 |
| 18 | Iron-phytosiderophore transporter | Os02g0649900 | SEQ ID NO: 247 & 248 |
| 19 | Aminotransferase | Os01g0729600 | SEQ ID NO: 249 & 250 |
| 20 | Oxidase | Os06g0548200 | SEQ ID NO: 251 & 252 |
| 21 | Disease resistance response protein | Os07g0643800 | SEQ ID NO: 253 & 254 |
| 22 | WRKY | Os06g0649000 | SEQ ID NO: 255 & 256 |
| 23 | Acyltransferase | Os03g0245700 | SEQ ID NO: 257 & 258 |
| 24 | Pyruvate kinase | Os04g0677300 | SEQ ID NO: 259 & 260 |
| 25 | Oxidative stress response protein | Os03g0830500 | SEQ ID NO: 261 & 262 |

Genes up-regulated in RCc3: OsNAC5 plants

| | | | |
|---|---|---|---|
| 1 | GLP | Os03g0694000 | SEQ ID NO: 263 & 264 |
| 2 | C4-dicarboxylate transporter | Os04g0574700 | SEQ ID NO: 265 & 266 |
| 3 | O-methyltransferase | Os10g0118200 | SEQ ID NO: 267 & 268 |
| 4 | Fructose-bisphosphate aldolase | Os08g0120600 | SEQ ID NO: 269 & 270 |
| 5 | O-methyltransferase | Os09g0344500 | SEQ ID NO: 271 & 272 |
| 6 | MtN | Os05g0426000 | SEQ ID NO: 273 & 274 |
| 7 | O-methyltransferase | Os10g0118000 | SEQ ID NO: 275 & 276 |
| 8 | Dehydration-responsive protein | Os11g0170900 | SEQ ID NO: 277 & 278 |
| 9 | Lipid transfer protein | Os01g0822900 | SEQ ID NO: 279 & 280 |
| 10 | Oxidase | Os03g0693900 | SEQ ID NO: 281 & 282 |
| 11 | Glutamine synthetase | Os03g0712800 | SEQ ID NO: 283 & 284 |
| 12 | Lipid transfer protein | Os11g0115400 | SEQ ID NO: 285 & 286 |
| 13 | PDX | Os07g0100200 | SEQ ID NO: 287 & 288 |
| 14 | Cytochrome P450 | Os01g0804400 | SEQ ID NO: 289 & 290 |
| 15 | MERI5 | Os04g0604300 | SEQ ID NO: 291 & 292 |
| 16 | Homeobox | Os06g0317200 | SEQ ID NO: 293 & 294 |
| 17 | Pectin acetylesterase | Os01g0319000 | SEQ ID NO: 295 & 296 |
| 18 | bZIP | Os02g0191600 | SEQ ID NO: 297 & 298 |
| 19 | Lipid transfer protein | Os12g0115000 | SEQ ID NO: 299 & 300 |

Genes up-regulated in GOS2: OsNAC5 plants

| | | | |
|---|---|---|---|
| 1 | Glutathione S-transferase | Os09g0367700 | SEQ ID NO: 301 & 302 |
| 2 | Serine/threonine protein kinase | Os03g0269300 | SEQ ID NO: 303 & 304 |
| 3 | WRKY | Os03g0335200 | SEQ ID NO: 305 & 306 |
| 4 | Heavy metal transport/detoxification | Os04g0464100 | SEQ ID NO: 307 & 308 |
| | protein | | |
| 5 | Stress response protein | Os01g0959100 | SEQ ID NO: 309 & 310 |
| 6 | Auxin efflux carrier | Os08g0529000 | SEQ ID NO: 311 & 312 |
| 7 | Subtilase | Os02g0270200 | SEQ ID NO: 313 & 314 |
| 8 | UDP-glucuronosyl/UDP-glucosyltrans- | Os01g0638000 | SEQ ID NO: 315 & 316 |
| | Ferase | | |
| 9 | Disease resistance protein | Os06g0279900 | SEQ ID NO: 317 & 318 |
| 10 | Nitrate reductase | Os02g0770800 | SEQ ID NO: 319 & 320 |
| 11 | Heat shock protein | Os01g0606900 | SEQ ID NO: 321 & 322 |
| 12 | Phosphoenolpyruvate carboxykinase | Os10g0204400 | SEQ ID NO: 323 & 324 |
| 13 | Xyloglucan endotransglycosylase | Os02g0280300 | SEQ ID NO: 325 & 326 |
| 14 | Isopenicillin N synthase | Os05g0560900 | SEQ ID NO: 327 & 328 |
| 15 | Zinc finger | Os03g0820300 | SEQ ID NO: 329 & 330 |
| 16 | Serine/threonine protein kinase | Os09g0418000 | SEQ ID NO: 331 & 332 |
| 17 | ATPase | Os03g0584400 | SEQ ID NO: 333 & 334 |
| 18 | Malic enzyme | Os05g0186300 | SEQ ID NO: 335 & 336 |

### Example 7

### Identification of Sequences Related to SEQ ID NO: 1, SEQ ID NO: 2, SEQ ID NO: 3 and SEQ ID NO: 4

Sequences (full length cDNA, ESTs or genomic) related to SEQ ID NO: 1 and SEQ ID NO: 2 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid of SEQ ID NO: 1 was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

TABLE C
NAC1 (SEQ ID NO: 22 to SEQ ID NO: 35) and NAC5 (SEQ ID NO:
36 to SEQ ID NO: 47) nucleic acids and polypeptides:

| | Nucleic acid | Protein |
|---|---|---|
| Plant Source | SEQ ID NO: | SEQ ID NO: |
| Phyllostachys edulis | SEQ ID NO: 22 | SEQ ID NO: 23 |
| Sorghum bicolour | SEQ ID NO: 24 | SEQ ID NO: 25 |
| Zea mays | SEQ ID NO: 26 | SEQ ID NO: 27 |
| Triticum aestivum | SEQ ID NO: 28 | SEQ ID NO: 29 |
| Hordeum vulgare | SEQ ID NO: 30 | SEQ ID NO: 31 |
| Eleusine coracana | SEQ ID NO: 32 | SEQ ID NO: 33 |
| Vitis vinifera | SEQ ID NO: 34 | SEQ ID NO: 35 |
| Phyllostachys edulis | SEQ ID NO: 36 | SEQ ID NO: 37 |
| Hordeum vulgare | SEQ ID NO: 38 | SEQ ID NO: 39 |
| Sorghum bicolour | SEQ ID NO: 40 | SEQ ID NO: 41 |
| Zea mays | SEQ ID NO: 42 | SEQ ID NO: 43 |
| Vitis vinifera | SEQ ID NO: 44 | SEQ ID NO: 45 |
| Populus trichocarpa | SEQ ID NO: 46 | SEQ ID NO: 47 |

Sequences have been tentatively assembled and publicly disclosed by research institutions, such as The Institute for Genomic Research (TIGR; beginning with TA). For instance, the Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, e.g. for certain prokaryotic organisms, such as by the Joint Genome Institute. Furthermore, access to proprietary databases, has allowed the identification of novel nucleic acid and polypeptide sequences.

### Example 8

### Alignment of NCG Polypeptide Sequences

Alignment of the polypeptide sequences was performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment. See FIGS. 9 and 10.

### Example 9

### Calculation of Global Percentage Identity Between Polypeptide Sequences

Global percentages of similarity and identity between full length polypeptide sequences useful in performing the methods of the invention is determined using MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT generates similarity/identity matrices for DNA or protein sequences without needing pre-alignment of the data. The program performs a series of pair-wise alignments using the Myers and Miller global alignment algorithm, calculates similarity and identity, and then places the results in a distance matrix.

A MATGAT table based on subsequences of a specific domain is generated, which can be based on a multiple alignment of NUG polypeptides. Conserved sequences are selected for MaTGAT analysis. This approach is useful where overall sequence conservation among NUG proteins is rather low.

### Example 10

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

### Example 11

### Topology Prediction of the NCG Polypeptide Sequences

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. For the sequences predicted to contain an N-terminal presequence a potential cleavage site can also be predicted. TargetP is maintained at the server of the Technical University of Denmark.

A number of parameters are selected before analysing a sequence: such as organism group (non-plant or plant), cutoff sets (none, predefined set of cutoffs, or user-specified set of cutoffs), and the calculation of prediction of cleavage sites (yes or no).

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### Example 12

### Transformation of Other Crops

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M patent U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7 Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

Alfalfa Transformation A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2504, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Sugarbeet Transformation

Seeds of sugarbeet (Beta vulgaris L.) are sterilized in 70% ethanol for one minute followed by 20 min. shaking in 20% Hypochlorite bleach e.g. Clorox® regular bleach (commercially available from Clorox, 1221 Broadway, Oakland, Calif. 94612, USA). Seeds are rinsed with sterile water and air dried followed by plating onto germinating medium (Murashige and Skoog (MS) based medium (Murashige, T., and Skoog., 1962. Physiol. Plant, vol. 15, 473-497) including B5 vitamins (Gamborg et al.; Exp. Cell Res., vol. 50, 151-8.) supplemented with 10 g/l sucrose and 0.8% agar). Hypocotyl tissue is used essentially for the initiation of shoot cultures according to Hussey and Hepher (Hussey, G., and Hepher, A., 1978. Annals of Botany, 42, 477-9) and are maintained on MS based medium supplemented with 30 g/l sucrose plus 0.25 mg/l benzylamino purine and 0.75% agar, pH 5.8 at 23-25° C. with a 16-hour photoperiod. Agrobacterium tumefaciens strain carrying a binary plasmid harbouring a selectable marker gene, for example nptII, is used in transformation experiments. One day before transformation, a liquid LB culture including antibiotics is grown on a shaker (28° C., 150 rpm) until an optical density (O.D.) at 600 nm of ˜1 is reached. Overnight-grown bacterial cultures are centrifuged and resuspended in inoculation medium (O.D. ˜1) including Acetosyringone, pH 5.5. Shoot base tissue is cut into slices (1.0 cm×1.0 cm×2.0 mm approximately). Tissue is immersed for 30s in liquid bacterial inoculation medium. Excess liquid is removed by filter paper blotting. Co-cultivation occurred for 24-72 hours on MS based medium incl. 30 g/l sucrose followed by a non-selective period including MS based medium, 30 g/l sucrose with 1 mg/l BAP to induce shoot development and cefotaxim for eliminating the Agrobacterium. After 3-10 days explants are transferred to similar selective medium harbouring for example kanamycin or G418 (50-100 mg/l genotype dependent). Tissues are transferred to fresh medium every 2-3 weeks to maintain selection pressure. The very rapid initiation of shoots (after 3-4 days) indicates regeneration of existing meristems rather than organogenesis of newly developed transgenic meristems. Small shoots are transferred after several rounds of subculture to root induction medium containing 5 mg/l NAA and kanamycin or G418. Additional steps are taken to reduce the potential of generating transformed plants that are chimeric (partially transgenic). Tissue samples from regenerated shoots are used for DNA analysis. Other transformation methods for sugarbeet are known in the art, for example those by Linsey & Gallois (Linsey, K., and Gallois, P., 1990. Journal of Experimental Botany; vol. 41, No. 226; 529-36) or the methods published in the international application published as WO9623891A.

### Sugarcane Transformation

Spindles are isolated from 6-month-old field grown sugarcane plants (Arencibia et al., 1998. Transgenic Research, vol. 7, 213-22; Enriquez-Obregon et al., 1998. Planta, vol. 206, 20-27). Material is sterilized by immersion in a 20% Hypochlorite bleach e.g. Clorox® regular bleach (commercially available from Clorox, 1221 Broadway, Oakland, Calif. 94612, USA) for 20 minutes. Transverse sections around 0.5 cm are placed on the medium in the top-up direction. Plant material is cultivated for 4 weeks on MS (Murashige, T., and Skoog., 1962. Physiol. Plant, vol. 15, 473-497) based medium incl. B5 vitamins (Gamborg, O., et al., 1968. Exp. Cell Res., vol. 50, 151-8) supplemented with 20 g/l sucrose, 500 mg/l casein hydrolysate, 0.8% agar and 5 mg/l 2,4-D at 23° C. in the dark. Cultures are transferred after 4 weeks onto identical fresh medium. Agrobacterium tumefaciens strain carrying a binary plasmid harbouring a selectable marker gene, for example hpt, is used in transformation experiments. One day before transformation, a liquid LB culture including antibiotics is grown on a shaker (28° C., 150 rpm) until an optical density (O.D.) at 600 nm of ˜0.6 is reached. Overnight-grown bacterial cultures are centrifuged and resuspended in MS based inoculation medium (O.D. ˜0.4) including acetosyringone, pH 5.5. Sugarcane embryogenic callus pieces (2-4 mm) are isolated based on morphological characteristics as compact structure and yellow colour and dried for 20 min. in the flow hood followed by immersion in a liquid bacterial inoculation medium for 10-20 minutes. Excess liquid is removed by filter paper blotting. Co-cultivation occurred for 3-5 days in the dark on filter paper which is placed on top of MS based medium incl. B5 vitamins containing 1 mg/l 2,4-D. After co-cultivation calli are washed with sterile water followed by a non-selective cultivation period on similar medium containing 500 mg/l cefotaxime for eliminating remaining Agrobacterium cells. After 3-10 days explants are transferred to MS based selective medium incl. B5 vitamins containing 1 mg/l 2,4-D for another 3 weeks harbouring 25 mg/l of hygromycin (genotype dependent). All treatments are made at 23° C. under dark conditions. Resistant calli are further cultivated on medium lacking 2,4-D including 1 mg/l BA and 25 mg/l hygromycin under 16 h light photoperiod resulting in the development of shoot structures. Shoots are isolated and cultivated on selective rooting medium (MS based including, 20 g/l sucrose, 20 mg/l hygromycin and 500 mg/l cefotaxime). Tissue samples from regenerated shoots are used for DNA analysis. Other transformation methods for sugarcane are known in the art, for example from the in-ternational application published as WO2010/151634A and the granted European patent EP1831378.

### REFERENCES

Clark, L. J., Price, A. H., Steel, K. A. and Whaley, W. R. (2008) Evidence from near-isogenic lines that root penetration increases with root diameter and bending stiffness in rice. Funct Plant Biol 35: 1163-1171.
Cai, X., Chen, T., Zhou, Q. Y., Xu, L., Qu, L. Q., Hua, X. J. and Lin, J. X. (2011) Development of casparian strip in rice cultivars. Plant Sig & Behavior 6: 59-65.
de Pater, B. S., van der Mark, F., Rueb, S., Katagiri, F., Chua, N. H., Schilperoort, R. A. and Hensgens, L. S. (1992) The promoter of the rice gene GOS2 is active in various different monocot tissues and bind rice nuclear factor ASF-1. Plant J 2: 837-844.
Drew, M. C., He, C. J. and Morgan, P. W. (2000) Programmed cell death and aerenchyma formation in roots. Trends Plant Sci 5:123-127.
Ernst, E. A., Olsen, A. N., Skriver, K., Larsen, S. and Leggio, L. L. (2004) Structure of the conserved domain of ANAC, a member of the NAC family of transcription factors. EMBO Rep 5:297-303.
Fan, L., Linker, R., Gepstein, S., Tanimoto, E., Yamamoto, R. and Neumann, P. M. (2006) Progressive inhibition by water deficit of cell wall extensibility and growth along the elongation zone of maize roots is related to increased lignin metabolism and progressive stelar accumulation of wall phenolics. Plant Physiol 140: 603-612.
Fitter, A. H. (1991) The ecological significance of root system architecture: an economic approach. In: Plant Root Growth: An Ecological Perspective. Blackwell Scientific Publishers, London.
Goujon, T., Ferret, V., Mila, I., Pollet, B., Ruel, K., Burlat, V., Joseleau, J. P., Barrière, Y., Lapierre, C., Jounanin, L. (2003) Down-regulation of the AtCCR1 gene in Arabidopsis thaliana: effects on phenotype, lignins and cell wall degradability. Plana 217: 218-228
Hegedus, D., Yu, M., Baldwin, D., Gruber, M., Sharpe, A., Parkin, I., Whitwill, S. and Lydiate, D. (2003) Molecular characterization of Brassica napus NAC domain transcriptional activators induced in response to biotic and abiotic stress. Plant Mol Biol 53: 383-397.
Held, B. M., Wang, H., John, I., Wurtele, E. S. and Colbert, J. T. (1993) An mRNA putatively coding for an o-methyltransferase accumulates preferentially in maize roots and is located predominantly in the region of the endodermis. Plant Physiol 102: 1001-1008.
Ingram, K. T., Bueno, F. D., Namuco, O. S., Yambao, E. B. and Beyrouty, C. A. (1994) Rice root traits for drought resistance and their genetic variation. In: Kirk G J D (Ed.), Rice Roots: Nutrient and Water Use. International Rice Research Institute, Manila, Philippines.
Jang, I. C., Choi, W. B., Lee, K. H., Song, S. K., Nahm, B. K. and Kim, J. K. (2002) High-level and ubiquitous expression of the rice cytochrome c gene OsCc1 and its promoter activity in transgenic plants provides a useful promoter for transgenesis of monocots. Plant Physiol 129: 1473-1481.
Jang, I. C., Nahm, B. H. and Kim, J. K. (1999) Subcellular targeting of green fluorescent protein to plastids in transgenic rice plants provides a high-level expression system. Mol Breed 5: 453-461.
Jeong, J. S., Park, Y. T., Jung, H., Park, S. H. and Kim, J. K. (2009) Rice NAC proteins act as homodimers and heterodimers. Plant Biotechonol Rep 3: 127-134.
Jeong, J. S., Kim, Y. S., Baek, K. H., Jung, H., Ha, S. H., Choi, Y. D., Kim, M., Reuzeau, C. and Kim, J. K. (2010) Root-specific expression of OsNAC10 improves drought tolerance and grain yield in rice under field drought conditions. Plant Physiol 153: 185-197.
Jones, L. E., Ennos, A. R. and Turner, S. R. (2001) Cloning and characterization of irregular xylem4 (irx4); a severly lignin-dependent mutant of Arabidopsis. Plant J 26: 205-216.
Knight, H. (2000) Calcium signaling during abiotic stress in plants. Int Rev Cytol 195:269-325
Ma, F. and Peterson, C. A. (2003) Current insights into the development, structure and chemistry of the endodermis and exodermis of roots. Can J Bot 81: 404-421.
Nguyen, H. T., Babu, R. C. and Blum, A. (1997) Breeding for drought resistance in rice: Physiology and molecular considerations. Crop Sci 37: 1426-1434.
Oh, S. J., Song, S. I., Kim, Y. S., Jang, H. J., Kim, S. Y., Kim, M. J., Kim, Y. K., Nam, B. H. and Kim, J. K. (2005) Arabidopsis CBF3/DREB1A and ABF3 in Transgenic Rice Increased Tolerance to Abiotic Stress without Stunting Growth. Plant Physiol 138: 341-351.
Oh, S. J., Kim, Y. S., Kwon, C. W., Park, H. K., Jeong, J. S. and Kim, J. K. (2009) Overexpression of the transcription factor AP37 in rice improves grain yield under drought conditions. Plant Physiol 150: 1368-1379.
Redillas M C F, Strasser R J, Jeong J S, Kim Y S, Kim J K (2011a) The use of JIP test to evaluate drought-tolerance of transgenic rice overexpressing OsNAC10. Plant Biotechnol Rep 5: 169-175.
Redillas M C F, Jeong J S, Strasser R J, Kim Y S, Kim J K (2011b) JIP analysis on rice (Oryza sativa cv Nipponbare) grown under limited nitrogen conditions. J Kor Soc Appl Biol Chem, doi: 10.3839.
Sattler, S. E., Saathoff, A. J., Haas, E. J., Palmer, N. A., Funnell-Harris, D. L., Sarath, G. and Pedersen, J. F. (2009) A nonsense mutation in a cinnamyl alcohol dehydrogenase gene is responsible for the Sorghum brown midrib6 phenotype. Plant Physiol 150: 584-595.
Takasaki, H., Maruyama, K., Kidokoro, S., I to, Y., Fujita, Y., Shinozaki, K., Yamaguchi-Shinozaki K. and Nakashima, K. (2010) The abiotic stress-responsive NAC-type transcription factor OsNAC5 regulates stress-inducible genes and stress tolerance in rice. Mol Genet Genomics 284: 173-183.
Tamasloukht, B., Lam, M. S. J. W. Q., Martinez, Y., Tozo, K., Barbier, O., Jourda, C., Jauneau, A., Borderies, G., Balzergue, S., Renou, J. P., Huguet, S., Martinant, J. P., Tatout, C., Lapierre, C., Barrière, Y., Goffner, D. and Pichon, M. (2011) Characterization of cinnamoyl-CoA reductase 1 (CCR1) mutant in maize: effect of lignification, fibre development, and global gene expression. J Exp Bot 62: 3837-3848.
Tan, B. C., Schwartz, S. H., Zeevart, J. A. D. and McCarty, D. R. (1997) Genetic control of abscisic acid biosynthesis in maize. Proc Natl Acad Sci USA 94: 12235-12240.
Titiz O, Tambasco-Studart M, Warzych E, Apel K, Amrhein N, Laloi C, Fitzpatrick T B (2006) PDX1 is essential for vitamin B6 biosynthesis, development and stress tolerance in Arabidopsis. Plant J 48: 933-946.
Verica J A and Medford J I (1997) Modified MERI5 expression alters cell expansion in transgenic Arabidopsis plants. Plant Science 125: 301-210
Xie, Q., Frugis, G., Colgan, D. and Chua, N. H. (2000) Arabidopsis NAC1 transduces auxin signal downstream of TIR1 to promote lateral root development. Genes Dev 14: 3024-3036.
Xu, Y., Buchholz, W. G., DeRose, R. T. and Hall, T. C. (1995) Characterization of a rice gene family encoding root-specific proteins. Plant Mol Biol 27: 237-248.
Yambao, E. B., Ingram, K. T. and Real, J. G. (1992) Root xylem influence on the water relations and drought resistance in rice. J Exp Bot 43: 925-932.
Yin K, Han X, Xu Z, Xue H (2009) Arabidopsis GLP4 is localized to the Golgi and binds auxin in vitro. Acta Biochim Biophys Sin 41: 478-487.
Zheng, X. N., Chen. B., Lu, G. J. and Han, B. (2009) Overexpression of a NAC transcription factor enhances rice drought and salt tolerance. Biochem Biophys Res Commun 379: 985-989.
Zhu J, Brown K M, Lynch J P (2010) Root cortical aerenchyma improves the drought tolerance of maize (Zea mays L.). Plant Cell Environ 33: 740-749.

1. A method for enhancing yield-related traits and/or modifying root architecture in plants grown under abiotic stress conditions, comprising introducing and expressing in a plant a nucleic acid encoding a NAC5 polypeptide represented by SEQ ID NO 4 or a NAC1 polypeptide represented by SEQ ID NO: 2, or a homologue thereof having at least 50% sequence identity to SEQ ID NO: 4 or SEQ ID NO: 2.

2. The method according to claim 1, wherein said nucleic acid is operably linked to a tissue-specific promoter, a root-specific promoter, an RCc3 promoter, or RCc3 promoter from rice.

3. The method according to claim 1, wherein said nucleic acid is operably linked to a constitutive promoter, a GOS2 promoter, or an GOS2 promoter from rice.

4. The method according to claim 1, wherein said enhanced yield-related traits comprise increased seed or grain yield.

5. The method according to claim 1, to wherein said modified root architecture comprises or is due to an increase or change in any one or more of the following: an increase in root biomass in the form of fresh weight or dry weight, increased number of roots, increased root diameter, enlarged roots, enlarged stele, enlarged aerenchyma, increased aerenchyma formation, enlarged cortex, enlarged cortical cells, enlarged xylem, modified branching, improved penetration ability, enlarged epidermis, increase in the ratio of roots to shoots.

6. The method according to claim 1, wherein said enhanced yield-related traits are obtained under conditions of drought stress or salt stress.

7. The method according to claim 1, wherein said NAC5 or NAC1 polypeptide comprises one or more of the motifs represented by SEQ ID NO: 5 to SEQ ID NO: 15.

8. The method according to claim 1, wherein said nucleic acid encoding a NAC5 or NAC1 is of plant origin, from a monocotyledonous plant, from a plant of the family Poaceae, from a plant of the genus Oryza, or from an Oryza sativa plant.

9. The method according to claim 1, wherein said nucleic acid encoding a NAC5 or NAC1 encodes any one of the polypeptides listed in Table C or is a portion of such a nucleic acid, or a nucleic acid capable of hybridising with such a nucleic acid.

10. The method according to claim 1, wherein said nucleic acid sequence encodes an orthologue, or paralogue of any of the polypeptides given in Table C.

11. The method according to claim 1, wherein said nucleic acid encodes the NAC5 polypeptide represented by SEQ ID NO: 4 or wherein said nucleic acid encodes the NAC1 polypeptide represented by SEQ ID NO: 2.

12. A plant or part thereof, or plant cell, obtainable by the method according to claim 1, wherein said plant, plant part or plant cell comprises a recombinant nucleic acid encoding a NAC5 polypeptide or a NAC1 polypeptide as given in Table C or a homologue, paralogue or orthologue thereof.

13. A construct comprising:
(i) a nucleic acid sequence encoding a NAC5 polypeptide or a NAC1 polypeptide as given in Table C or a homologue, paralogue or orthologue thereof;
(ii) one or more control sequences capable of driving expression of the nucleic acid sequence of (i) comprising at least a tissue-specific promoter; and optionally
(iii) a transcription termination sequence.

14. The construct according to claim 13, wherein said nucleic acid sequence is operably to a constitutive promoter of plant origin, a medium strength constitutive promoter of plant origin, a GOS2 promoter, or a GOS2 promoter from rice.

15. The construct according to claim 13, wherein said tissue-specific promoter is a root-specific promoter or an RCc3 promoter.

16. A method for making plants having enhanced yield-related traits, increased seed yield, increased biomass, and/or modified root architecture relative to control plants, comprising introducing the construction according to claim 13.

17. A plant, plant part or plant cell transformed with the construct according to claim 13.

18. A method for the production of a transgenic plant having enhanced yield-related traits increased seed yield, and/or increased biomass relative to control plants, comprising:
(i) introducing and expressing in a plant cell or plant a nucleic acid encoding a NAC1 or NAC5 polypeptide as given in Table C or a homologue, paralogue or orthologue thereof; and
(ii) cultivating said plant cell or plant from step (i) under abiotic stress conditions, wherein said plants have increased seed yield and modified root architecture.

19. The method according to claim 18, wherein said nucleic acid is operably linked to a tissue-specific promoter, a root-specific promoter, an RCc3 promoter, or an RCc3 promoter from rice.

20. The method according to claim 18, wherein said nucleic acid is operably linked to a constitutive promoter, a GOS2 promoter, or tan GOS2 promoter from rice.

21. A transgenic plant having enhanced yield related traits relative to control plants resulting from modulated expression of a nucleic acid encoding a NAC1 car NAC5 polypeptide as given in Table C or a homologue, paralogue or orthologue thereof.

22. The transgenic plant according to claim 21, or a transgenic plant cell derived therefrom, wherein said plant is a crop plant, a monocotyledonous plant or a cereal, or wherein said plant is beet, sugarbeet, alfalfa, sugarcane, maize, wheat, barley, millet, rye, triticale, sorghum, emmer, spelt, einkorn, teff, milo or oats.

23. Harvestable parts of the plant according to claim 22, wherein said harvestable parts are root biomass and/or seeds.

24. Products derived from the plant according to claim 22 and/or from harvestable parts of said plant.

25. (canceled)

26. A method for manufacturing a product, comprising the steps of growing the plant according to claim 12, and producing a product from or by said plant, or parts thereof, including seeds.
